(19) United States
(12) Patent Application Publication
(10) Pub. No.: US 2015/0079069 A1
(43) Pub. Date: Mar. 19, 2015

(54) METHODS FOR MODIFICATION OF TISSUES

(71) Applicant: YO PROTEINS AB, Rönninge (SE)

(72) Inventor: Aleksei Rozkov, Ronninge (SE)

(73) Assignee: ELASTOMICS AB, Ronninge (SE)

(21) Appl. No.: 14/397,378

(22) PCT Filed: Apr. 26, 2013
(86) PCT No.: PCT/SE2013/050472

§ 371 Date: Oct. 27, 2014

Related U.S. Application Data
(60) Provisional application No. 61/640,008, filed on Apr. 30, 2012.

(51) Int. Cl. C12N 9/64; A61K 38/48

(52) U.S. Cl. CPC C12N 9/6489; A61K 38/4886; C12Y 304/24035
USPC 424/94.67; 435/1.1; 435/212

(57) ABSTRACT

Methods for modification of mechanical properties or a shape of a mammalian tissue, excluding scar, blood clot, fat; including a step of contacting the tissue with an isolated eukaryotic endopeptidase in an amount sufficient to cause a modification of mechanical properties of the tissue or its shape, but insufficient to cause degradation of the tissue's into incohesive parts, and to the use of eukaryotic endopeptidases for use in methods for treatment of the mammalian body.

### FIELD OF THE INVENTION

The present invention relates to the field of methods for enzymatic modification of tissues, and especially to methods using eukaryotic endopeptidases for modification of mechanical properties or the shape of mammalian tissue.

### BACKGROUND

Mechanical properties and shape of mammalian tissues are defined by extracellular matrix.

Mechanical properties of tissues tend to change with age or due to disease. Increased organ stiffness and fragility is cause to multiple pathological conditions, such as: liver fibrosis (Wells 2005; Georges, Hui et al. 2007), liver cirrhosis (Dechene, Sowa et al. 2010), adhesive capsulitis, Dupuytren's contracture (Hurst, Badalamente et al. 2009), phimosis, aged eye lens stiffness (Hansen, Stitzel et al. 2003; Stitzel, Hansen et al. 2005).

Stiffness of healthy organs sets limitations on (1) ability to reshape tissues, desired for plastic and reconstructive surgery, (2) ability of transplant tissue to adjust to shape of host tissue without gaps, (3) ability to increase amount of transplanted tissue by means of stretching, (4) ability for scarless healing of lesions and wounds, (5) ability to heal ruptured tissues.

Stiffness of organs sets limitations on ability of organs to expand (grow). Notably, cell cultures, comprising the tissues, have high capacity for proliferation in vitro and in vivo, however, whole tissues and organ have far more limited ability to expand due to extracellular matrix stiffness.

Stiffness of extracellular matrix is known to define cell behavior, such as: adhesion, migration, proliferation and even differentiation patterns. Modification of tissue stiffness in situ could be the key to regulate cell behavior, including differentiation, within natural environment of the cell (Discher, Janmey et al. 2005; Engler, Sen et al. 2006; Engler, Sweeney et al. 2007).

Proteolytical enzymes have been used for decades in medicine, biotechnology and industry in order to degrade extracellular matrix and thus affect properties of tissues. Collagenolytic enzymes of bacterial source (Clostridium histolyticum) and crab digestive collagenases are used in medicine for decades. Those collagenases are highly efficient for tissues dissociation, effects being used for necrotic tissues debridement (Klasen 2000), scar removal, derivation of primary cell suspensions from tissues, islet derivation from pancreases (Li, Yuan et al. 2009).

However, use of bacterial collagenolytic enzymes envolves a risk of ruptures for treated and adjacent tissues. Collagenase from Clostridium histolyticum, when applied to Dupuytren's cords in vitro allowed 93% decrease in tensile modulus and 3,600 units dosage; however, only 300 units dose of collagenase was sufficient for cords rupture within physiological cords limits (Starkweather, Lattuga et al. 1996). Clinical trials of collagenase from Clostridium histolyticum demonstrated increase of limb mobility range after collagenase treatment, however, accompanied by adverse effects caused by integrity failure in adjacent tissues (such as: tendon rupture, blistering, pain, numbness, etc) (Hurst, Badalamente et al. 2009).

There are a number patents and scientific articles covering the use of bacterial collagenase or crab/shrimp digestive collagenase and proteolytic enzymes in surgery, regenerative medicine, cosmetology, food industry, etc.

The majority of work on MMP's and other tissue-remodelling enzymes is related to their role in cancer and mostly cover their molecular structure, search for inhibititors or use as diagnostic markers.

However, the use of MMP's and other tissue-remodelling enzymes for use in the applications for which bacterial and digestive collagenases are known has not received much attention.

There are multiple patents where a certain effect, achieved with bacterial collagenase was speculatively extended into all types of collagenolytic enzymes. This includes U.S. Pat. No. 6,365,405, U.S. Pat. No. 8,323,642, US 20080145357. The experimental work disclosed in these documents do however not include mammalian tissue-remodeling enzymes.

Work by Hirayasu (Hirayasu, Yoshikawa et al. 2008) shows mammalian extracellular matrix-degrading enzyme Granzyme B use to detach cells in vitro.

Use of enzymes to degrade fibrin clot, scar, fat deposits, which are temporary tissues, is also known in the art.

U.S. Pat. No. 5,922,322 and US patent application 20110091436 disclose that fibrin clot can be dissolved by MMP-3 and by MMP-10, respectively. Fibrin is a short-term temporary tissue, it is meant by nature to be dissolved after short while. Use of MMP's allows to increase the rate of dissolving the fibrin clot, therefore, disruption and desintegration effects are highly desirable.

EP0637450 claims that protelytical enzymes, including those of MMP family, can be used “to provide partial degradation of either scar tissue or matrix component”, being part of sofnening, expanding composition. Human stromelysin and collagenase, purified from human gingival fibroblasts, together with other components of softening composition, were injected into scar in order to make it more loose and soft. Scar is a temporary tissue intended to interconnect gaps between ruptured permanent tissue, however, the rate of natural dissolving of scars is too low and enzymes are required to enhance the process.

US patent application 20100247512 suggests that enhancing MT1-MMP secretion by host organism (patient's) own cells may enable fat deposition and inhibiting of MT1-MMP inhibits process of fat deposition.

WO 2009111083 describes that cathepsin L, when injected subcutaneously, partially dissolves extracellular matrix of subcutaneous fat deposits (fibrous septae).

U.S. Pat. No. 7,935,337 teaches that MMPs (namely, MMP-3 and MMP-7), when applied to herniated intervertebrate cartilage discs can partially degrade it, both in vivo and in vitro. Authors discovered that (1) “using MMP-3, MMP-7 or both in combination significantly reduced the wet weight compared to the control” and (2) “resorption of herniated disc” that was treated by those MMPs was evaluated by histochemistry staining of MMP-treated tissues with Safranin O. Authors evaluate success of the method in terms of tissue dissociation (resorbtion, degradation) efficiency. The purpose of the method described by authors is reverse to effect of tissue maintenance and functional integrity that is the subject of the present invention.

US patent application 20110044960 describes MMP-3 being delivered into tooth. Authors demonstrate proliferative, migrative, anti-apoptotic effect of MMP-3 on vascular endothelial cells in vitro and induction of odontoblasts by MMP-3 in vitro. Authors observed same benefectory effects when they applied MMP-3 to tooth in vivo, thus demonstrating stimulatory effect of MMP-3 on vascular endothelial and odonthoblast cells. However, authors did not demonstrate effects of changing mechanical or shape properties of actually tooth tissue (such as to correct positioning of tooth or to increase it's plasticity).

Prior art for using Tissue-Remodeling Enzymes on mammalian tissues are reviewed in Table 4.

### SUMMARY

In one aspect, the invention relates to a method for modification of mechanical properties or a shape of a mammalian tissue, excluding scar, blood clot, fat; comprising a step of contacting said tissue with an isolated eukaryotic endopeptidase in an amount sufficient to cause a modification of mechanical properties of said tissue or its shape, but insufficient to cause degradation of said tissue's into incohesive parts.

In a second aspect, the invention relates to eukaryotic endopeptidase for use in a method for treatment of a mammalian body.

The methods for treatment according to this aspect preferably include methods according to the first aspect as one or more parts in a complete method for treatment.

In a further aspect, the invention relates to methods for treatment of a subject using eukaryotic endopeptidases and the methods according to the first aspect.

Currently preferred embodiments are set out in the dependent claims.

### FIGURES

FIG. 1: Functional integrity of muscle and tendon. Loss of functional integrity may be result in pathologies, such as: pain, numbness, paralysis, muscle rupture, congenital muscular dystrophy or tendon rupture.

FIG. 2: Functional integrity of skin. Loss of functional integrity may result in pathologies, such as: wound, lesion, rupture, ulcer, epidermolysis bullosa, bullous pemphigoid, blistering, alopecia.

FIG. 3. Plastic materials are less prone to rupture and erosion, compared to stiff ones materials. Mechanisms of plasticity, brittleness (fragility), rupture and erosion are reviewed and illustrated in (Picu R. C. (2011), Hong W. et. al. (2008), Wang X. et. al. (2012)).

FIG. 4: Adhesive capsulitis: stress-strain characteristics. Reduced stress-strain ratio (as after TREZ-treatment) allow to increase motility range. Implications for adhesive capsulitis (frozen shoulder).

FIG. 5: Phimosis: stress-strain characteristics. Reduced stress-strain ratio (as after TREZ-treatment) allow to increase painless deformation range. Implications for phimosis.

FIG. 6: Dental braces method: stress-strain characteristics. Reduced stress-strain ratio (as after TREZ-treatment) allow to increase painless deformation range. Implications for teeth positioning correction using dental braces (continued: FIG. 9B).

FIG. 7: Cohesive tendon rearrangement, scheme. Cohesive rearrangement of tendon by MMP-9, compared to non-cohesive split by aggressive collagenases. Cohesive split allows to improve permeability.

FIG. 8. MMP-9 allows cohesive tendon rearrangement (I) (microphotographs). Large limbal tendon, treated by MMP-9, 1 mg/ml improves plasticity and permeability and reduces stiffness, however, maintaining functional integrity.

Top panel: limbal tendon, treated by MMP-9, before and after treatment. The treated tendon is split into multiple interconnected fibrils thus reducing tendon stiffness, enhancing plasticity and permeability. Importantly, the individual fibrils remain interconnected as meshwork, therefore, the tendon remains a one whole entity.

Middle panel: limbal tendon, treated by buffer, retains stiffness, inability to plastic modification and of low permeability due to dense packing.

Bottom panel: limbal tendon, treated by collagenase from Clostridium histolyticum, 1 mg/ml, before and after treatment. The treated tendon is split into multiple floating microscopic particles, therefore, functional integrity is lost.

FIG. 9. MMP-9 allows cohesive tendon rearrangement (II) (microphotographs). Large limbal tendon, treated by MMP-9 and by collagenase from Clostridium histolyticum: differences in plastic deformation vs integrity.

Top panel: tendon, treated by MMP-9, 0.1 mg/ml, is split into multiple interconnected fibrils thus reducing tendon stiffness, enhancing plasticity and permeability. Importantly, the individual fibrils remain interconnected as meshwork, therefore, the tendon remains a one whole entity.

Middle panel: limbal tendon, treated by buffer, retains stiffness, inability to plastic modification and of low permeability due to dense packing.

Bottom panel: limbal tendon, treated by collagenase from Clostridium histolyticum, 0.1 mg/ml, before and after treatment. The treated tendon is partially degraded into multiple floating microscopic particles. The remaining, undigested part of tendon, remains thin and prone to rupture; however, plasticity is not improved (remaining fibrils within the tendon are still tightly packed).

FIG. 10: Cohesive pancreas rearrangement, scheme. Cohesive split of pancreas by MMP-9, compared to non-cohesive split by aggressive collagenases. Cohesive split allows to improve permeability.

FIG. 11. MMP-9 allows cohesive pancreatic tissue rearrangement (microphotographs). MMP-9 treatment splits pancreas tissue from solid piece into thin interconnected lobules with intact duct system.

Left panel: pancreas tissue specimen, treated by MMP-9.

Right panel: pancreas specimen of same size, treated by buffer.

Both samples were treated for 24 hours at 37 C and were examined under phase contrast microscope. MMP-9 treatment splits pancreatic tissue into thin interconnected lobules (whole system of exocrine ducts, lumens and local cell niches remains intactact and tissue remains one whole entity). Reduction of tissue specimen thickness improves permeability of vitro cultures.

FIG. 12: Plasticity and adhesivity: effect in transplantation and implantation. Improved plasticity and adhesivity of TREZ-treated transplanted tissues allows to improve process of implantation.

FIG. 13: Lip tissue, treated by MMP-9 vs buffer (quantitive stress-strain characteristics).

Panels A, B (top): Stress-strain curve (X: stress applied to the tissue sample, in relative units; Y: resulting strain of the tissue sample, in %)

Panels C, D (middle): Relative area of tissue sample resulting from stress applied (X: stress, in relative units; Y: relative area of the tissue sample)

Panels E, F (bottom). Thickness of the tissue sample resulting from stress applied (X: stress, in relative units, Y: relative thickness of the tissue sample)

Left panels demonstrate stress-strain characteristics of tissue, resulting from permanent stress applied. Right panels describe plasticity: residual strain and deformation characteristics after temporary stress (pressure) applied and removed.

FIG. 14. Increased plasticity of lip connective tissue after MMP-9 treatment (microphotographs). MMP-9 treatment of subcutaneous connective tissues of lip renders them highly plastic. Top panel: MMP-9, 0.1 mg/ml, treated tissues of lip. Bottom panel: buffer-treated tissues of lip. Both specimen were treated for 4 days at 37 C and subject by temporary stress assay.

FIG. 15. MMP-9 treatment of subcutaneous connective tissues of lip renders them gel-like and adhesive.

Top panel: MMP-9, 0.1 mg/ml, treated tissues of lip. Bottom panel: buffer-treated tissues of lip. Both specimen were treated for 4 days at 37 C and were subject to qualitative tests. MMP-9 treated sample is more loose, easily adjusts it's shape, is highly adhesive and behaves like a viscous, rather than elastic, body in creep assay.

FIG. 16: Lip tissues, treated by Cathepsin B vs buffer (quantitive stress-strain characteristics). See FIG. 13 for detailed description.

FIG. 17: Dermis, treated by MMP-9 (quantitive stress-strain characteristics). See FIG. 13 for detailed description.

FIG. 18: Cartilage, treated by MMP-9 (quantitive stress-strain characteristics). See FIG. 13 for detailed description.

FIG. 19: Liver, treated by MMP-9 (quantitive stress-strain characteristics). See FIG. 13 for detailed description.

FIG. 20. MMP-9 treatment of liver tissue in vitro makes it (1) less stiff, (2) more plastic and (3) more cohesive, compared by untreated liver tissue.

MMP-9 (top) and buffer-treated (bottom) tissue samples before any stress (load) applied (left panels) vs after temporary stress of 15 relative units is applied. Noticably, MMP-9-treated liver tissue is more cohesive and less prone to ruptures under stress.

FIG. 21: Uterus tissues, treated by MMP-9 (quantitive stress-strain characteristics). See FIG. 13 for detailed description.

FIG. 22: Ovary, treated by MMP-9 (quantitive stress-strain characteristics). See FIG. 13 for detailed description.

FIG. 23. Islet: fragility assay. MMP-9 treated pancreatic insulin-producing islets of Langerhans are (1) better spread under load, and (2) less prone to rupture.

Left panel: buffer treated islet: rupture under load. Right panel: MMP-9 treated islet is spread under load from spherical formation into thin layer, the cells within the islet remain cohesive, the isled does not undergo rupture under load.

FIG. 24: Eye lens, fragility assay. MMP-9 renders eye lens more resistant to rupture under stress applied.

Left panel: eye lens, treated by MMP-9, 0.1 mg/ml. Right panel: eye lens of same animal, treated by buffer. Both samples were treated for 4 days at 37 C and were subject to temporary pressure of same intensity and duration. MMP-9 treated sample reacted to pressure by elastic deformation, while buffer-treated sample reacted in ruptures.

### DETAILED DESCRIPTION OF THE INVENTION

Although current methods using proteolytic enzymes in medicine are highly efficient in applications that aim to dissociate or otherwise destruct tissues (such as scars, necrotic tissues of burn wounds, contracted cords), there is an unmet need related to modification of mechanical or geometrical properties of tissues while maintaining functional integrity of treated and adjacent tissues.

According to the invention, Tissue-Remodeling EnZymes (TREZ), which are proteolytic enzymes of eukaryotic organisms that are produced by host organisms in order to perform remodeling of hosts own permanent tissues while maintaining their function are used to modify mechanical and geometrical properties of permanent mammalian tissues while integrity and cohesion of treated tissues remain on level sufficient to preserve tissue function. TREZ include MMPs and other mammalian collagenolytic enzymes.

The present description demonstrates effects of: reduced stiffness, reduced stress-strain ratio, increased plasticity, increased adhesiveness, reduced fragility on a wide range of mammalian tissues, after treatment by TREZ and having direct implementation for stiffness-associated diseases (fibrosis, cirrhosis, adhesive capsulitis, Dupuytren's contracture), transplantation, plastic and reconstructive surgery, reproductive medicine, dentistry, ophthalmology and biotechnology.

We have discovered that tissue-remodeling enzymes of mammalian organism (including MMPs and cathepsins) have ability to modify mechanical properties and shape of permanent tissues while maintaining their functional integrity.

The following changes in TREZ-treated tissues were observed:

(I) tissues became more elastic: (1) reduced stress-strain ratio (reduced stiffness), (2) increased plasticity (ability to adapt shape; residual deformation after temporary stress), (3) reduced fragility (brittleness). Less pressure was required to achieve certain deformation after TREZ-treatment. Fixed pressure resulted in higher extent of tissue deformation after TREZ-treatment.

(II) tissues became more plastic (gel-like): (1) tissues, that used to behave like elastic solid materials prior to treatment, after TREZ treatment behaved like gels, (2) increased viscoelasticity, (3) reduced risk for ruptures, (4) reduced risk for erosion, (5) increased adhesivity to surfaces and other tissues, (6) increased ability to modify shape, (7) increased cohesion within treated tissue.

(III) modification of shape: (1) increased ability to stretch (increase area), (2) increased ability to reduce thickness, (3) increased ability to change shape, (4) increased ability to mold into required shape, (5) improved permeability within treated tissue sample.

(IV) integrity maintenance for adjacent tissues: (1) nerve integrity, (2) tendon integrity, (3) muscle integrity, etc.

The effects were observed for various tissue types of mammalian tissues: skin, tendon, cartilage, liver, pancreas, ovaries, pancreatic islets of Langerhans, muscle, eye lens, connective tissues of penis, uterus, lip and other tissues. All the tissues maintained functional integrity while treated by such compositions of tissue-remodeling enzymes that enabled modification of tissue stiffness, plasticity, adhesivity and other mechanic properties. It is important that enzyme formulation, sufficient for plastic deformation of one tissue, would not rupture or damage integrity of adjacent tissues, such as nerve.

Further we shall describe implementations of tissue-remodeling enzyme caused effect on permanent tissues in range of medical, biotechnological and industrial applications, such as:
1) need to modify tissue shape in plastic surgery, dentistry and cosmetics
2) need for increased plasticity and adhesivity of tissues in transplantation
3) need to reduce tissue stiffness in fibrosis and cirrhosis
4) need to reduce stiffness in order to increase healthy organ motility range in such conditions as adhesive capsulitis (frozen shoulder), phimosis, aged eye lens stiffening, Dupuytren's contracture, etc.

### Extracellular Matrix and Collagen-Based Scaffold

### Definitions of Mechanical Properties

Here we provide definitions of mechanical properties used throughout this patent. Mechanical properties include the following and closely related (with synonyms) terms: elasticity, plasticity, fragility, viscoelasticity, adhesiveness, stiffness, integrity, stress-strain curve.

The mechanical properties are used to describe the state of the tissues and their response to stress.

The stress is a force, pressure or shear applied to the tissue. The response is measured as a strain or deformation of the tissue. The various stress loads and corresponding strain values can be plotted against each other and are referred here as “stress-strain curve”.

A solid body is characterized as stiff if the strain-stress curve is steep, i.e. little deformation is tolerated. If the stress is higher than rupture barrier then the solid body breaks (loses cohesiveness or integrity) and body can be described as fragile.

A solid body is characterized as soft if the stress-strain curve is shallow, i.e. allows large strain (deformation).

A solid body is characterized as elastic when the deformation is reversible if the stress is removed. The stress-strain curve in case of elastic bodies have little or no hysteresis.

A solid body is characterized as plastic or gel-like when deformation is not reversible if the stress is removed. The stress-strain curve in case of plastic bodies have large hysteresis. Deformation of plastic (gel-like) bodies can also be tested in “creep test”.

### Mechanical Properties and Shape of Extracellular Matrix

Extracellular matrix plays an important part in providing a stiffness, shape, integrity, shape and architectural organization of mammalian tissues and consists of several proteins (collagens, laminins, perlecans, agrins etc) and proteoglycans (Aumailley et. al. 1998). Stiffness of extracellular matrix sets limitations upon organs ability to expand (e.g. cirrhotic liver), to move within certain range (e.g. adhesive capsulitis), to attain a different shape (e.g. wrinkles). Stiffness of extracellular matrix is capable of defining differentiation patterns of cells (Engler 2004, Cell), to stimulate or restrict migration of certain cell types.

Rigidity and shape is mainly maintained by collagen scaffold, which is formed by thick and dense cross-linked fibrils (Aumailley et. al. 1998). Crosslinking of collagen fibrils increases with age with tissues becoming more fragile and less elastic, as often seen in skin, eye lens, bones etc.

There are several types of collagen, which properties may differ, with some of the collagen types being tissue-specific. Tendon is composed of different molecules assembled into different architecture compared to pancreatic islet or cartilage.

### Architecutural Organization

Extracellular matrix provides not only durability and elasticity, but also architecture and compartmentalization of a complex tissue. For tissues like liver, pancreatic insulin-producing beta islets, ovaries and testis it is essential for their function.

### Highly Conserved Between Species

Extracellular matrix and collagens are conservative between species. That is why animal collagens are used in medicine. They have reduced risk of immune reaction and rejection.

### Highly Durable: Maintains Stiffness and Shape in Living or Dead Tissues.

Extracellular matrix and collagen scaffolds are extremely durable. Turnover of collagen fibrils in organism is extremely slow (years) compared to turnover rate of non-extracellular matrix molecules. Collagen scaffolds maintain their architecture, durability, shape and integrity for years, in living and non-living tissues.

### Mechanical Properties of Extracellular Matrix in Health and Disease

Mechanical properties of tissues are implicated in the following medical conditions

a) adhesive capsulitis (“Frozen shoulder”). Stiffness of a shoulder joint capsule with its ligaments, which limits the Joint Range of Motion (ROM) due to increased stress-strain characteristics of the diseased connective tissues (Lin, Hsu et al. 2008).

b) Dupuytren's disease. Palmar fascia gets thicker and shorter resulting in inability to fully extend the palm of the hand (Hurst, Badalamente et al. 2009).

c) phimosis is a condition where the foreskin cannot be retracted over the glans penis.

In the examples a), b) and c) the terms “stress”, “strain” and “stress-strain curve” can be interpreted in medical applications as follows:

Stress is a force applied to move the tissue. The strain is an actual tissue movement as a result of the applied stress. In treated tissues the stress-strain ratio is lower than in non-treated. It is either because less force is needed to move the tissue within the normal range, or because the range of movement is limited and forcing the tissue further is difficult and painful.

Treatment with TREZ would allow to soften the tissues and allow their faster remodelling and healing, while preserving a functional integrity

d) Fibrosis (liver, uterus, other organs) is an excessive formation and accumulation of fibrious tissues consisting of extracellular matrix as a result of reparative action. Fibrosis is most often seen in a liver (Wang, Palmeri et al. 2009) as a result of excessive consumption of alcohol or in hepatitis sufferers, but it also occurs in other organs (lungs—cystic fibrosis, intestine—Crohn's Disease etc). Tissue stiffness interferes with regeneration and expansion of tissues. Stiffness of liver precedes extracellular matrix accumulation; therefore, stiffness (increased stress-strain ratio) is not a consequence, but part of pathogenesis in liver fibrosis and cirrhosis (Georges, Hui et al. 2007).

e) Eye lens hardening. Flexibility of the eye lens is important for eye's ability to focus at various distances. Hyperopia (farsightedness) that often occurs at older age is related to lens hardening leading to the accomodative dysfunction (Hansen, Stitzel et al. 2003; Stitzel, Hansen et al. 2005).

g) Myocardial stiffness following infarction interferes with heart's normal function (Carroll, Janicki et al. 1989).

f) Stiffness of extracellular matrix is known to define cell behavior, such as: adhesion, migration, proliferation and even differentiation patterns. Modification of tissue stiffness in situ could be the key to regulate cell behavior, including differentiation, within natural environment of the cell (Discher, Janmey et al. 2005; Engler, Sen et al. 2006; Engler, Sweeney et al. 2007).

### Collagenolytic Enzymes in Medicine, Biotechnology and Industry.

Proteases are used in medicine, biotechnology, cosmetics, veterinary, food industry and other areas for decades (U.S. Pat. No. 3,003,917 by Beiler et. al., dated 1961). Examples include wound debridement (Klasen 2000), pancreatic islets derivation (Li, Yuan et al. 2009), cell culture (trypsin), proteolytical enzymes are used for meat softening, hide processing and other industries.

Collagenases are a subset of proteases that have collagen as a preferred substrate. We classify collagenases into following three categories

1. Bacterial collagenases—mostly pathogenic (reviewed Harrington, 1996 Infection and Immunity 64-6). Example: Clostridium histolyticum (gangrene)

2. Digestive collagenases from crab and shrimp (for example: one derived from hepatopancreas of crab).

3. Tissue-remodeling collagenases of vertebrate/mammalian/human organisms produced by host organism in order to remodel host's own tissues for healthy purposes, such as: organ growth, regenerative organ remodeling, reproduction-wise tissues remodeling.

Although these types of collagenases act on same substrate, their effect on the tissues is very different, which is most easily understood from their intended purpose.

### Bacterial and Digestive (Aggressive) Collagenases

Biological function of bacterial collagenases is to allow pathogenic bacteria to invade and spread into host tissue. Expression of bacterial collagenases allows pathogenic bacteria to pass through mammalian protection barriers in the body. As Clostridium bacteria (including Clostridium histolyticum) cause gas gangrene, the bacteria making collagenase don't care about maintaining health of the host organism. In our experiments we see that collagenase from Clostridium histolyticum digests mammalian tissues into microscopic, non-cohesive parts or to cell suspension.

Biological function of digestive collagenases (usually, of crab, shrimp or fish) is to digest any collagen-containing tissue into nutrients. Therefore, there is no biological reason for crab or shrimp to develop digestive collagenase that would preserve safe mammalian tissues and cells.

We suggest to unite all the collagenase that don't care for maintaining architecture and safe function of mammalian tissues under the name of aggressive collagenases. There is no evolutionary or physiological reason that those enzymes would preserve function, architecture and proper structure of mammalian tissues. On the contrary, aggressive enzymes are very efficient into digesting collagen scaffold into small, non-cohesive parts because that would fulfill the biological purpose of organism secreting those enzymes.

### Tissue-Remodeling Enzymes (TREZ)

However, nature created a number of proteolytical enzymes that can remodel mammalian (and other eukaryotic) tissues with high efficiency, however, being non-hazardous for the organism. Some of this enzymes are needed for organism growth (especially in embryonic and early postnatal age), some are needed for adult tissues remodeling (such as ovaries, uterus, mammary glands following onset of puberty, menstrual cycle, pregnancy, nursing following mammary gland involution), some are used by migratory immune cells to enable tissue-safe migration, some are overexpressed while organ repair after trauma (for example, skin healing or bone re-growth). All those natural processes require conditions named below:

(1) Re-structure (rearrange) extracellular matrix, especially rigid collagen scaffold, in order to make tissues soft, plastic, expandable stretchable and penetrable for cells, new-growing vessels, etc.,

(2) Maintain level of architectural integrity for affected tissues such, that it would allow the tissue to perform it's function while extracellular matrix is softened and rearranged. That would include, for example, cohesiveness of ligaments, intact structure of ducts within secreting organs, integrity of nerve axon bundles.

Expression and activity of tissue-remodeling enzymes, such as matrix metalloproteinases (MMPs), is part of natural, healthy self-maintenance of organism. Biological reason, therefore, proves safety and efficiency of such enzymes.

In mammalian (including human) organism such Tissue-Remodeling Enzymes would include a well-known family of matrix metalloproteinases (known as MMPs), and also less known families, such as: ADAMs, ADAMTSs, matriptases, collagenolytic cathepsins, collagenolytic members of kallikrein family and other endopeptidises, that possess protelytic activity towards extracellular matrix components that provide tissues stiffness. Some of those enzymes are proteolytic towards interstitial collagens (they are named collagenases), some towards collagen IV, some towards other extracellular matrix components. For example, native collagenases (proteolytical enzymes that are naturally responsible for digesting collagen structures in tissues during remodeling), would include gelatinase A (EC 3.4.24.24), neutrophil collagenase (EC 3.4.24.34), gelatinase B (EC 3.4.24.35), interstitial collagenase (EC 3.4.24.7), membrane-type matrix metalloproteinase-1 (EC 3.4.24.80), matrix metalloproteinase-11 (EC 3.4.24.B3), matrix metalloproteinase-13 (EC 3.4.24.B4), matriptase (EC 3.4.21.109), leukocyte elastase (EC 3.4.21.37), kallikrein 14 (EC 3.4.21.B45), cathepsin B (EC 3.4.22.1), cathepsin L (EC 3.4.22.15), cathepsin S (EC 3.4.22.27), cathepsin K (EC 3.4.22.38), meprin A (EC 3.4.24.18), matrilysin (EC 3.4.24.23). We include the list of individual TREZ enzymes in Table 1.

Since Tissue-Remodeling Enzymes have evolved to remodel tissues without harming them they have evolutionary developed properties that provide advantage over aggressive collagenases in such applications that require to maintain functional integrity within treated tissues.

Evolutionary-based functional difference between aggressive and tissue-remodeling collagenolytic and extracellular matrix-remodeling enzymes is reviewed in Table 2. We compare (1) bacterial, (2) digestive, (3) tissue-remodeling collagenolytic enzymes by set of criteria of biological reason for safety for mammalian tissues. We call “producing organism” such organism that has evolutionary developed the collagenolytic enzyme; we call “target tissue” the tissue that is biologically intended targed for the enzyme.

### Functional Integrity of Permanent Tissues

We introduce two key definitions: functional integrity and permanent tissues.

### Biological Function is Different for Proteolytical Remodeling of Permanent and Temporary Tissues

Tissue of our organisms can be divided into permanent and temporary. Permanent tissues maintenance is essential for (1) organism health and (2) survival of species, to which organism belongs. Examples of permanent tissues are: liver, kidney, pancreas, heart, muscle, nerve system, circulatory system, skin, tendon, cartilage. Lack or deficiency of permanent tissues is a disease (examples: lack of skin is wound or ulcer, lack of vasculature causes ischemia followed by necrosis, lack of liver or kidney causes systemic failure). We include reproductive organs (such as ovaries, uterus, testis, penis) because they are necessary for survival of the species, therefore, are permanent part of healthy organism.

Temporary tissues, such as fibrin clot, scar tissue or fat, are, as the definition implies, created to serve a temporary function (wound healing, blood clotting, energy storage) and intended later to be degraded by organism itself.

### Functional Integrity of Permanent Tissues

Definition: Functional integrity is level of tissue cohesiveness such that maintains the architecture integrity level sufficient for tissue to perform it's function.

For permanent organ or tissue to be functional (examples: glucose-dependent insulin secretion by pancreatic islets, for muscle to contract upon signal from nerves, for tendon to transmit muscle contraction effort to skeleton), several integrity requirements have to be fulfilled:

1. Maintain Tissue Structure and Compartmentalization

Permanent tissues evolved with a particular structure, tissue organization and compartmentalization to serve a biological purpose. For example, liver's vascular system, hepatocytes and immune system cells are strictly compartmentalized. In pancreatic islet insulin producing beta cells and vascular endothelial cells are proximal, but separated by a basement membrane. This arrangement is needed for a proper function of beta cells. In tendon the collagen fibrils are strictly organized to provide high level of elasticity and durability. Cohesion between collagen fibrils is essential for tendon durability and stress-resistance.

Blood-brain, blood-testis and blood-pancreatic barriers are examples of how compartmentalization is important. If these barriers fail, the immune system attacks brain, testis or pancreatic islets and destroys their function.

2. Maintenance of a Functional Cohesion Between Neighboring Tissues

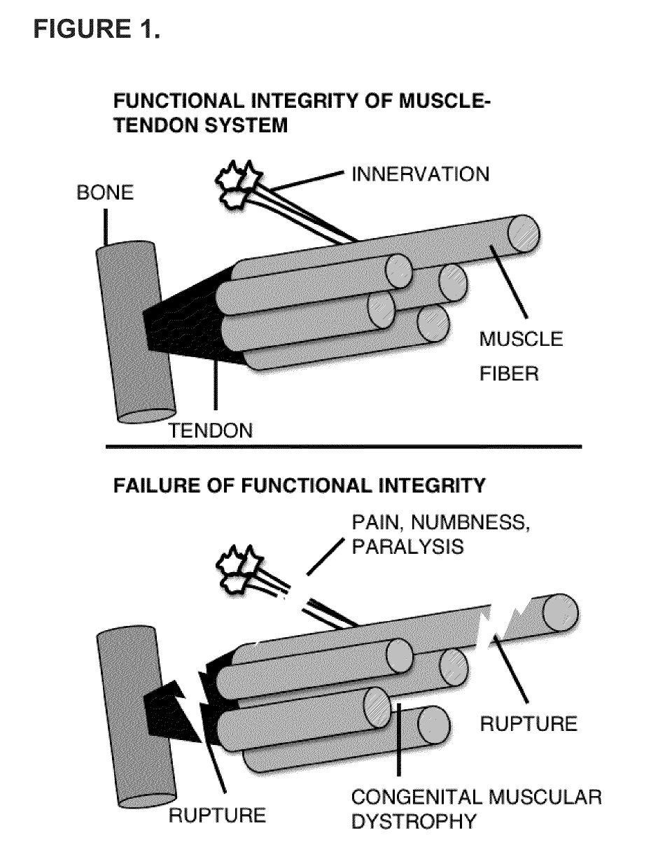

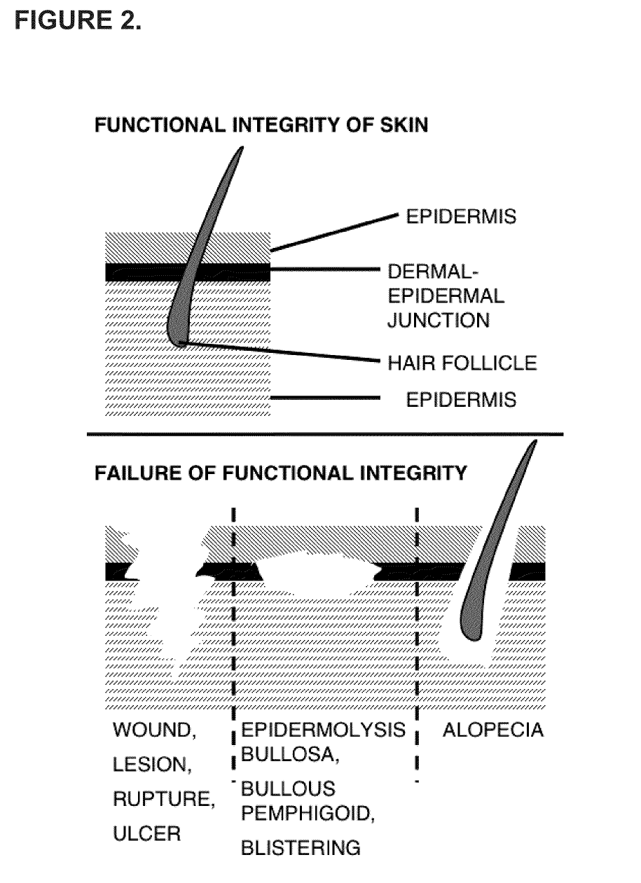

Functional integrity refers not only to a cohesion within tissue, but also how it connects to other tissues. FIGS. 1, 2 and Table 3 illustrate level required for functional integrity on example of muscle and skin. For example, if muscle maintains integrity and tendon maintains integrity, but muscle fails to connect tendon, then the whole muscle-tendon system fails to serve a biological function. If muscle fibers maintains integrity, but innervation within muscle is ruptured, the muscle will not able to react to neuronal signals and would fail the function. If skin dermis is detached from skin epidermis, it will cause blistering (example: epidermolysis bullosa, bullous pemphigoid, blistering)

3. Maintenance of Cohesion Between Cells and their Local Niches

Many cell types can function properly only if they receive proper signals from local microenvironment (cell niche). It is important that cell keeps contact with particular neighboring cells (same type or different type), extracellular matrix around and maintains a proper focal orientation.

### Nerve-Muscle-Tendon System: Functional Integrity

Functional integrity of organ and surrounding tissues is level of integrity and architecture maintenance that is essential for organism to perform it's function. Let's see how it works on example of muscle and tendon.

Function of muscle is (1) to receive signal from motor neurons, (2) contract upon the signal, (3) pull the tendons, (4) the tendons would move the bones and the organism would move. Additionally, (5) the healthy, undamaged muscle should not send “pain” signals via sensory neurons.

If any of the parts is impaired, it would jeopardize the organism function (see FIG. 1).

Therefore, the requirements for functional integrity for enzymatic treatment of tendon or muscle (for example, frozen shoulder, Dupuytren's syndrome):
1. enzyme should not disrupt the nerves (motor or sensory)
2. enzyme should not maintain connection of muscle fibers to one another
3. enzyme should not disrupt muscle fibers as such
4. enzyme should not disrupt connection of muscle to tendon
5. enzyme should not disrupt the tendon (tendon must retain connection between contracting muscle and skeleton)

### Biological Function of TREZ and Functional Integrity of Permanent Tissues

Nature needs TREZ for regenerative processes, for organs' development and growth during embryonic period, for tissue remodeling during reproduction, for migration of immune cells through connective tissues, for blood vessel growth in newly formed tissues. However, essential biological requirement is that during those processes all the permanent tissues must remain functional while treated by TREZ and afterwards; and, therefore must maintain functional integrity.

### Desired Outcome: Degradation or Cohesive Elastication of the Tissue

The desired outcome of enzymatic treatment of tissue can differ from complete tissue degradation to subtle change of its mechanical properties.

1. Areas that Consider Tissue Desintegration and Loss of Integrity as Positive Effect.

Desired outcome is degradation of extracellular matrix with release of cells or complete destruction of the tissue

Effect of tissue disruption or solubilization is desirable for purpose of the method. Highest speed and efficiency of tissue disruption is considered as criteria for method efficacy, not as hazard to safety and not as undesirable side effect.

### Examples

1. Single cell suspension from tissue or in vitro culture. Extracellular matrix has to be dissolved in order to free the single cells. The complete dissociation of tissue into single cellular level may be required for FACS or for cloning in vitro.

2. Necrotic wound tissue (in burn wounds and ulcers) debridement. Necrotic tissues are highly undesirable, as they spread spread toxins and pathogenic bacteria to underlying healthy tissues. The success criteria of debridement therapy is to dissolve or otherwise dissociate the necrotic tissue with highest rate and efficiency. The integrity of neighboring healthy tissues is important, but not that of a necrotic tissue.

3. Removals of scars. Scar serves as a protective temporary barrier in place of the wound, however rate of it's natural remodelling and clearance is very slow. Besides obvious cosmetic concerns there is a medical need to remove the scars that are painful, irritable or that interfere with normal functions.

4. Dissociate exocrine tissues of pancreas into microscopic particles in order to release endocrine insulin-producing beta islets from surrounding exocrine tissues. Insufficient dissociation of exocrine tissues makes islets retrieval more difficult; therefore, dissociation of exocrine tissues to few-cellular or single-cellular level is desirable.

Areas that Require Maintenance of Functional Integrity while Modifying the Tissue

Desired outcome: to soften and elasticate the tissue while preserving a functional integrity of the treated tissue/organ and surrounding tissues/organ. Loss of functional integrity by aimed tissue or neighboring healthy tissues is considered a side effect.

### Examples

1. Softening tissues in fibrotic organs (examples: liver fibrosis/cirrhosis, phimosis of penis tissue), however, organ should maintain proper architecture, compartmentalization, remain cohesive and preserve cell niches on microlevel.

2. Tissues for transplantation. The goal is to stretch them and adapt their shape for the recipient's lesion/wound shape.

3. Improve embryo implantation into uterus wall: uterine tissue must be soft and adhesive to attach embryo and allow it to invade uterine tissues, however, functional integrity and cohesiveness of uterine tissues is essential.

### Results

We have observed that the application of TREZ with various tissue types had one or more effects. In this section we describe the nature and mechanics of these effects (in terms of tissue's structure, integrity, mechanoelastic and viscoelastic properties) as well as their evaluation methods. Applications, such as surgery, transplantation, regenerative medicine, reproductive medicine, veterinary etc, including when combination of several effects results in additional benefit, are disclosed in a separate section.

### Functional Integrity

We observed with several tissue types that treatment with TREZ modifies tissue's mechanical properties, while the tissue preserves its functional integrity.

### Cohesiveness

The tissue samples, treated by TREZ, remained in one piece, unlike samples treated by bacterial or crab collagenase which dissociated into incohesive parts.

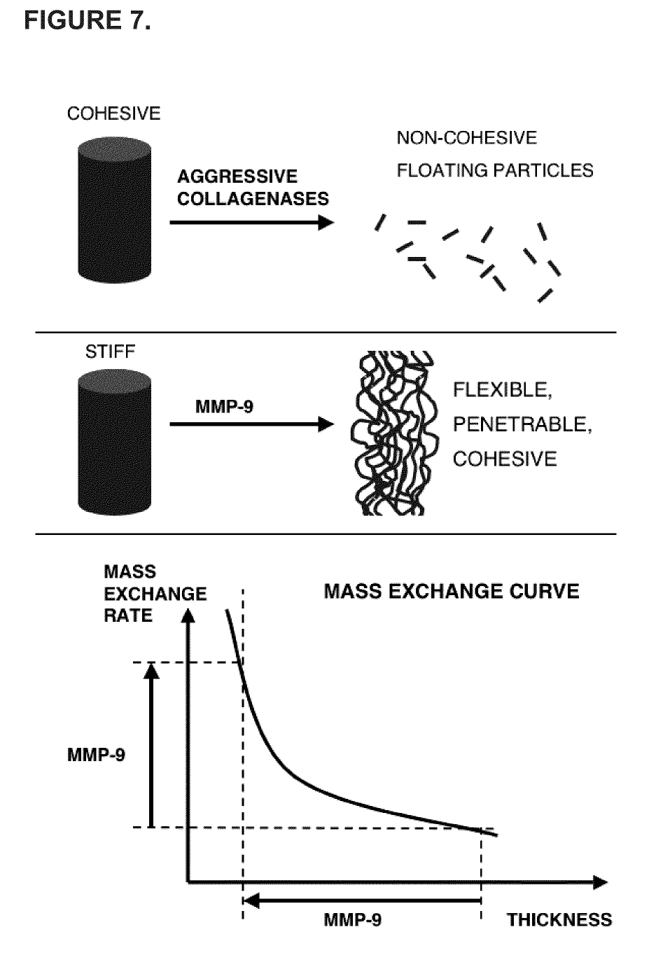

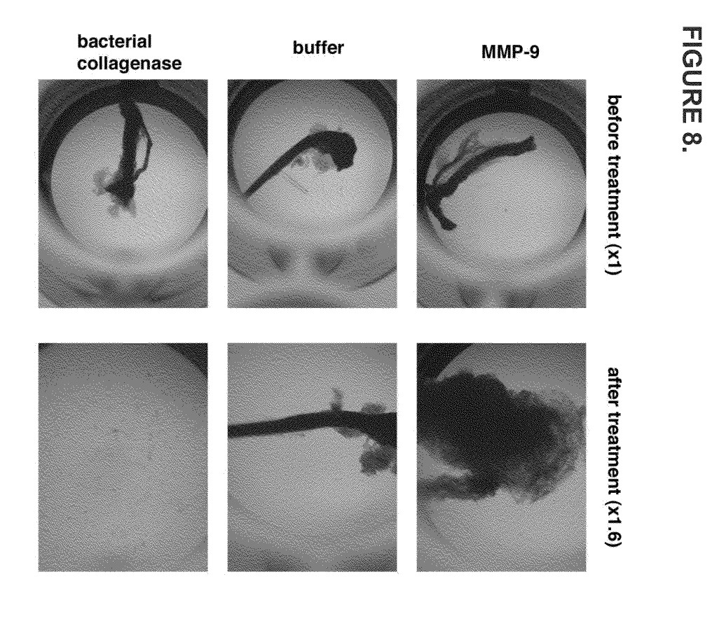

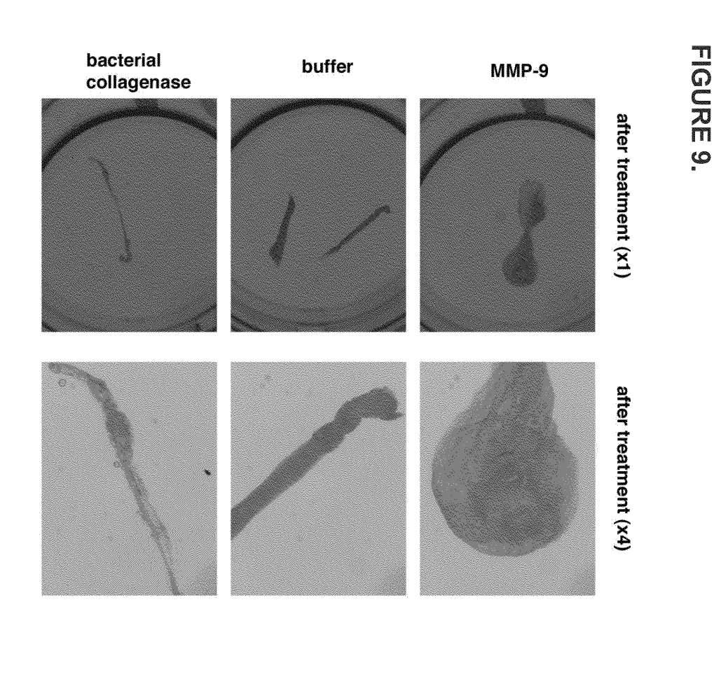

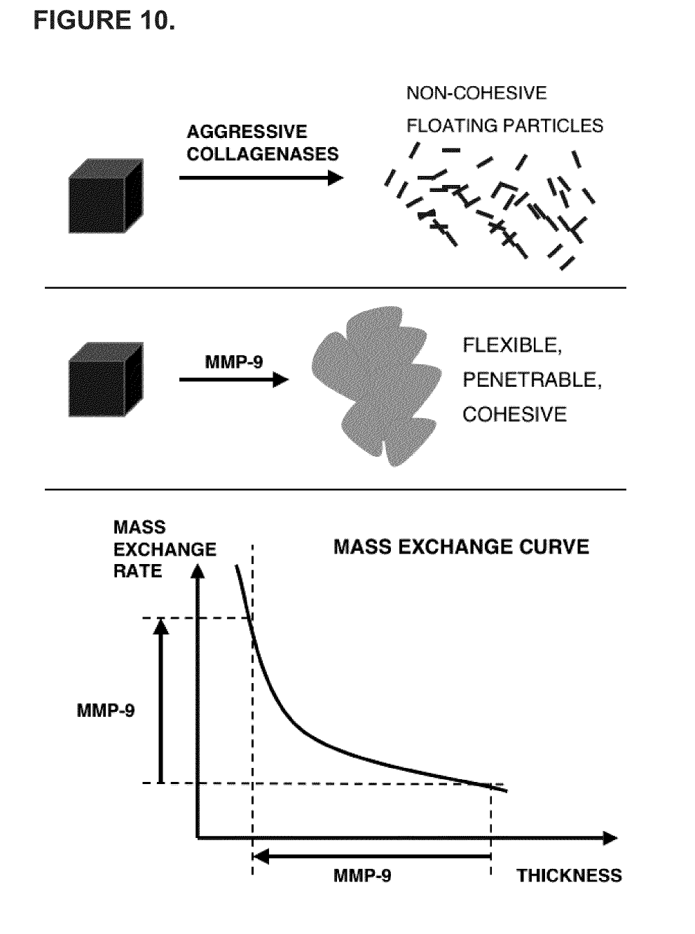

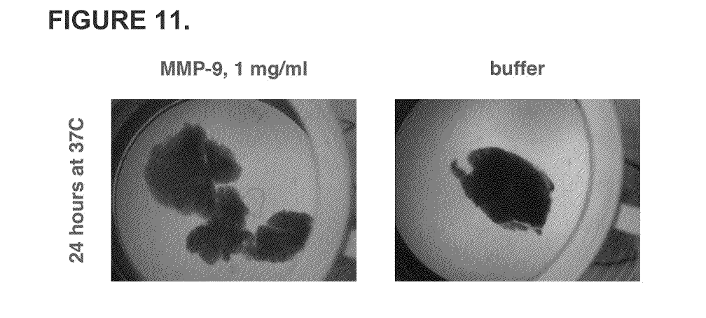

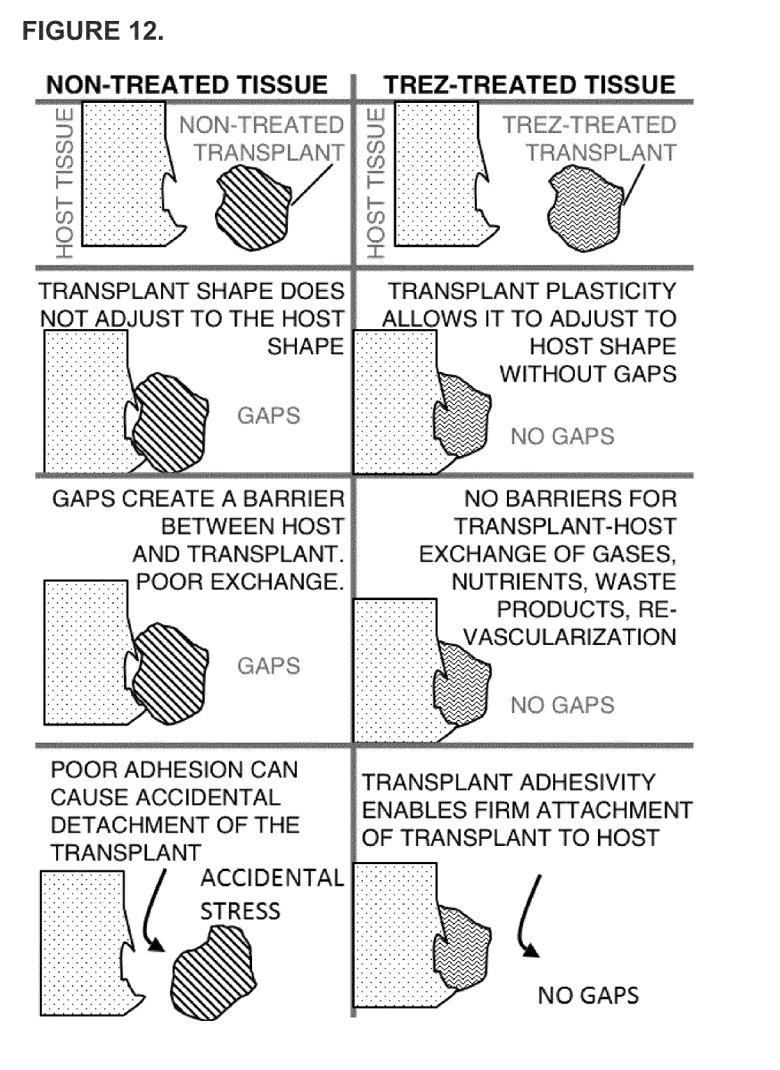

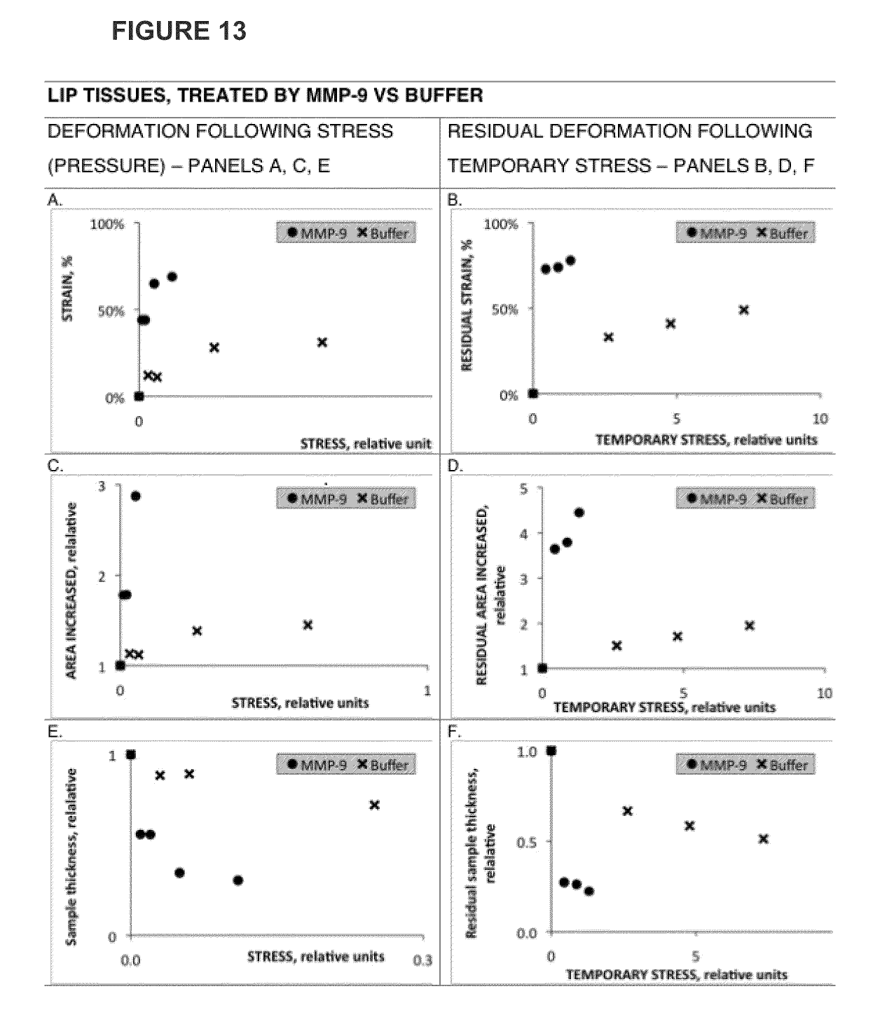

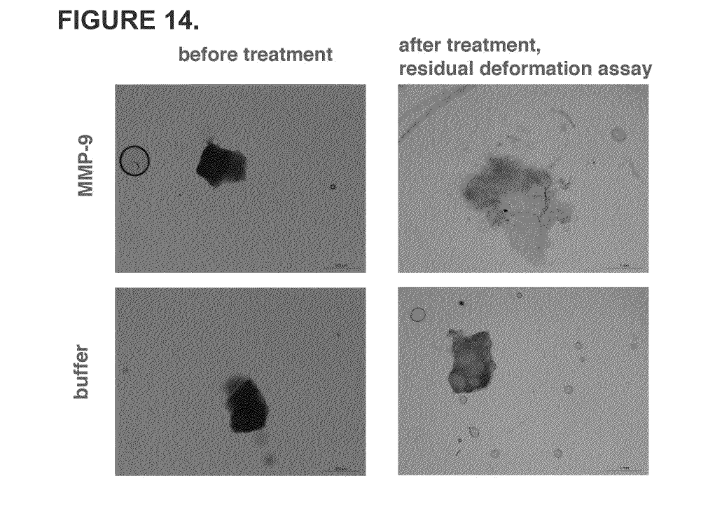

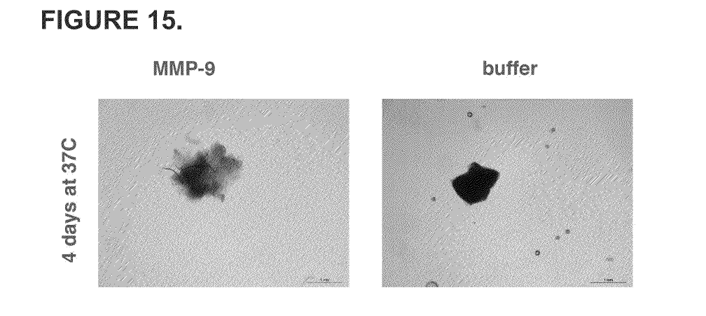

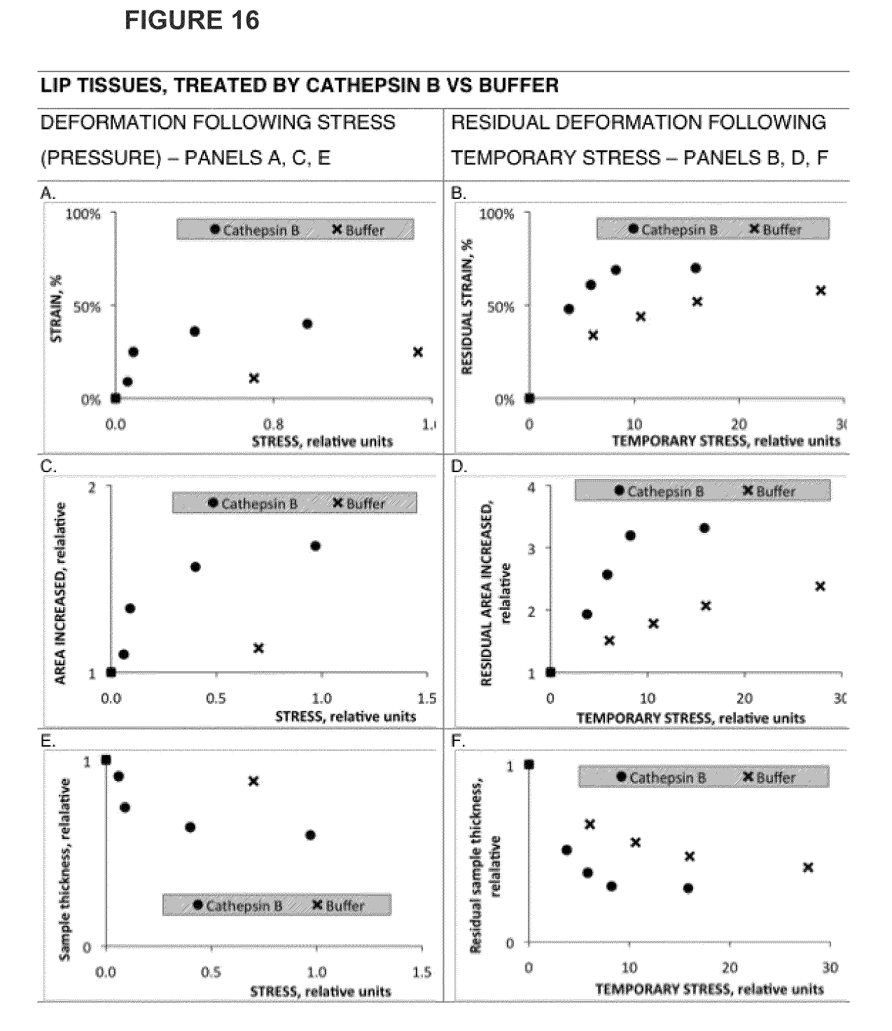

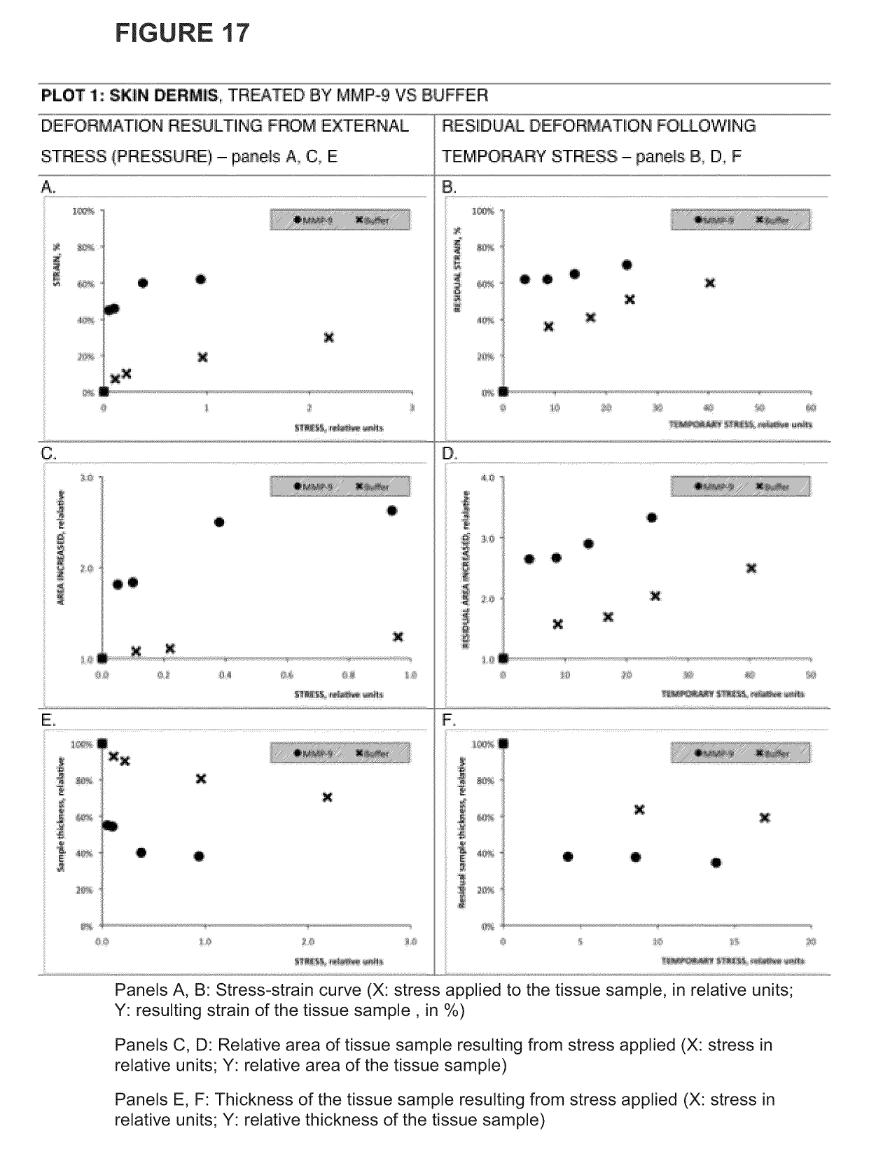

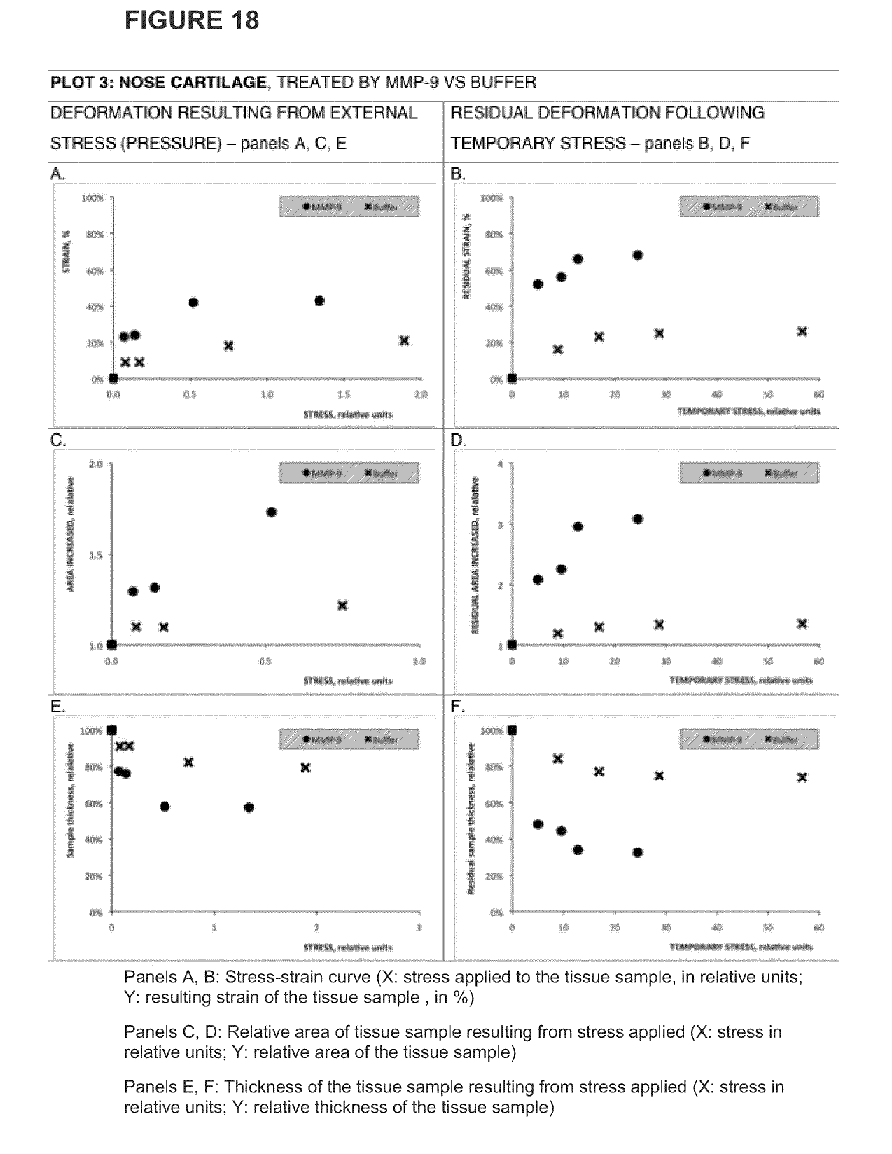

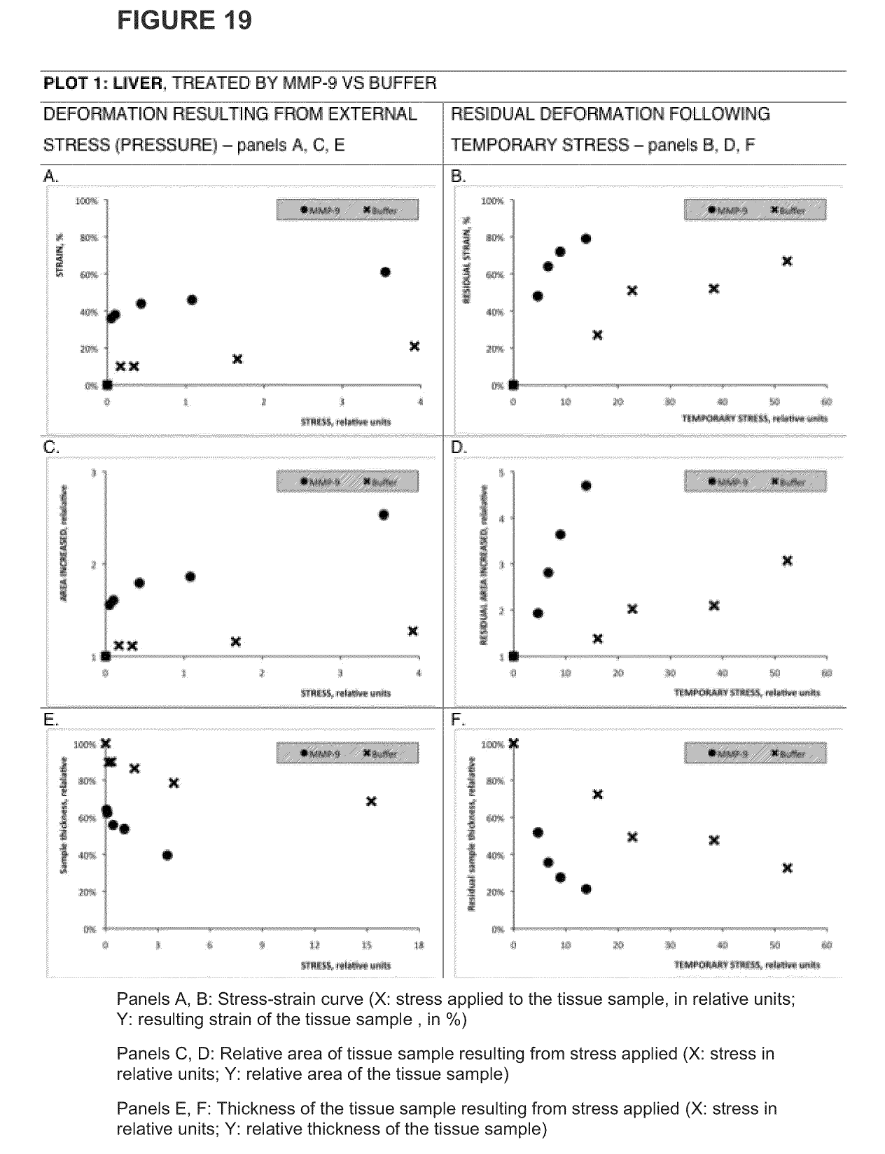

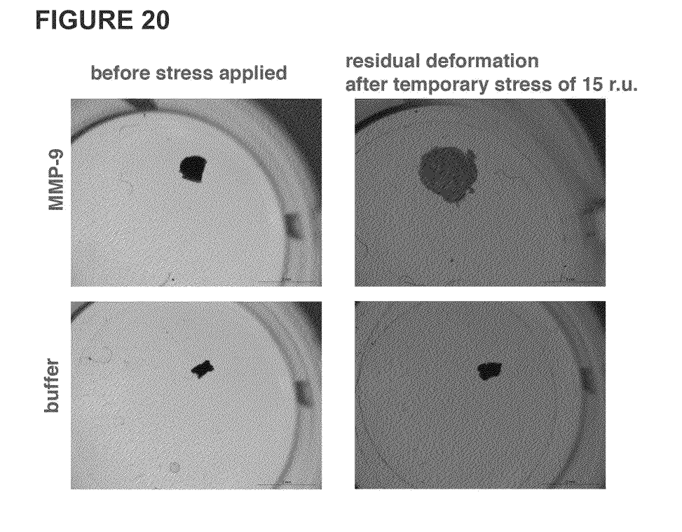

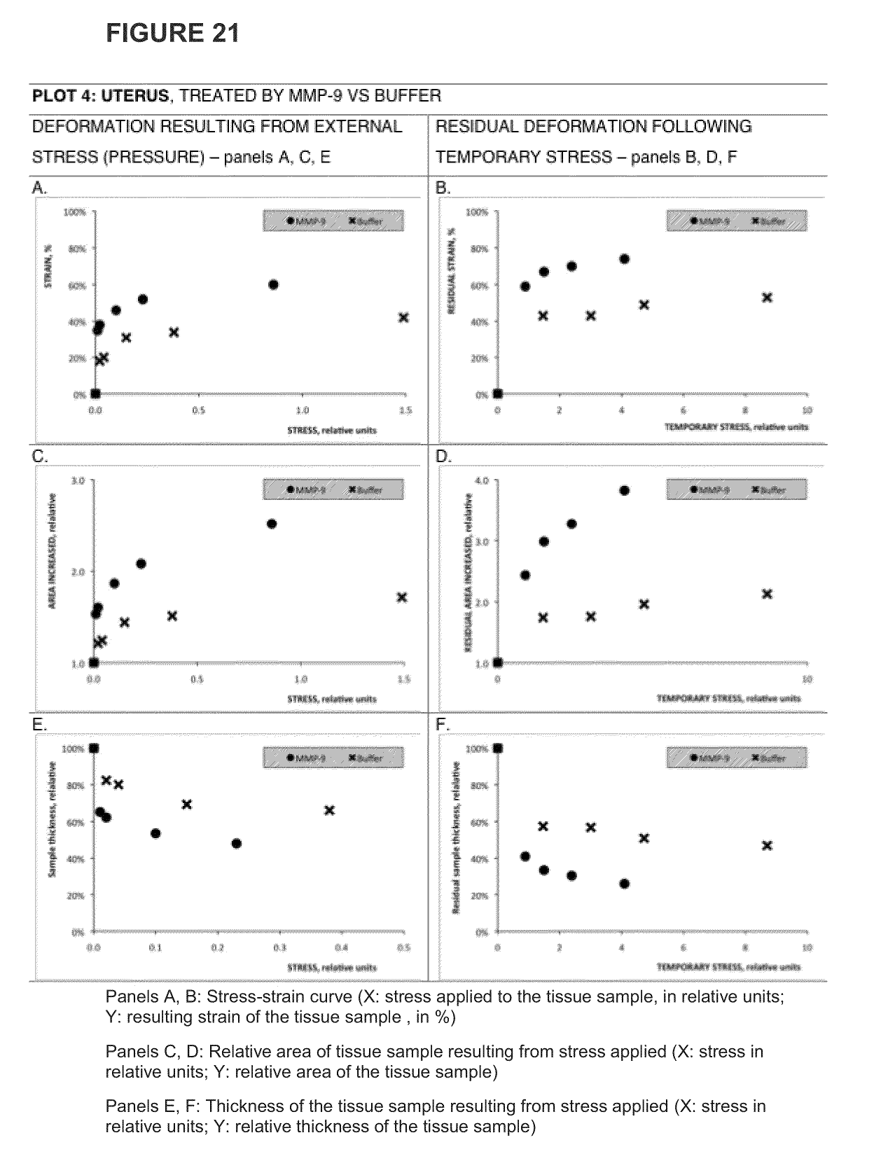

For example, MMP-9-treated tendon was split into individual, but interconnected fibers so that the tendon remains cohesive. Since the fibers remained intact and connected with each other and to the muscle, the functional integrity was preserved (FIGS. 7, 8, 9). In other example pancreas treated by MMP split into smaller lobules that are interconnected and have intact acini and intralobular ducts (FIGS. 10,11).

### Resistance to Rupture

Large-scale magnification phase contrast microscopy revealed that cells within treated tissues remain cohesive: no microscopic ruptures were observed to separate the cells from their niches. In fact, MMP-9 treatment even enhanced cohesion for cells within liver sample; treatment with MMP-9 rendered liver tissues less prone to cell detachment and to microscopic ruptures.

The cohesiveness of TREZ-treated islet at a cellular level was observed with a help of phase contrast microscope. The islet cells remained attached to their neighbors under increasing pressure load.

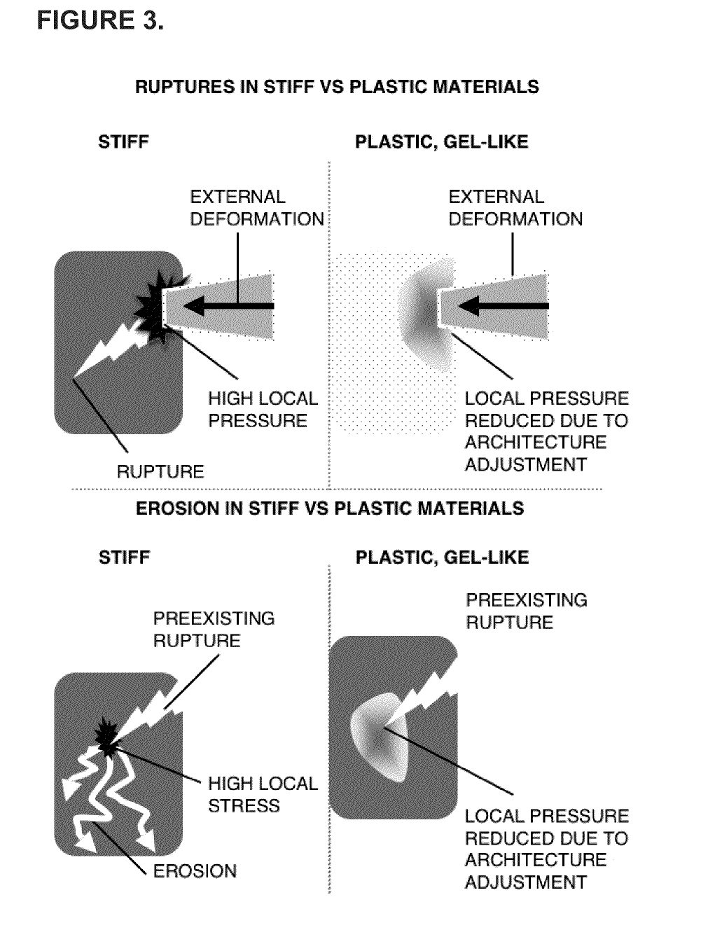

Resistance of TREZ-treated tissues to rupture and reduced brittleness (fragility) results from (1) increased plasticity and (2) reduced stiffness of TREZ-treated tissues. The mechanism of rupture- and erosion-resistance of TREZ-treated tissues is illustrated on FIG. 3.

### Functional Cohesion Between Different Tissue Types

Not only it is important to maintain cohesion between cells within one tissue type, but also to maintain such cohesion between neighboring tissues that is important for their function (for example: lack of cohesion between neighboring muscle fibers may result in dystrophia, lack of cohesion between dermis and epidermis may result in blistering, lack of cohesion between hair and skin may result in alopecia).

### Cohesive Under Stress Conditions

Tissues, treated by TREZ remained in one piece in the following conditions:

(1) suspended in buffer

(2) gently washed

(3) vigorously washed

(4) picked by forceps and hang in the air

(5) attached to adhesive surface and pulled by forceps

(6) subject to increasing pressure loads

Modification of Extracellular Matrix: From Stiff to Plastic (Gel-Like)

Permanent tissues are stiff and remember the original shape Some permanent mammalian tissues can be described as stiff (bones) and most others as elastic (muscle, skin, tendons)

In order to change shapes of permanent tissues methods of cosmetic surgery and reconstructive surgery are used. Shape of tissues can be modified by scalpel or surgeon or by application of long-term pressure (such as dental braces to correct positioning of teeth).

Many applications of regenerative medicine would benefit from tissues rendered more plastic, for instance to adjust the grafted material to a particular shape.

Plastic materials easily change shape and adjust shape to form to which they are placed, adhere to surfaces better.

### Rupture and Erosion of Stiff Materials Vs Plastic

As we discussed above, stiff and fragile tissues are prone to rupture and to erosion because little deformation is tolerated (see FIG. 3). Once ruptured tissues become susceptible to further damage by erosion at even lower stress levels. The mechanisms of plasticity including implications for processes of brittleness (fragility), rupture and erosion are reviewed and illustrated in (Picu R. C. (2011), Hong W. et. al. (2008), Wang X. et. al. (2012)).

As treatment by TREZ makes tissues more plastic (gel-like), they are less prone to rupture and erosion. Microscopic ruptures within gels can heal (Picu R. C. (2011), Hong W. et. al. (2008), Wang X. et. al. (2012)).

Permanent Tissues, Treated by Tissue-Remodeling Enzymes (TREZ) Modify from Stiff Materials into Gel-Like:

For many tissues we observed, that treatment with TREZ makes stiff, solid tissues more deformable, soft and adhesive, however they remain cohesive and thus maintain functional integrity.

### Creep Assays to Characterize Plastic Materials

Creep assay is used to measure how stress causes gradual deformation. The assay is carried out as follow. The tissue sample is placed on sensitive scale. A depth micrometer (Starrett 445MBZ-150RL Vernier Depth Gauge) is rigged on a stand above the sample. Micrometer screw applies a defined deformation to a tissue sample. The resulting stress is measured on the scale readout. Change of stress in time can be measured and recorded.

Modification of Extracellular Matrix: from Stiff to Elastic

TREZ-treatment allows to turn stiff tissues into stretchy We observed that treatment with TREZ allows to improve stretch abilities of treated tissues:
1. to increase area of tissue
2. to spread thinner the tissue sample
3. to stretch more when force is applied
4. less pressure is required to cause same stretch deformation
5. part of tissue sample can be picked by pincet, stretched out far and observed (the tissue remains cohesive)

### Assays to Qualify the Effect

The best assay to evaluate stiffness vs stretch ability of tissues is building stress-strain curve. The curve allows to estimate 1) Lower stress in TREZ-treated samples required to achieve a specific deformation (strain)

(2) Increased deformation (strain) in TREZ-treated samples with equal stress

However, we also used a set of correlating assays to qualify the effect.
1) stress-strain curve (quantitative, multipoint assay)
2) stress-relative area curve (qualitative, multipoint assay) qualitatively
1) whether tissue remained intact when picked by forceps and pulled
2) whether tissue remained intact when picked by two forceps from distal points and pulled apart
3) whether tissue sample became gel-like and filled the inoculation loop
4) whether it was possible to stretch the tissue on an adhesive surface

Reduce Brittleness (Fragility) of Fragile Tissues: Improve Range of Cohesive Elastic Deformation and Reduce Risks of Rupture and Erosion

### Tissues Fragility: A Risk for Rupture and Erosion

Many types of tissues loose their elasticity and become fragile as the person ages. The elastic tissue responds to stress with deformation and remains cohesive, while fragile tissue breaks (ruptures). Strain-stress curve can illustrate the phenomenon, where elastic tissue has lower stress-strain ratio, compared to stiff tissue. If the stress goes above the rupture barrier then the tissue breaks.

Important consequence of fragility is that, when once ruptured, it becomes more prone to damage (the rupture barrier declines). Plastic tissues are not as prone to erosion, on contrary, the rupture may even decrease with time, because highly plastic structures of gel-like structural organization have a limited capacity for self-healing (Picu R. C. (2011), Hong W. et. al. (2008), Wang X. et. al. (2012)).

### TREZ-Treatment Allows to Reduce Tissues Brittleness (Fragility)

### Examples

MMP-9 treated and buffer-treated eye lens were subject to same force compression. While buffer-treated eye lens cracked, the MMP-9 treated eye lens resulted to external force with elastic deformation, but not with rupture.

### Assays to Qualify the Effect of TREZ

The best assay to evaluate stiffness vs stretch ability of tissues is building a stress-strain curve. The curve allows to estimate the limits of how far the tissue can be compressed (before and after TREZ treatment). We demonstrate for many tissues stress-strain curves which indicate that TREZ-treatment allows to increase limits of cohesive deformation.

Another assay is to apply same force to equivalent samples of tissue, treated by TREZ or by buffer (negative control). Start with low force and gradually increase, equal for both samples. As TREZ-treated sample reacts with deformation, the untreated sample (or one treated by buffer) would remain stiff until it suffers rupture at certain load. We demonstrate the effect on tissue samples of (1) pancreatic islets, (2) eye lense (half), (3) eye lens (whole).

Improved Adhesiveness

Needs to improve tissue adhesiveness: surgery and transplantation

Normal tissues are not very adhesive to tissues or surfaces. However, tissues treated by TREZ become more gel-like and adhesive (1) to same type of tissue, (2) to different type of tissue, (3) to foreign surfaces.

However, increased adhesivity of mammalian tissues would be desired for medical applications, such as:
1) to allow better fit of transplant tissue to donor “wound” of complex shape
2) to allow wound borders fit closely, without gaps, in order to reduce risk of scarring
3) Adhesion would allow skin transplants to adjust to wound bottom of complex shape, thus enabling rapid exchange of gases, nutrients, waste products and enabling vascularization
4) Adhesion allows transplants or implants or wound borders to attach and hold firm. Not detach from each other by accident.
5) to improve embryo implantation to uterus

### Assays to Evaluate Adhesivity

Adhesivity is a consequence of tissue becoming more plastic. Besides plasticity assays there are qualitative assays to specifically evaluate adhesivity:
1) Does tissue sample adhere firmly to a inoculation loop? Can it be removed by forceps, or by washing with fluid?
2) Does tissue sample adhere firmly to a smooth surface (glass, plastic, etc)? Can it be detached by washing, gravity or centrifugation?
3) When tissue sample is placed on smooth surface, what is shape of meniscus between the tissue sample and the surface?
4) When the tissue is placed on same or other tissue, can it be easily detached by forceps or washing with fluid or centrifugation?

As we show for multiple types of tissue, treatment with TREZ significantly improved adhesive properties of tissues.

Shape Modification

### Need for Shape Modification

Possibility to change shape of permanent tissues would be highly desirable for (1) reconstructive surgery, (2) plastic surgery, (3) cosmetic medicine, (4) regenerative medicine, (5) transplantology and many other medical and veterinary applications (see FIG. 12 for illustration). Such applications would include (1) Peyronie's disease, (2) wrinkles removal and skin smoothening, (3) correction of teeth positioning of dental braces. The natural stiffness of majority of permanent tissue allows them to maintain shape within range of physiological range for many years; however, for named medical applications it would be desirable to increase temporarily plasticity of tissue and mold it into desired shape.

However, it would be desirable that a method to modify shape of tissues would not cause ruptures, blistering, ulceration, detachment of teeth from their sockets, pain and numbness, caused by rupture of nerves.

### TREZ-Treatment Allows to Modify Shape and Geometrical Proportions of Permanent Tissues

We have shown for many permanent tissues (for example: lip tissues, penis tissues, skin dermis and other tissues) that treatment with TREZ allows them to easily modify and easily retain desired shape. Importantly, that when stretched, compressed or otherwise modified, the TREZ-treated tissue samples remained cohesive: no ruptures occurred.

### Assays to Evaluate Shape Modification

The effect can be quantitatively described by the (1) plasticity stress-strain characteristics (described in previous chapter) and also by plots of: (2) area increase after temporary stress applied and removed and (3) reduction of tissue sample thickness after temporary stress applied and remove.

We also used several qualitative assays to describe ability of TREZ-treated tissue samples to change shape:
1) Is it possible to change shape of tissue sample by rearranging it on a coverglass?
2) Does the tissue sample attain the circular shape of inoculation loop, when placed within it?
3) Does the tissue sample mold into thin pipette tip, when placed inside it and subject to centrifugation?
4) Does the tissue sample retain it's original shape after 1 minute, 15 minutes, 2 hours, one day after temporary force was applied and removed?
5) Does the shape of tissue borders change from well-defined into diffuse and spherical?

Improved Permeability: Function-Wise Architecture Remodeling

### Need to Improve Permeability

Permeability is a key property to tissues viability and ability to expand.

Problem of tissues permeability appears in medicine and biotechnology when living tissues are separated from common blood circulation (in conditions such as: graft transplantation, islets for transplantation, ischemia, in vitro 3D tissue culturing). When thickness of tissue exceeds hundreds of microns and intrinsic blood circulation fails, the rate of gases, nutrients and waste products is reduced for cells within the tissue. Thickness of tissue lacking intrinsic blood circulation is critical for survival of cells within the tissue (Lehmann, Zuellig et al. 2007).

Effect of TREZ on tendon is shown on FIGS. 7, 8 and 9.

Effect of TREZ on pancreas is shown on FIGS. 10 and 11.

### Assays to Evaluate Permeability

Permeability is increased with (1) reduced thickness of individual components comprising tissue, (2) reduced stiffness and increased plasticity, that enables fluid flows within the tissue. On example of tendon and pancreas we can see such remodeling of tissue that allows to reduce thickness of individual components comprising the tissue many times. For many permanent tissues we demonstrated that TREZ-treatment allows to reduce thickness of the tissue sample significantly more, compared to tissues not treated by TREZ. Increased plasticity and decreased stiffness was quantitated and demonstrated for many permanent tissues.

### Practical Applications

In this section we describe how TREZ effect on permanent tissues may be used to solve particular needs of plastic surgery, regenerative medicine, transplantation, reproductive medicine, wound debridement, in vitro tissue culture, and other practical applications.

Transplantation, Surgery

### Needs in Transplantation

There in a number of needs that we aim to address by using TREZ on permanent tissues while maintaining functional integrity (see FIG. 12 for illustration):
1) Shortage of transplant material, therefore, need to stretch or expand
2) Need to reduce risks for transplant rejection; which includes: maintain viability of transplanted tissue and need to keep transplanted tissue adherent to host tissue
3) Need to maintain exchange of gases, nutrients and waste products (means: reduce risk for ischemia and necrosis) within transplanted organ; especially in case of grafts and islet transplantation
4) Need for host cells permeation, vascularization, innervation of transplant (dependent of tissue type)
5) Reduce risk of scarring
6) Reduce risk of pain or numbness or paralysis

### Skin Transplantation

There is need for skin transplantation, for example, after extensive burns. Autologous skin would be best solution, however, (1) transplant material is limited, (2) there is problem how transplanted skin graft would remain viable until it is re-vascularized on the new place.

As we show, application of TREZ (MMP-9 in our example) to tissues (including skin dermis) allows to soften, stretch, adjust shape, improve adherent abilities, while maintaining integrity. We also demonstrate same formulation of TREZ does not disrupt integrity of nerve bundle.

Consequences for skin transplantation could be:
(1) to stretch and expand dermis to further extent compared to non-treated tissues and remain whole. Thus, less donor material is needed to cover the wound area
(2) to reduce thickness of dermis tissue sample, thus improving mass exchange between transplant and host tissue
(3) to make dermis more plastic: allow it to adjust complex shape of wound borders, reducing risks or scarring
(4) to make dermis more plastic: allow it to adjust to complex shape of wound bottom, thus allowing close fit (no gaps between transplant and host) and improving mass-exchange, reducing risks for ischemia within transplant, and improving possibilities for vascularization and re-innervation of transplant.

We demonstrate experimental effect of MMP-9 to reduce stiffness, increase plasticity and adhesiveness of treated skin dermis (see FIG. 17; quantitative evaluation of the effect)

### Tissue Fusion (Cartilage and Other Soft Tissues)

U.S. Pat. No. 8,323,642 B2 and US 20080145357 A1 by Story et. al. “Tissue fusion method using collagenase for repair of soft tissues” addresses the need of tissues fusion on example of healing an artificial lesion within knee meniscus.

Authors use bacterial collagenase in order to release cells from collagenase-digested tissues adjacent to the injury site, thus migration of released cells would allow closure of lesion.

We suggest, that TREZ-softening of transplant and/or host tissue suffering lesion would aid the fusion process:
1) allow to maintain infrastructure, architecture and functional integrity of TREZ-treated tissue
2) allow to fit transplant to host tissues without gaps
3) allow to extend borders of lesion in order to reduce size of lesion or even to merge the borders
4) allow adhesion of connected tissue borders that allows to reduce risk of accidental opening of lesion.

We demonstrate experimental effect of MMP-9 to reduce stiffness, increase plasticity and adhesiveness of treated cartilage (see FIG. 18; quantitative evaluation of the effect). Effects for other soft tissues are shown on FIGS. 13-24.

### Islet Transplantation

Islet transplantation is extensively discussed below.

Expanding Tissues for Transplantation

There is a need to increase amount of transplanted material without loss of functional structure of it.

Most organs of human organism have strong capacity to expand. Adult stem cells can undergo numerous divisions and maintain ability (1) to proliferate, (2) to differentiate into functional cell types. Not only stem cells, but also differentiated adult cells have high capacity for proliferation, for example: pancreatic insulin-producing beta cells, vascular endothelial cells, fibroblasts, keratinocytes, hepatocytes.

However, the adult organs have very limited capacity to expand (much lower, compared to ability of cells to proliferate). In healthy organism limitation of organ growth by extracellular matrix stiffness is a natural mechanism to restrict size of our internal organs

In medicine there are situations when expanding organs, or tissues, or islets many-fold would be beneficial

Natural stiffness of extracellular matrix in adult organ limits ability of organs to expand. However, in embryonic state the extracellular matrix of organs is soft and allows the organs to expand rapidly without loss of function or architecture. We suggest that TREZ-softening of organs, tissues or islets aimed for transplantation would allow adult organs to expand in the way similar to expansion of embryonic organs.

We shall review several examples:

### Surgery: Flap Technique

Flap techniques allow to increase amount of autologous tissues for transplantation. The advantage of method is that tissue (while growing) is connected to blood circulation, thus reducing risks of ischemia.

It would be beneficial if tissues aimed for flap replacement would grow fast enough and in amounts sufficient to cover the wound/lesion.

We suggest that use of TREZ on tissues aimed for flap technique would allow:
1) the tissue to expand more rapidly (thus reducing time for painful flap therapy and hospital stay)
2) the tissue to expand (by organ proliferation) to larger size (thus, if lesion is large and flap material is scarce—like in lip reconstructive surgery after tumor removal), material could be expanded sufficiently to cover whole lesion
3) the tissue spreading area can be expanded also by stretching the softened tissue sample

### Liver

Living donor liver transplantation is a method to cure patients with end stage liver disease, such as cirrhosis and/or hepatocellular carcinoma (caused, for example, by hepatitis C or B infection). The liver is organ highly capable of regeneration; hepatocytes have high proliferation capacity.

We suggest that TREZ-softening of liver could be used to allow (1) the transplanted liver lobe and (2) the liver lobes left in the donor to expand to healthy amount of liver tissue and have no restriction to newly formed liver architecture caused by extracellular matrix stiffness.

We demonstrate experimental effect of MMP-9 on treated liver tissues (see FIGS. 19, 20).

### Surgery, Debridement

There is a need in surgery to remove necrotic tissues that are source of pathogens, can cause sepsis and inhibit natural healing processes. Examples: necrotic tissue removal after heavy burns, chronic ulcers.

Proteolytic enzymes (bacterial or crab digestive collagenases) are used to dissolve the necrotic tissues of burn wounds. Mechanical methods, such as surgery removal or vacuum pumps are also used for removal of necrotic tissues.

The desired outcome is to remove most of necrotic tissue without damaging a living tissue.

Problem is to separate the necrotic area from living area. (1) shape of damage is often of a complex form and thickness, (2) necrotic tissues are connected to living via common extracellular matrix, that is durable due to collagen network, (3) necrotic tissue must be removed fast enough before the sepsis starts.

We suggest that TREZ-softening of the damaged area, including healthy and necrotic areas, may be perform:
(1) softening of healthy tissues would not jeopardize their functional integrity
(2) softening of necrotic tissues would allow to remove them by mechanical means (examples: scalpel blade scratching, vacuum pump) without applying too high stress (that might damage the living tissues)
(3) softening of tissues would allow to pull them out and examine prior to surgical removal (scalpel cut). Thus in complex cases surgeon can decide on what depth to cut.

We demonstrate experimental effect a wide range of tissues: see FIGS. 13-24.

### Plastic Surgery/Reconstructive Surgery

Needs of plastic surgery and reconstructive surgery are:
1) to change the shapes of tissues (example: shape of nose, jaw, ear, facial tissues)
2) in case of cutting: to merge the lesion borders without gaps to avoid scarring
3) in case of injecting bioactive materials or scaffolds (like: collagen, hyaluronic acid, golden implants): to distribute the injected materials evenly, without bulges
4) to change positioning of organs/tissues (for example: teeth positioning in dentistry)

Ability of TREZ to soften the tissues without loss of functional integrity allows to serve those needs and to reduce risks of (1) tissue blistering or disruption, (2) complete loss of structure within treated tissue or neighboring tissues, (3) damage the nerve structure (thus causing pain, numbness or loss of motility). We suggest some examples of plastic surgery that can benefit from applying TREZ to tissues (but not limited to those named below):

### Peyronie's Disease

Pathology of Peyronie's disease is abnormal curvature of penis. Natural stiffness of penis tissues make it difficult to reshape it by purely mechanical methods. It would be desirable to make penis tissues more plastic in order to attain the penis a healthy shape, however, on condition that penis tissue would not suffer ruptures, blisters, disruption of connective tissues and nerves. As we show experimentally, MMP-9, being applied to penis tissues, improved their plasticity while maintaining them cohesive.

### Wrinkles Reduction in Cosmetic Medicine

Possibility to reduce wrinkles (smoothen surface of wrinkled tissues) is a much desired effect in cosmetic medicine. However, wrinkles are difficult to remove because, within physiological range, mammalian skin has low plasticity, therefore, skin “remembers” it's shape, which includes wrinkles.

We suggest that TREZ-treatment of wrinkled skin would allow to smoothen the skin and reduce wrinkles. As we show experimentally, treatment with TREZ improves plasticity of skin tissues, however, maintains functional integrity of skin (such as integrity of dermis, epidermis, dermal-epidermal junction and hair follicles). We suggest that procedure of plastic deformation of TREZ-treated skin into smooth shape would be accompanied by application of a durable scaffold of desired shape (example: golden threads technique).

We demonstrate experimental MMP-9 effect on treated skin dermis tissues: see FIG. 17.

Further embodiments include:

Abdominoplasty: Reshaping and firming of the abdomen

Blepharoplasty: Reshaping of the eyelids or the application of permanent eyeliner, including Asian blepharoplasty

Phalloplasty: Construction or reconstruction of a penis or, sometimes, artificial modification of the penis by surgery, often for cosmetic purposes

Mammoplasty, including

Breast augmentations: augmentation of the breasts by means of grafting, or implants.

Reduction mammoplasty: removal of skin and glandular tissue.

Mastopexy (“breast lift”): Lifting or reshaping of breasts to make them less saggy, often after weight loss (after a pregnancy, for example). It involves removal of breast skin as opposed to glandular tissue.

Buttock reshaping.

Buttock augmentation: enhancement of the buttocks using implants or grafting and transfer from other areas of the body.

Buttock lift: lifting, and tightening of the buttocks by excision of redundant skin

Labiaplasty: surgical reduction and reshaping of the labia

Reconstruction of vagina: Modifying geometrical parameters

Lip plastic surgery: after partial lip removal (for example, after lip tumor removal)

Lip reconstructive surgery, in case of cleft lip or deep wound

Lip enhancement: surgical improvement of lips' fullness through enlargement modification of lip shape

Rhinoplasty: reshaping of the nose

Otoplasty: reshaping of the ear, most often done by pinning the protruding ear closer to the head.

Rhytidectomy: (“face lift”): removal of wrinkles and signs of aging from the face.

Browplasty: (“brow lift” or “forehead lift”): elevates eyebrows, smooths forehead skin

Midface lift: (“cheek lift”): tightening of the cheeks

Chin augmentation: augmentation of the chin with an implant or transplant

Cheek augmentation: implants to the cheek

Orthognathic Surgery: Manipulation of the facial bones (see more in Bone Reconstruction chapter)

Fillers injections: collagen, fat, and other tissue filler injections, such as hyaluronic acid

Brachioplasty: Reducing excess skin and fat between the underarm and the elbow

We demonstrate experimental effect of MMP-9 on a wide range of tissues, including tissues of lip, cartilage of ear and nose, skin, penis, female reproductive organs on FIGS. 13-24.

Islet Transplantation

Donor-derived insulin-producing beta islets are successfully used for treatment Diabetes type I. Edmonton Protocol, currently used for treatment of diabetes patients, involves isolating islets from a cadaveric donor pancreas using a bacterial collagenolytic enzymes and injecting them into patient's portal vein aiming for attachment and hosting within patients liver. Several cadaveric donors are required to treat one patient. Efficiency of transplanted islets are several years.

There are several needs in area of islet transplantation into diabetic patients:
(1) Lack of human donor-derived islet material to provide help to all Diabetes type I patients.
(2) As several donors are required for transplantation, it is difficult to find all donors with same histocompatibility complex as the patient, therefore, problems with immune rejection and need for immunodepressants
(3) Human islets, due to size, after being extracted from pancreas, suffer ischemia due to size (in vitro storage of islets prior to transplantation)
(4) Poor survival rate of islets post-transplantation. Islets have problem with re-vascularization and re-innervation.
(5) Large size of human islets limits their ability to survive transplantation and function within patient after transplantation.

Using whole islets for transplantation has several advantages compared to transplantation of single beta-cells or beta-cell progenitors:
(1) pancreatic islet is a complex microorgan with function-wise cell compartmentalization
(2) architecture of pancreatic islets allows exchange of gases, nutrients and waste products between the endocrine cells within islet and blood circulation
(3) contact with blood circulation allows beta cells to detect level of glucose in blood and release insulin in glucose-dependent manner
(4) beta cells within islet form a syncytium structure, which enables all the beta cells within one islet to act in synergy.
(5) contact of insulin-producing beta cells with vascular endothelial cells, allows both of them to maintain functional phenotype

We have demonstrated that TREZ-treatment of pancreatic islets can allow to soften them and spread them thinner while maintaining functional integrity (without causing breakage (ruptures): see FIG. 23). Also we have shown, that TREZ-treated islets become more adhesive: they can merge with tissues without gaps, hold attached and contact flat or complex surfaces without gaps. Such effect could be beneficial for the field of islet transplantation in following ways (but not limited to those):

### Improve Exchange of Gases, Nutrients and Waste Products in Donor-Derived Islets (In Vitro)

Human islets, when derived from donor pancreases, suffer from insufficient exchange of gases, nutrients and waste products. In vivo the islets are surrounded by dense capsule, size of human islets varies within one to several hundred micrometers and mass exchange of endocrine cells within islets is enabled due to massive vascularization within the islet (a network of capillaries goes through each islet). After islets are detached from circulatory system, the inner capillaries of islet stop to serve function of mass exchange, and the only source of gases, nutrients and waste products is outside surrounding (in vitro culture medium or in vivo patient's bloodstream where the islets are injected).

Size of islets becomes a major problem in islet transplantation. (Lehmann, Zuellig et al. 2007) demonstrate that survival and post-transplantation function of human islets is in inverse proportion with the islet size.

We suggest that using TREZ on islets could aid the problem of size limitation on islet survival in following ways:
(1) modify shape of the islets, making them more flat (thus, improving accessibility of external molecules to the cells of the islet). Stretching of islets can be performed (for example): (1) by compression, (2) by adhesion to highly adhesive surfaces, (3) by centrifugation.
(2) allow to mechanically split the islets without ruptures into several cohesive parts, each maintaining local infrastructure (for example: by spreading and mechanically cutting into several pieces; by softening and mechanically splitting by sharp meshes, etc)
(3) by adhering the softened islets to adhesive surfaces, thus, reducing size of the islets.
(4) As islets become more soft and flexible, mass exchange can be enabled via flows of fluid, in the dynamic environment (for example: if the islet culture system is shaking or rotating or other way to enforce dynamic fluid flows around the islets)

Advantage of TREZ-softening is that modification of physical and geometrical properties of islet (softening, stretching, improved adhesivity, mechanically cut after softening) can be performed within few hours, before ischemia starts to affect the viability and healthy function of the islet cells.

### Improve Exchange of Gases, Nutrients and Waste Products in Patient-Transplanted Islets (In Vivo)

Islets, after being transplanted into the patient, face the same problem of insufficient mass exchange rate. It takes over several days till islets may restore inner vascular flow connected to the patient blood circulation. In current islet transplantation protocols there is a problem, that part of islets fail to survive and function within the patient body.

We suggest that use of TREZ could improve survival of transplanted islets by the same means as described for in vitro survival.

Additionally, we suggest, that as TREZ treatment makes the islets more adhesive and gel-like, that property may allow the islets to adhere better to patient's tissues (without gaps), therefore, reducing risk of rejection and improving re-vascularization of transplanted islets.

### Improve Banking (Reducing Size of Islets)

There is a need to have banking of donor-derived islets. The islets can be frozen and stored in liquid nitrogen, however, size of islets sets limitations on the procedure (freezing and thawing whole islets is a more complicated compared to single cells). Reducing size of islets (or thickness) could reduce the risks and complications of islets banking.

Islet size can be reduced by using TREZ as described in previous chapters.

### Improve Islet In Vitro Culture

Improving mass exchange within derived islets may be beneficial also for long-term in vitro cultures, for example (1) for scientists, working to understand islet function, (2) R&D specialists, working on ability to expand islets in vitro, (3) in drug screening, islet culture being a target for anti-diabetes drug candidates, (4) in drug screening, islet culture used to study side effects of potential drug candidates

### Enable Islet Expansion In Vitro

Possibility to expand insulin-producing pancreatic islets in vitro is a long-wanted need in islet transplantation and diabetes therapy. If islets could be expanded in vitro, (1) less of allogenic cadaveric donor material (pancreases) would be needed for transplatation, (2) it would be possible to arrange banking of all histocompatibility donor types, (3) it would allow to use a biopsy from living donor (for example: relative or histocompatible volunteer) to serve one patient instead of several whole cadaveric pancreases (as it is now).

We suggest that using TREZ-softening effect on islets could serve the purpose of islet expansion in vitro in following ways:
(1) Improve mass-exchange and, therefore, survival of the islets (as described above)
(2) Reducing thickness of the islet would make the cells within islet more accessible for hormones and growth factors, added to cell culture medium in order to enhance islet expansion and function
(3) Allow mechanical split of one large islet into several or multiple functionally-cohesive parts, each of size sufficiently small to allow healthy mass exchange and growth
(4) TREZ-softening of extracellular matrix within islet allows to reduce local stress in place of cell proliferation (effect, described in previous chapters). TREZ-treatment would allow to remove the tissue stiffness-resistance limitation on possibility of islet and beta cells to expand.
(5) Combination of TREZ-treatment and mechanical passaging techniques would allow to split (repeatedly) the expanded islets without disrupting the local cell niche contacts.

We suggest that described TREZ-softening techniques could be compatible with adhesive, 2D or floating, 3D culturing systems of pancreatic islets.

Enable Islet Passaging while Maintaining Functional Integrity.

As reviewed above

Tissue Fusion

Demands of tissue fusion procedure and advantages of TREZ-softening of tissues are described in previous chapter (1).

Including such applications as (but not limited to those named below):
tissues fusion in autotransplantation
tissue fusion in allotransplantation
tissue fusion in plastic surgery or reconstructive surgery
cartilage tissue fusion to fill cartilage lesions and cracks

We demonstrate experimental MMP-9 effect on a wide range of soft tissues: see FIGS. 13-24.

Reduce Risk and Extent of Scarring

Surgical procedures and accidental tissue ruptures result in scars. Examples:

post-surgery scars (examples: after Ceasarian section, after appendectomy), tissue ruptures (examples: torn wounds, female birthways are sometimes ruptured after baby delivery). Extent of scarring depends on ability of wound borders to merge without gaps between them. Sharp cuts, when closely merged together, result in thinner scars compared to torn wounds, where gaps between the wound borders are large. Quality of lesion healing is especially important in case of abdominal surgery.

Scar can be a cosmetic problem (like scars on face after facial plastic surgery), distort mimics (scar on lips), distort sexual life (scars formed after rupture of birth ways, caused by traumatic delivery), distort vision (scars on eyes). Some people are prone to extensive scarring; reducing risk of scarring would be beneficial for them.

We suggest that use of TREZ on permanent tissues would allow to reduce risks and extent of scarring. The mechanism, as described above:
1) to adjust shape of lesion/wound borders to merge without gaps
2) to stretch and extend softened borders of lesion or wound enough to cover the lesion/wound gap
3) to allow better adhesion of softened, gel-like lesion borders
4) softened, well-adhered lesion borders (after connected) would be less prone to accidental opening.

We demonstrate experimental effect of MMP-9 on skin, lip tissues and other soft tissues: making them more adhesive and allowing gapless adhesion to similar or different surfaces.

Reduce Risk of Rupture and Erosion

Previously we described how TREZ-softening of tissues would make tissue less prone to rupture and/or erosion. The effect can be used in many aspects of surgery, treating fragile skin prone to rupture and erosion, etc.

Use of TREZ could aid to serve following needs:

### Reduce Risk of Rupture or Erosion

Living tissues sometimes suffer risk of accidental deformation, compression, tension that can cause high local stress and, therefore, local microscopic ruptures. In fragile tissues microscopic ruptures may undergo subsequent erosion and result in major ruptures.

As TREZ-treatment renders tissues more soft and gel-like, it would allow (1) to reduce local stress, (2) reduce risk of microscopic ruptures, (3) reduce risk of erosion for already occurring microscopic ruptures.

### Heal Rupture or Erosion

If tissues already suffer rupture or erosion, use of TREZ on ruptured tissue could (1) enhance ability of rupture to heal partially or completely and (2) reduce risk of increasing rupture as a result of accidental stress. The mechanisms are described above and are similar to ones described for “reducing risk or scarring” and “tissue fusion”.

We demonstrate experimental MMP-9 effect of reducing brittleness and therefore reducing risk of rupture for (1) pancreatic islets, (2) eye lens: see FIGS. 23 and 24.

Orthopedics and Bones Reshaping:

Skeleton (bones) structure and shape define shape of our body. As bones survive strong tension and compression, they are extremely durable and keep shape.

However, there is sometimes a need to change geometrical size and shape of bone, or transplant bone material.

Rigidity of bone is enabled by mineral structure, however, elastic properties and ability to survive tension or compression or deformation without ruptures is due to extracellular matrix (collagen-based architecture).

We suggest that TREZ-softening of demineralized bone could aid different areas of orthopedics:
1) render demineralized elastic bone matrix softer and, therefore, expandable
2) render demineralized bone matrix softer, therefore, reduce extracellular matrix resistance to penetrating cells, vascularization, osteogenic processes.
3) Maintain integrity and original complex architecture of bone
4) Reduce risk of scarring in case of fracture, lesion or transplantation
5) Enable better fit of transplant tissue to host tissue, therefore, improve implantation

Distraction Osteogenesis (Limb Lengthening)

Distraction osteogenesis (synonyms: distraction callotasis, and osteodistraction) is a surgical process used to reconstruct skeletal deformities and lengthen the long bones of the body. Recent methods involve bone fracturing, following procedure of slowly moving apart two bone ends in order to allow formation of new bone within the gap. Modern methods involve: use Ilizarov apparatus, the Guichet method, intramedullary skeletal kinetic distractor, fitbone surgery.

Conditions, that would benefit from limb lengthening procedure, include: congenital deformities (birth defects), developmental deformities (neurofibromatosis, bow legs), post-traumatic injuries, bone infections and diseases, post-tumor removal, short stature.

We suggest that use of TREZ-softening combined with bone demineralization could make limb lengthening procedure (1) more easy, (2) more effective, (3) allow faster results.

### Facial Plastic and Reconstructive Surgery

Reconstructive surgery of face bones allows (1) to change the shape of face for cosmetic purposes, (2) to restore face bones shape and function in case of facial bones damage.

Method of fracturing bones is especially traumatic and risky, when it concerns

### Spinal Disc Damage

Spinal disc herniation (prolapsus disci intervertebralis) is a medical condition affecting the spine due to trauma, lifting injuries, or idiopathic (unknown) causes, in which a tear in the outer, fibrous ring (annulus fibrosus) of an intervertebral disc (discus intervertebralis) allows the soft, central portion (nucleus pulposus) to bulge out beyond the damaged outer rings.

U.S. Pat. No. 7,935,337 by Haro suggests use of MMP-3 and MMP-7 to reduce amount of tissue for herniated spinal (intervertebrate) discs. However, authors aim for degradation and resorbtion of herniated spinal disc tissue. We suggest use of TREZ to be applied to herniated disc tissue, however, aiming for maintaining functional integrity of spinal disc tissue (effect opposite to degradation and resorbtion). We believe that functional integrity of spinal disc tissue and adjacent tissues is essential for healthy connection of spinal disc (while degradation of spinal disk tissue may result in atrophy of soft tissues and, therefore, vertebrae trauma). We suggest, that herniated disc, softened by TREZ and remaining functional integrity, can be treated by:
(1) changing shape and geometrical properties of intervertebrate disk, by application of TREZ in composition that enables functional integrity of vertebrate disc and adjacent tissues.

### Plastic or Reconstructive Surgery

Plastic or reconstructive surgery of bones can benefit from use of TREZ as described above.

### Regenerative Medicine

Regenerative medicine offers opportunities of creating artificial tissues using (1a) autologous patient's cells (adult cells, stem cells, iPS cells or else) or (1b) allogenic donor compatible cells and (2a) artificial scaffolds, (2b) patient's own extracellular matrix, (2c) allogenic or xenogenic extracellular matrix. Another option is expanding tissues (autologous or donor) in vitro to increase amount of transplant material. In future, regenerative medicine approach could provide advanced technologies on orthopedics and bone reconstructive medicine.

We suggest that use of TREZ on decalcified bone tissues could benefit the regenerative technologies for bones reconstruction in mechanisms described above. In further chapters we shall describe potential benefits of using TREZ for softening of allogenic or xenogenic extracellular matrix material, devoid of cells.

Reproductive Medicine and Fertility

Many fertility problems can be related to increased stiffness of uterus. Embryo implantation involves process of early embryo invasion within uterine tissue; process depending on the ability of uterus for plastic deformation. Exchange of gases, nutrients and waste products between placenta and uterus may also depend on mechanoelastic and viscoelastic properties of uterine tissues. Method to improve mechanoelastic properties of uterus, however, require condition of maintaining uterine tissues cohesiveness. Loss of functional integrity, such as ruptures or desintegration of uterine tissues may result in placenta detachment, spontaneous abortion, uterus ulceration and scarring.

### Embryo Implantation,

Success of embryo implantation depends on ability of early embryo (blastocyst) to penetrate within uterine tissues. Pathological stiffness of uterine tissue may hinder embryo penetration, therefore, hinder fertility. TREZ-treatment of uterine tissues may resque possibility of uterus to accommodate the early embryo.

Medical cases, where TREZ-softening of uterine tissues may be beneficial:
(1) aid natural implantation, where uterine tissues are too stiff to enable embryo adhesion and embryonic invasion
(2) in vitro fertilization cases, to enable implantation of in vitro-formed embryo

### Placenta Formation and Improved Function

Uterus wall softening may allow better placenta adhesion to a uterus wall. Treatment of placenta and/or uterus wall make tissues more permeable improving gas exchange, vascularization, improving access to nutrients and removal of waste products

### Re-Attachment of a Partially Detached Placenta

Partial detachment of placenta carries a risk of spontaneous abortion, ischemia and permanent damage to the fetus. Therefore, there is a need to improve adhesive properties of placenta and uterus in order to restore proper exchange of gases, nutrients and waste products between fetal and maternal organisms.

Partially detached placenta can be treated with TREZ thus rendering the placenta and uterine wall more adhesive and plastic. This enables re-attachment of placenta to uterus by strong adhesion without gaps

### Uterine Surgery

Surgery of a uterus (any cause e.g. because of Cesarian section, injuries, reconstructive surgery, ulcer or cancer) may leave scars that impair a normal function of the uterus during pregnancy

We suggest that procedure of (scar, malformation) removal may be combined with treatment by TREZ-treatment of lesion borders. Resulting higher tissue plasticity and adhesiveness would result in better tissue healing without scars and better organ function

### Prenatal Diagnostics

Sometimes there is need to perform early embryo diagnostics to identify potential genetic abnormalities. The aim is to detect birth defects such as neural tube defects, Down syndrome, chromosome abnormalities, genetic diseases and other conditions, such as spina bifida, cleft palate, Tay Sachs disease, sickle cell anemia, thalassemia, cystic fibrosis, Muscular dystrophy, and fragile X syndrome.

Invasive techniques (like, biopsy of embryonic tissues being obtained for analysis) allow to analyze genetic status of the fetus, however, they have a risk of damaging the fetus or placenta or cause spontaneous abortion.

We suggest that preliminary TREZ-softening while maintaining functional integrity may reduce risks of occasional lesion, rupture or erosion caused by invasive techniques.

### Cervix Softening/Ripening to Induce Labor

Cervis softening (ripening) is a natural process to enable baby passage out of uterus while delivery. In nature the cervical tissues attain abilities to stretch before labor. However, sometimes the cervix is stiff and does not open enough.

We suggest that use of TREZ may be benefitial:
(1) to soften the stiff cervical tissues during the labor or shortly prior to it
(2) to soften cervical tissues to reduce risk of rupture during the labor
(3) if rupture occurred during labor, to use TREZ in order to reduce risk of scarring and improve rupture recovery (as described above)
(4)

### Softening Birth Ways (Cervix, Vagina, Vaginal Opening, Labia, Perineum)

Delivery can sometimes be traumatic for female birth ways and adjacent tissues. If baby size is large, or tissues are stiff, the tissues may suffer ruptures.

We suggest that TREZ softening could be beneficial
(1) to soften the stiff tissues during the labor or shortly so that they would be able to undergo wide range of deformation without suffering rupture (or to reduce extent of rupture)
(2) if rupture occurred during labor, to use TREZ in order to reduce risk of scarring and improve rupture recovery (as described above)

### Post-Delivery Ruptures to Reduce Risks of Scarring

Birth ways may suffer ruptures during delivery.

If untreated, the ruptures would heal with extensive scarring. It may cause female pain (including during sexual life) and become a problem for following deliveries.

(1) We suggest, TREZ-softening may be applied before delivery to reduce risk of ruptures

(2) TREZ-softening may be applied to ruptured tissues of vaginal opening, labia, etc. in order to improve healing (enable close fit of ruptured tissues and reduce risk of scarring)

We demonstrate experimental MMP-9 effect on treated tissues of uterus and ovaries: see FIGS. 21 and 22.

Ophtalmology

Eye is a complex organ. Though ability to change physical and/or geometrical properties of certain eye structures could be beneficial for some ophtalmological conditions, it is essential that all the eye structures maintain functional integrity, architectural organization and cohesiveness during the treatment.

### Optical Properties of the Eye

Optical properties of the eye depend on (1) geometrical shape of eye lens, (2) eye lens stiffness. Stiffness of eye lens defines range of accommodation that defines range of focused. Age-related increase in stiffness results in decreased ability of eye accommodation and reduced range of focused vision. Age-related eye tissue stiffness results in increased fragility, therefore, increased risk of accidental eye trauma (Hansen, Stitzel et al. 2003; Stitzel, Hansen et al. 2005).

### Eye Lens Stiffness and Fragility

Possibility to reduce stiffness and reduce fragility of aged eye lens by application of TREZ to stiffened eye lens would allow:
(1) to improve ability of aged eye for accommodation
(2) to avoid risk of eye trauma, caused by eye tissues fragility

We demonstrate experimental MMP-9 effect of reduced eye lens brittleness: see FIG. 24.

### Corneal Transplantation (Expand Tissue)

There is a need for corneal transplantation in case of corneal trauma (example: burns).

We suggest that TREZ-treatment could serve same beneficial purpose as described for other transplantation cases.

### Surgery of Eye

Surgical operations are performed on eye. However, there is a risk of scarring. Particularly for the eye, the scar not only affects appearance and causes irritation, it also affects the ability for vision.

The use of TREZ in order to reduce risk of scarring may be applied to applications of eye surgery.

Decrease Stiffness of Pathologically Stiff Tissues

A number of diseases are direct result of increased stiffness of organs. Increased stiffness may (1) decrease body motility range or (2) decrease ability of organ for regeneration.

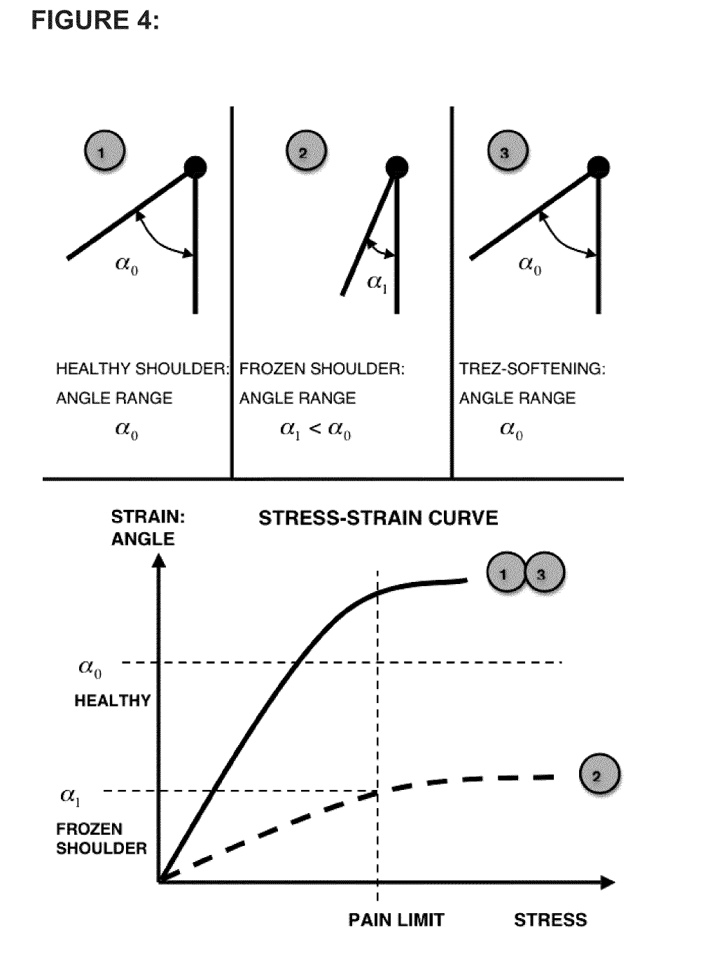

Dependence of pathology from stress-strain (stiffness) and geometrical deformation range characteristics is reviewed on Table 4 and FIGS. 4,5.

### Dupuytren's Contracture

Pathological condition of Dupuytren's contracture results from increased stiffness and contracted shape of Dupuytren's cords. Therefore, the contracted finger has reduced range of motility: see FIG. 3 of (Hurst, Badalamente et al. 2009) for detailed illustration. Collagenase from Clostridium histolyticum, when applied to Dupuytren's cords in vitro allowed 93% decrease in tensile modulus and 3,600 units dosage; however, only 300 units dose of collagenase was sufficient for cords rupture within physiological cords limits (Starkweather, Lattuga et al. 1996). Collagenase of Clostridium histolyticum when applied to Dupuytren's cords in vivo was shown to restore motility of finger within healthy range; however, the method was shown to cause a wide range of side effects due to loss of functional integrity of nerves, connective tissues or finger tendon: see Table 3 within publication of (Hurst, Badalamente et al. 2009).

In our experiments we demonstrate a different approach: we suggest that TREZ-treated tissues may be not digested, but (1) made more elastic and stretchable, (2) made more plastic to increase length, however, (3) without loss of functional integrity for treated and adjacent tissues (including tendons and nerves).

### Phimosis

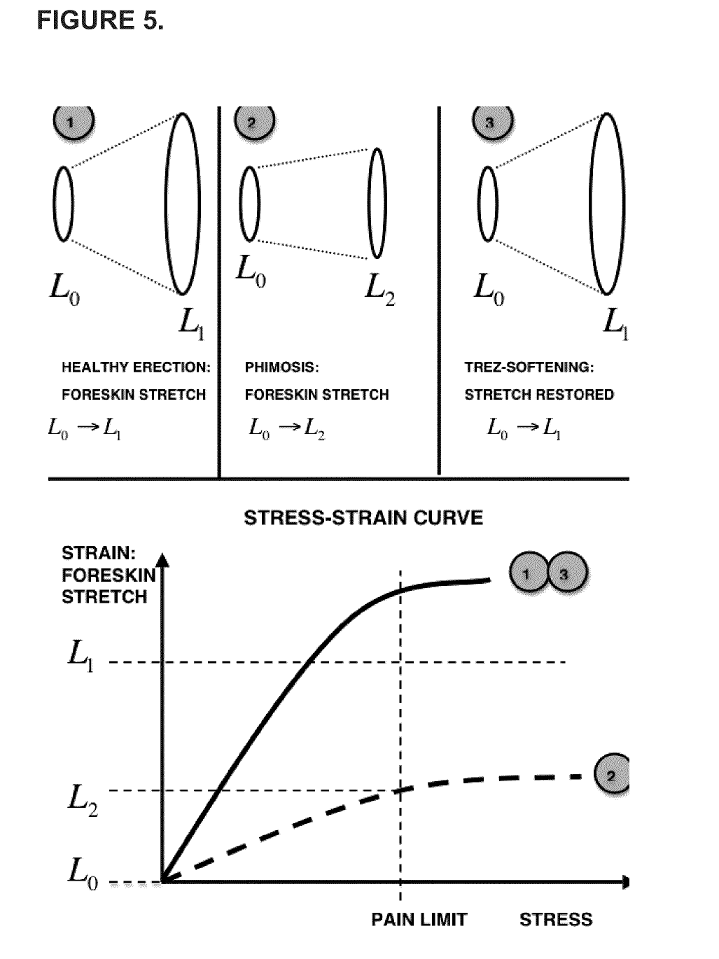

FIG. 5 illustrates, how pathologically increased stress-strain characteristics (increased stiffness) of penis connective tissue reduces it's possibility to stretch within healthy range, thus causing pain and disfunction. Use of digestive collagenase has limitations, as partial digestion and disruption of penis tissues would result in severe side effects.

We suggest that using TREZ on stiffned tissues of penis foreskin of phimosis-suffering patients can restore healthy range of deformation. We have demonstrated that MMP-9 treated penis tissues (foreskin and connective tissues) show reduced stiffness and ability to stretch, while maintaining cohesion. Importantly, in our experiment the penis tissues and control nerve samples, when treated by functional dose of MMP-9, remained cohesive and did not rupture, even under increased deformation.

We demonstrate experimental MMP-9 effect on treated penis foreskin: increased deformation range due to reduced stiffness.

### Adhesive Capsulitis (Stiff Shoulder)

See FIG. 4 and Table 4. Pathology of adhesive capsulitis is evaluated by parameters (1) rotational range of motions (ROMs) and (2) glenohumeral (GH) stiffness which is decreased due to pathological stiffness of joint connective tissues (Lin, Hsu et al. 2008). The evaluation of adhesive capsulitis disease severity and efficiency of therapy is evaluated in mechanical and geometrical terms as stress-strain characteristics, ROM and GH stiffness, example references are (Lin, Hsu et al. 2008; Koh, Chung et al. 2012)

### Stiff Eye Lens

Reviewed in “Ophtalmology” section.

Safe Removal of Tissues

Sometimes there is a need for removal part of tissue from the body. It may be a healthy tissue (like transplant or biopsy) or unwanted tissue (tumor, wart, papilloma, scar, necrotic tissue). Mechanical excision provides a solution, however, we suggest that combination of mechanical approach with TREZ-softening of tissues may be beneficial.

The major needs are:
1) ability to examine the tissue prior to excision (surgeon makes a solution where to place the excision borders)
2) cause minimal damage to surrounding healthy tissues
3) in case of tumors: make sure that all tumor tissue is removed
We suggest that use of TREZ together with mechanical excision allows following benefits:
(1) Softened tissues can be pulled out and examined (without causing damage to functional integrity), so that surgeon can make desition where to cut
(2) Soft tissues could be more easily removed
(3) Less stress (tension) would be applied to neighboring tissues while removal
(4) Forming lesions would be easier to heal
(5) In some cases we observed that TREZ-treatment makes cells within tissues more cohesive and less prone to detachment: which is essential in case of removal of tumor (cancerous) tissues

### Biopsy or Transplant Material Derivation:

Retrieval of biopsy or transplant material is a valuable method in diagnostics and surgery, however, the method is invasive and has certain health risk. We suggest, that application of TREZ before tissue material retrieval or posterior treatment of lesion may improve ability of organ to heal the lesion.

### Tumor Surgery: Reduce Risk of Posterior Metastasis

Removal of tumor is an efficient method of cancer treatment, however, it carries a risk of accidental escape of cancerous cells and, therefore, metastasis. Methods that would allow to reduce risk of malignant cells “escape” during the surgical procedure would reduce risk of metastasis and would be highly desirable.

Certain tissues, for example, liver, have high risk of single cell detachment after lesion. In our experimental with liver tissues, we observed treatment of liver with MMP-9 improved cohesiveness between liver cells and surrounding extracellular matrix. Cells easily detached from non-treated or buffer-treated samples of liver tissues, however, cells were cohesive within MMP-9 treated liver tissue sample and risk of detachment was notably lower.

We suggest that mechanism of the effect can be explained by MMP-9 driven modification of liver extracellular matrix from highly crosslinked stiff material into cohesive gel. Viscous gels, compared to crosslinked stiff materials, have better cohesive and adhesive properties.

We suggest, that risk of accidental escape of malignant cells from surgically removed tumors may be reduced by making (1) tumor tissues and (2) adjacent healthy tissues more cohesive and gel-like by treatment by TREZ (for example, MMP-9).

### Cosmetic Removal of Scars, Warts, Papillomas, Etc.

Bacterial collagenases were used to digest tissues of scar in order to remove it. We suggest that TREZ may be used for removal of cosmetic defects, however, by a different mechanism.

We suggest that TREZ-softening is to be applied to the healthy tissues surrounding the abnormal tissue (example: healthy skin outside the wart, papilloma or scar) and surgical incision would be made within TREZ-treated healthy tissues. Thus, the whole abnormal tissue or structure can be removed completely and only healthy tissues would remain after such operation. Complete removal would be beneficial because warts and papillomas may re-grow if part of it remains after removal procedure.

### Debridement (Burn Wounds, Ulcers, Necrotic or Ischemic Tissues)

The method is reviewed in previous chapter.

### Hair Removal

There is a need for hair removal in cosmetics; however, the method should be safe for skin and not cause blistering or pain.

We suggest that TREZ-softening of skin or hair follicles would allow easy and painless mechanical removal of hair.

Solid Implants Insertion

There is a need to in medicine to insert a solid object within living tissue. Such could be: artificial implant, stunt, bone implant, reproduction control device, intrauterine device, devises for controlled release of drug (example: insulin release device), pacemaker. Insertion of a foreign body may cause local stress in adjacent tissues, resulting in shape distortions, cell damage or microscopic ruptures.

We suggest that TREZ-treatment of tissues where the foreign object to be inserted would allow to reduce traumatic effects (reduce shape distortion, cell damage, rupture or erosion). We suggest the following applications could benefit from TREZ-treatment (but not limited to examples named below):

stunt, bone implant, reproduction control device, intrauterine device, devises for controlled release of drug (example: insulin release device), pacemaker

Liquid or Gel-Like Implants Insertion

Insertion of gel-like or liquid implants (such as: collagen, hyaluronic acid, silicone) is highly used in cosmetic medicine and plastic surgery. It would be desirable (1) to allow uniform, smooth distribution of implant and avoid local bulging after injection of liquid or gel-like implant, (2) to fuse the implanted material (for example, natural extracellular matrix molecules of collagen or hyaluronic acid) with treated tissue. However, the natural stiffness of mammalian tissues hinders implant uniform distribution and merging.

We suggest that use of TREZ for softening of the target tissue would (1) make tissues more plastic, therefore, adapt better to injected gel and allow smooth distribution of it, (2) if tissue is treated in concentration sufficient to modify treated extracellular matrix from highly crosslinked into poorly crosslinked, viscous gel, it would allow the transplant molecules to merge and fuse with the extracellular matrix and even incorporate within it, if injected material is, for example, collagen or hyaluronic acid.

Dentistry

Dentistry has many needs, such as: to correct positioning of teeth or reconstructive medicine, that are limited by high stiffness and low plasticity of orthodontal tissues. It would be desirable to increase (temporarily) the plasticity of the orthodontal tissues, however, on condition of functional integrity (to avoid ruptures, blisters or teeth detachment the tooth socket). We suggest that TREZ-treatment would allow such therapies.

Correct tooth positioning: Improve effect of dental braces

### Enable Easier Teeth Eruption

Process of teeth eruption in infants is sometimes long and painful. It would be beneficial to make the process shorter and less painful by reducing stiffness of connective tissues around the erupting tooth, however, on condition that the tooth does not detach from the tooth socket. We suggest that TREZ can be used for such cohesive softening that would improve process of tooth eruption.

### Transplantation of Jaw Bones or Teeth

Can be aided by use of TREZ by method discussed in chapter devoted to use of TREZ in transplantation.

### Reshaping of Jaw Bones or Teeth

Can be aided by use of TREZ by method discussed in chapter devoted to bones shape modification by TREZ.

### Transplantation of Jaw Bones, Teeth or Other Organs Generated by Means of Regenerative Medicine

Discussed in chapter below.

### Transplantation of Stem Cells in Order to Repopulate Tissues

Softening of tissues in order to improve process of cells penetration is discussed in a special chapter.

Treating Fibrosis or Cirrhosis

### Liver Fibrosis and Liver Cirrhosis

Pathogenesis of liver fibrosis and liver cirrhosis has direct correlation with stress-strain characteristics of the liver tissue, such as increased stiffness. Level of stiffness increase has direct correlation with severity of the disease, as shown by (Wang, Changchien et al. 2009; Wang, Palmeri et al. 2009; Dechene, Sowa et al. 2010). Tissue stiffness interferes with regeneration and expansion of tissues. Stiffness of liver precedes extracellular matrix accumulation; therefore, stiffness (increased stress-strain ratio) is not a consequence, but part of pathogenesis in liver fibrosis and cirrhosis (Georges, Hui et al. 2007; Friedman 2010).

We demonstrate experimental quantitative effect of MMP-9 on treated liver tissues: stiffness being reduced by application of MMP-9 (see FIGS. 19, 20).

Increased stiffness (increased stress-strain ration) of other permanent tissues has impact on pathology and progression of different types of fibrosis, such as: pulmonary fibrosis, endomyocardial fibrosis, mediastinal fibrosis, myelofibrosis, retroperitoneal fibrosis, progressive massive lung fibrosis, kidney fibrosis, intestinal fibrosis in Crohn's disease, arthrofibrosis, capsulitis.

Myocardial fibrosis pathology correlated with increased stress-strain ratio (Carroll, Janicki et al. 1989). Increased stiffness of fibrotic myocardium reduces function of myocard and increases risks of heart failure.

Regenerative Medicine

Regenerative medicine offers opportunities of replacing a degenerated organ with a functional one, artificially engineered, or expanded from healthy tissue, or generated from human cells, untreated or modified, differentiated or stem cells in vitro. It would be highly desirable to expand small parts of healthy tissue, that can be taken as biopsy, into large ones for transplantation purposes. Possibility to expand in vitro whole tissues or organ parts instead of single cells could potentially allow to expand the functional organ of organized complexity (for example: podocyte cell culture expanded in vitro would not possess the functional organization of whole kidney). Therefore, methods to expand whole tissues would be highly desirable. However, the tissue proliferation ability is much lower compared to proliferation to single cell cultures comprising same tissue. Apparently, stiffness of tissue and low plasticity of it set strict limitation on possibility of tissue to expand (that is how mammalian organisms restrain size of internal organs).

We suggest that ability of TREZ-treated tissues to reduce stiffness, increase plasticity and permeability would allow to improve techniques of regenerative medicine:
1) expand tissues by overcoming limitations on tissue expansive growth, set by natural stiffness of extracellular matrix
2) allow better permeability of tissues, therefore, improving possibility to grow tissues in vitro
3) allow better penetration of cells into decellularized matrices, used for purposes of regenerative medicine (reviewed further)
4) improve general methods of surgery, transplantology, reconstructive surgery that are part of regenerative medicine technique

Cosmetics

TREZ can be applied to mammalian (human) tissues not only by surgical way, but also as part of cosmetics compositions, such as application crèmes, gels, lotions, serums etc.

There are numerous applications in cosmetics that could benefit from possibility to reduce stiffness of permanent tissues (such as skin), for example:

### Reduce Wrinkles and Smoothen Skin

As it was discussed in previous chapter, wrinkles are difficult to remove by purely mechanical mean due to natural stiffness and low plasticity of adult skin. Possibility to use aggressive enzymes that actively dissolve or digest tissues is limited because of risk of blistering and ulceration.

We suggest that use of TREZ as part of a cosmetic composition would make it more plastic in order to reduce extent of wrinkling. We experimentally demonstrate that TREZ, when applied to skin, can improve it's plasticity and maintain cohesiveness and functional integrity of skin (see FIG. 17).

### Reduce Risk of Scarring

Proteolytical enzymes are traditionally used to treat scars caused by (1) acne, (2) dermatological pathologies, (3) fragility or sensitivity of skin. The enzymes are traditionally used to be applied to scars and dissolve them.

We suggest that use of TREZ can serve the same need, but in a different method. The TREZ can be applied to skin suffering fragility, rupture, erosion, lesion (caused by acne or other pathology) in order to (1) reduce risk of rupture and erosion, (2) reduce risk of scarring by the mechanisms we discussed in previous sections.

Culturing Tissue Explants In Vitro

Including such applications as (but not limited to those named below):
for following transplantation
to study culture in vitro
to culture and passage tissue in vitro while maintaining it's functional integrity

The use of TREZ for need to culture organotypic, artificial or other tissues or 3D structures like pancreatic islets in vitro was discussed in detailed in previous chapters, devoted to techniques of islet in vitro culture and regenerative medicine.

Tissue Explants In Vitro Used for Drug Screening

There is a need in pharmacology to perform in vitro screening of drug candidates on living systems (such as cells or tissues) prior to in vivo studies, in order to exclude the candidates with obviously strong side effects or obviously inefficient candidates.

Drug screening techniques are well developed for single cell cultures, such as hepatocytes, vascular endothelial cells or stem cells. However, tissue cultures would be more beneficial for in vitro drug screening, as cells within tissues are more close imitation of in vivo cells. Importantly, cells maintain their natural niches when cultured in vitro within cells, therefore, maintain original phenotypes and behave more close to how they would behave in live system.

It would be highly beneficial to adapt tissue cultures for routine, automated large-scale techniques of drug screening, that strongly involves robotic techniques nowadays. We discussed how TREZ-treatment of in vitro tissue cultures would improve technique of their maintenance. In this section we shall particularly describe a need to microscopically observe and quantify the drug affected behavior of in vitro tissue cultures, in methods robust enough to be compatible with robotic systems.

Main problem of 3D tissue samples is large thickness, which sets complications for automated microscopy and evaluation techniques. We suggest that TREZ can be used to reduce thickness and to adapt plastic tissue samples into desired shapes, compatible with automated microscopy techniques, however, maintaining cohesiveness and functional organization of tissue samples. On example of pancreatic islets we show, that MMP-9 treatment allows to spread them into very thin, yet cohesive layers, that would be perfectly compatible with automated microscopy techniques (see FIG. 23). Untreated islets, as we show, at attempt to spread them to same extent, undergo rupture: the cells separate from each other.

Veterinary

Same methods that were discussed for human medicinal use, may be applied to treatment of animals.

Non-Living Tissues: Modifying Physical Properties

TREZ-treatment of tissues can also be applied to non-living tissues. Here we review several applications (though, not limited to those).

### Decellularized Tissues and Organs for Transplantation Purposes

Use of decellularized tissues and organs for transplantation purposes is a cost-efficient and highly beneficial method in transplantation and regenerative medicine. Extracellular matrix from allogenic or xenogenic donor can be transplanted and be inhabited by own cells of the patient or histocompatible donor (variants: stem cells, stem cells expanded in vitro, stem cells differentiated in vitro, adult cells, iPS cells, etc).

Advantages of transplanting the extracellular matrix (decellularized tissue or organ) from allogenic or xenogenic donor are:
1) Extracellular matrix, devoid of cells, will have no histocompatibility problems and immune rejection risk when transplanting from human to a human
2) Extracellular matrix is highly conservative between species. Therefore, transplantation of only extracellular matrix from animal donor (for example, pig) to human patient would have lower risks for rejection.
3) Extracellular matrix, decellularized, can be sterilized (for example: by irradiation, by autoclaving) in order to reduce risk of allogenic or xenogenic pathogens (for example: bacteria, viruses)
4) Extracellular matrix, decellularized, can be stored for long time (months, years), cold or frozen (+4 C, −20 C, −80 C)

Decellularized tissues and organs that are developed, include:
1) Decellularized blood vessels
2) Decellularized liver
3) Decellularized kidney
4) Decellularized bone
5) Decellularized skin
6) Decellularized lung
7)

Reasons to use extracellular matrix of whole organ instead of soluble molecules mix:
1) Extracellular matrix of whole organ has nature-wise compartmentalization, architectural organization, which is beneficial for organ maintenance. When extracellular matrix is minced or solubilized, the complex structure is lost. The geometrical parameters are not the same, if soluble molecule mixture is delivered as gel or powder.
2) Organ stiffness: cell niche and differentiation. Native extracellular matrix has stiffness that defines cell behavior. For example, stiffness of extracellular matrix can define differentiation patterns of stem cells (Engler, Sweeney et al. 2007)
3) Organ stiffness: organ durability. Original bone extracellular matrix has mechanical properties (stiffness, stress-strain parameters, tension-wise and compression-wise durability) that are lost when demineralized extracellular matrix is introduced as powder

TREZ-treatment can be useful for extracellular matrix devoid of living cells. Whole extracellular matrix, devoid of cells, has same limitations for transplantation as a living transplant does. Treatment with TREZ prior to transplantation can improve following parameters:
1. Ability to stretch and adjust geometrical parameters (thickness, coverage area, complex shape)
2. Ability to adjust to complex shape of the defect (bode defect, skin wound, cartilage crack)
3. Ability to fit the complex shape without gaps, that would enhance:
   a. Cell permeation
   b. Vascularization (if needed)
   c. Exchange of gases, nutrients and waste products
4. Reduce risk of transplant detachment

### Embalming

Embalming techniques may require plastic modification of soft tissues, modification of shape and expression. Side effects caused by loss of functional integrity, such as blistering and ulceration, would be unwanted.

Use of TREZ for plastic modification of tissues can be beneficial for embalming techniques. As TREZ are natural enzymes, use of those would be ecologically compatible.

### Food Industry

Proteolytic enzymes and especially collagenases are used in food industry, for example, for meat processing in order to make meat softer. Collagenolytic enzymes have advantage compared to non-collagenolytic, because collagen scaffold of meat extracellular matrix is what makes it stiff.

TREZ can be used for same purpose, however, use of TREZ has certain advantages compared to aggressive (bacterial, digestive) collagenases. Meat softening would require (1) reducing stress-to-strain ratio (in order to apply less effort to chew meat), however (2) meat should maintain elasticity and architecture (treated meat is marketed as meat, not minced meat). Treatment of meat with TREZ results in softening, however, maintaining architecture of meat (it does not blister, fall apart or dissolve into ulcers).

As TREZ are natural enzymes, they have advantage of being ecological (compared to chemical substances).

### Fur (Hyde) Industry

In fur industry there is are several needs, that can be solved by use of TREZ:

Need 1: Reduce weight of fur or hyde, however, maintaining it's integrity. People want to wear light clothes, shoes or bags, however, the products should not fall apart or suffer rupture. It is essential for fur industry, that the hair retains connection with skin. This can be achieved by softening dermis layer by TREZ and removal by mechanical techniques.

Need 2: Modify shape of fur or hyde (in order to achieve a complex shape or curvature for objects of complex design).

As TREZ are natural enzymes, they have advantage of being ecological (compared to chemical substances).

### I

### EXPERIMENTAL

### Materials and Methods

### Tissue-Remodeling Enzymes

We have used human recombinant tissue-remodeling enzymes of matrix metalloproteinase (MMP) family and cathepsin family. To achieve properties close to those of native enzymes, we used (1) full-size enzymes in order to have all domains, (2) produced in mammalian (human or Chinese hamster) cells in order to attain proper glycosylation, (3) in lyophilized form in order to achieve enzyme high concentration, up to 1 mg/ml.

Activation was performed according to standard protocols: 4-aminophenylmercuric acetate (APMA) activation (0.5-1 mM) at neutral pH (pH7.5-8.0) in presence of calcium ions (1-10 mM) for enzymes of MMP family and disulfide bonds reduction by 5 mM DTT (dithiothreitol) at acid pH (pH 5.0) for the cathepsins, followed by removal of activator. We would generally recommend to follow producer's instructions on enzyme reconstitution, activation, and storage.

Zymogens (full-size, pre-activated forms of enzymes) were restored from lyophilized form (according to producer's protocols) and transferred into activation buffers by means of dialysis. Buffer replacement (for example, to remove the activator) was performed by use of dialysis chambers with membrane MWCO (molecular weight cut-off) as 10,000 Da. Method of dialysis allows to change the buffer composition and maintain high concentration

Detailed procedures for MMP activation are reviewed in the book “Matrix Metalloproteinase Protocols” by Clark, Ian M, published 2001, Methods in Molecular Biology, ISBN 1592590462, Vol. 151 and numerous academic publications devoted to MMP studies.

### Tissue Samples

Murine organs were placed in Hank's Balanced Salt Solution (HBSS) under dissection microscope, and uniform sections of tissue were excised by methods of microsurgery. Each section was dissected further into several uniform segments, that would serve as for experimental and for control group. As each animal has individual stiffness properties of organs (dependent on age and lifestyle), we aimed that tissue samples for experiment and control have to be derived from proximal tissues of same animal to attain maximal uniformity.

For reshaping and gel properties assays we generated the samples of tissues with well-defined angles of parallelepiped-like shape. In later assay the stiff tissues that behave like solid bodies would maintain the shape and angles, while gel-like tissues change to spherical shape with smoothened or undistinguishable angles.

Tissue samples were preliminary examined and photographed, prior to enzyme application.

Tissue types, used for our studies, were: skin, tendon (major limb tendons, including Achilles and minor finger tendons), cartilage (of nose and of ear), subcutaneous soft tissues of lip, penis foreskin, subcutaneous soft tissues of penis, liver, soft tissues of uterus wall, ovaries, pancreas, pancreatic insulin-producing islets of Langerhans, eye lens, nerve bundle (sciatic nerve, optic nerve), muscle, musculotendinous junction.

### Application of Enzymes to Tissue Samples:

For in vitro assays: uniform tissue samples were generated, photographed and placed into plastic tubes, activated enzyme or control buffer solution being placed above. Tissue samples were gently rinsed by applied solution before incubation. Tissue samples were incubated at 37 C at water bath (for non-living tissues) or cell culture incubator (for living tissues), time of incubation varying for each experiment (from 30 minutes to 4 days). During incubation tissue samples were gently rinsed by applied solution about 1-2 times per day. After treatment the samples were placed into HBSS solution devoid of enzymatic activity and subject to set of assays to qualify functional integrity maintenance, mechanoelastic and viscoelastic properties (such as: stiffness, plasticity, adhesivity, poroelasticity, fragility, cohesiveness, etc).

In vivo assay: thin segments of skin including dermis were generated in flap-like technique on anesthesized animals and activated enzyme vs control buffer were applied in form of solution were applied to each flap-like tissue segment in situ. The segments were rinsed by fresh solutions (of activated enzyme or buffer) several times per day for 2 days and solutions, after rinse, were kept applied to the treated segments. After treatment, the enzyme- and buffer-treated skin segments were excised and subject to set of assays to qualify functional integrity maintenance, mechanoelastic and viscoelastic properties (such as: stiffness, plasticity, adhesivity, poroelasticity, fragility, cohesiveness, etc).

### Functional Integrity: Qualitative Evaluation

Each tissue sample was examined for maintenance of functional integrity after enzyme vs buffer treatment. Certain criteria of functional integrity are tissue-specific, such as: exocrine duct system integrity in pancreas, cohesion between muscle fibers in muscle, cohesiveness between two proximal ends of tendon. Tissue-specific criteria for functional integrity will be named for each tissue individually. However, general criteria are:
1. Does the tissue sample remain as one whole entity? Example: MMP-9 treated tendon stays as one whole entity, cohesiveness existing between two proximal ends. Clostridial collagenase-treated tendon is digested into microscopic floating particles, non-cohesive between each other.
2. Does the tissue maintain cohesion between neighboring cells?
3. Does the tissue undergo ruptures?

Functional integrity was observed (1) for tissue sample floating in buffer, (2) tissue sample being fixed and spread by means of a coverglass, (3) for tissue samples being subject to increased pressure loads.

We used different ranges of microscope magnification, from lowest to ×40 in order to monitor macroscopic and microscopic (such as cell cohesion and microscopic ruptures) effects. We used several types of microscopy: (1) phase contrast on inverted microscope, (2) brightfield mode on dissection microscope, (3) dark field microscopy on dissection microscope. In phase contrast mode we variated settings for phase in order to distinguish between objects with similar transparity.

### Stress-Strain Characteristics for Quantitative Assay

Stress-strain characteristics of tissue are used in several assays and applications, such as: stiffness, creep assay, deformation under permanent load, residual deformation after temporary load.

Methods we used are a straightforward methods used for material stiffness evaluation in mechanics. However, we adjusted the mechanics methods to small size of our tissue specimen:

### Method 1: Apply Stress to the Tissue Specimen and Measure Relative Deformation.

The tissue specimen are placed in 24-well or 6-well tissue culture plate on coverglass and covered with HBSS buffer. The plate with specimen were placed under dissection microscope and photographed. Loads of progressive mass were applied to the specimen covered with coverglass; shape of load being thoroid in order to allow photograph the sample under the load. Coverglass placed upon the specimen were immersed in same buffer in order to avoid capillary effects that would distort the stress evaluation. The specimen were subject to increading loads of progressive mass. The specimen was photographed before and after the loads were applied; the surface area was measured by imaging program (in pixels, number being proportional to surface area).

Stress was evaluated as force (calculated as gravity force equal to load mass multiplied by g (strength of the gravitational field) and multiplied by calibration index to evaluate buoyant forced (caused by load being immersed in buffer)) being divided by the specimen area (to which the force is applied).

Strain was calculated as relative deformation of sample. Relative area is calculated difference in areas after load and prior to load, calibrated to initial area of specimen. Relative thickness of sample was estimated as reverse proportion of relative area of the specimen.

### Method 2: Apply Deformation to the Tissue Specimen and Monitor the Resulting Stress Exhibited by Specimen.

The tissue specimen were placed sensitive scale. A depth micrometer (Starrett 445MBZ-150RL Vernier Depth Gauge) is rigged on a stand above the sample. Micrometer screw applied a defined permanent deformation to a tissue sample. The resulting stress is measured on the scale readout and the change of stress in time was observed.

### Methods to Assess Plasticity

Plasticity Characteristics (Residual Strains after Temporary Stress Applied)

The assay measures relative residual deformation (strain) after temporary stress applied. Permanent stress was applied for 2 minutes, afterwards removed and the specimen was allowed to restore previous shape for another 2 minutes. Residual deformation was measured by technique described above.

### Qualitative Assays:

See qualitative assays for reshaping

### Methods to Assess Stiffness

### Stress-Strain Characteristics (Stiffness)

The assay measures relative deformation (strain) permanent stress applied as described before. Permanent load was applied to a tissue sample placed under dissection microscope, properties of load adjusted to allow photographing of the tissue sample under the load. Photographs were taken of tissue sample before loads, and following a series of increased loads. Stress and strain valued were calculated as described above.

### Methods to Assess Elastic Vs Viscous Reaction to External Stress/Deformation

### Qualitative: Gel or Elastic Material

Tissue samples, treated by enzymes or buffer, can be qualitatively evaluated: do they behave like elastic material (like they used to before treatment) or like gels.

### Creep Assay

We estimated viscoelastic properties of treated vs untreated tissues by applying permanent strain (as described above) and monitoring the kinetics of resulting stress within tissue sample. We use qualitative scale to represent the results of creep assay. MMP-treated tissues showed larger creep than untreated.

### Methods to Assess Adhesivity

We used a series of qualitative assays to assess adhesivity of tissue samples that were disclosed and discuss in previous chapters.

### Methods to Assess Cohesiveness

We used method of high-magnification microscopy to observe, whether individual cells within the treated tissues remain cohesive. If sample is moved, shaken or stretched under the microscope, we observe if individual cells in view field behave in cohesive manner and remain attached to the neighboring cells.

### Ability to Attain Different Shape (Reshaping)

We used a series of simple qualitative assays (also explained and discussed in previous chapters) to assess ability of the tissue sample to attain a different shape, for example:
1) ability of sample to attain a round shape within inoculation loop
2) ability of sample to attain conical shape, when placed within plastic pipette tip and subject to mild centrifugation, followed by relaxation period of 10 minutes
3) ability of sample to attain a different shape, when placed upon coverglass and moved by needles (stiff tissues move, but retain shape, while reshapable tissues easily attain different shapes).

Our qualitative observations correlate with quantitative results from plasticity assay.

### Permeability

Permeability depends on thickness of individual elements within tissue sample. Effects of increased permeability of reduced thickness can be evaluated for MMP-9 treated tendon and MMP-9 treated pancreas; as seen on FIGS. 7-11.

### Experimental Results

### Screening Different Enzymes and Tissues

Preparations of human recombinant proteinases MMP-1, MMP-8, MMP-9, Cathepsin B and Cathepsin S (Sino Biologicals) were activated and transferred into working buffer of appropriate pH (neutral for MMP's and pH5.0 for cathepsins) and ionic additives (calcium ions for MMP's in concentration from 1 to 10 mM). The enzymes were diluted to concentrations of 0.03 mg/ml and applied to lip connective tissue samples and several other tissue types. Collagenase of Clostridium histolyticum (Roche) and crab collagenase (YO Proteins) served as controls for aggressive collagenolysis. Buffers used for dilution of MMP's and Cathepsins served as negative controls. The samples were observed for several days and evaluated by qualitative assays to (1) functional integrity maintenance, (2) stiffness reduction, (3) plasticity enhancement, (4) viscous reaction to external stress/deformation, (5) ability for reshaping.

According to assay, of MMP's and cathepsins group we selected a number of most representative experiments, that were reproduced later in different concentrations and time exposures and evaluated quantitatively in terms of stress-strain characteristics. Majority of experiments were performed with MMP-9, as it was, in our assay, most efficient to reduce stiffness and increase plasticity while maintaining functional integrity in vast range of tissues, from stiff constructive tissues (like tendon) to soft tissues (like liver or pancreatic islets). Vast majority of experiments was performed in vitro in order to reduce risk of accidental deformation of treated tissue samples, that may accidentally happen when applied to live animals in situ. Mouse tissues were used, unless otherwise specified.

Lip Connective Tissue: Reduced Stiffness and Increased Plasticity after MMP-9 Treatment

Subcutaneous tissues of lip contain highly innervated muscular and connective tissues. They are highly sensitive, responsible for mimics and mouth function. There are medical and cosmetical needs for modification of mechanic and shape characteristics of lip: (1) in reconstructive surgery (such as: cleft lip, lip tissues reconstruction after tumor removal), (2) in plastic surgery: to modify shape or size of lip, (3) in transplantology, (4) in cosmetic surgery, such as, insertion of implant materials (such as: collagen, silicone) into lip tissue in order to increase volume. Functional integrity requirement for such procedure would be (1) to maintain lip tissues cohesive (to avoid blisters and ulcers), (2) to avoid nerve rupture (such would cause pain, numbness, loss of motility)

Assay:

Uniform samples of subcutaneous lip tissues were isolated from mouse lip after skin removal by methods of microsurgery and were incubated for 4 days at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed by applied solution every day.

To assay compatibility with nerve integrity, uniform samples of sciatic nerve bundle were isolated and were treated by same enzymes formulation at same conditions.

Results:

Tissue samples treated by MMP-9 (1) decreased stiffness, (2) increased plasticity, (3) improved adhesiveness while maintaining functional integrity. Tissue samples treated by collagenase of Clostridium histolyticum were dissociated into non-cohesive floating microscopic particles. Tissue samples treated by buffer (1) retained original stiffness and ability to maintain shape and (2) remained cohesive.

Table 5 shows summary of qualitative assays.

FIG. 13 shows quantitative evaluation of stress-strain characteristics of lip tissue samples such as stiffness and plasticity, ability to increase area and to decrease thickness under stress.

FIG. 14 demonstrates effect of increased plascitity of MMP-9 treated lip tissue, compared to buffer-treated control.

FIG. 15 demonstrates effect of MMP-9 rendering lip tissue gel-like (adhesive, permeable, reshapable), compared to buffer-treated control.

Lip Connective Tissue: Reduced Stiffness and Increased Plasticity after Cathepsin B Treatment

Subcutaneous tissues of lip was treated by human recombinant Cathepsin B (concentration of 0.03 mg/ml; activated by 5 mM DTT at pH5.0 for 15 minutes at room temperature, which was subsequently removed by dialysis) vs buffer. The lip tissue was incubated with Cathepsin B vs buffer for 20 hours at 37 C, being gently rinsed several times. Cathepsin B treated samples were of reduced stiffness and of increased plasticity compared to buffer-treated samples.

FIG. 16 shows quantitative evaluation of stress-strain characteristics of lip tissue samples such as stiffness and plasticity, ability to increase area and to decrease thickness under stress.

Lip Connective Tissue: Reduced Stiffness and Increased Plasticity after MMP-8 Treatment

Subcutaneous tissues of lip was treated by human recombinant MMP-8 (concentration of 0.03 mg/ml; activated by AMPA 0.5 mM at pH8.0 at calcium ions concentration of 10 mM for 2 hours at 37 C) vs buffer. The lip tissue was incubated with MMP-8 vs buffer for three days at 37 C, being gently rinsed 2 times per day. Qualitative evaluation shows that MMP-8 treated samples were less stiff and of increased plasticity compared to buffer-treated samples.

Skin Dermis: Reduced Stiffness and Increased Plasticity after MMP-9 Treatment

Skin is a complex organ and it's proper function depends on cohesiveness within comprising tissue elements and between them (such as illustrated on FIG. 2 and Table 3).

There are medical and cosmetical needs for modification of mechanic and shape characteristics of skin, such as: (1) skin transplantation (to increase area, reduce thickness and adjust to shape of wound or lesion, (2) in reconstructive surgery, (3) in surgery: to reduce risks of scarring by making skin tissue more adhesive and plastic. Functional integrity requirement for such procedure would be (1) to maintain cohesiveness within dermis and within epidermis (to avoid ruptures and wounds), (2) maintain integrity of dermal-epidermal junction, (3) maintain coherence between hair and skin, to avoid alopecia, (4) to avoid nerve rupture (such would cause pain and numbness)

Assay:

Uniform samples of whole skin (including dermis, epidermis and hair) were isolated by methods of microsurgery and were incubated for 2 days at 37° C. in solution of: (1) MMP-9, 1 mg/ml, preactivated with 0.5 mM APMA, (2) collagenase of Clostridium histolyticum, 1 mg/ml, (3) buffer solution. Samples were gently rinsed by applied solution every day and were subject to qualitative assay of integrity and mechanical characteristics.

Uniform samples of skin dermis (isolated from epidermis) were isolated by methods of microsurgery and were incubated for 2 days at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed by applied solution every day and were subject to quantitative stress-strain and geometrical modification assessment, and for qualitative evaluation.

To assay compatibility with nerve integrity, uniform samples of sciatic nerve bundle were isolated and were treated by same enzymes formulation at same conditions.

Results:

Tissue samples treated by MMP-9 (1) decreased stiffness, (2) increased plasticity, (3) improved adhesiveness while maintaining functional integrity. Tissue samples treated by collagenase of Clostridium histolyticum were dissociated whole tissue into non-cohesive floating microscopic particles (hair remained intact, but detached). Tissue samples treated by buffer (1) retained original stiffness and ability to maintain shape and (2) remained cohesive.

Table 5 shows summary of qualitative assays.

FIG. 17 shows quantitative evaluation of stress-strain characteristics skin dermis samples such as stiffness and plasticity, ability to increase area and to decrease thickness under stress.

Tendons: Cohesive Architecture Remodeling, Reduced Stiffness and Increased Plasticity after MMP-9 Treatment

Tendons are highly durable and highly elastic tissues. Each tendon consists of interconnected fibrils, tightly packed in order to ensure high durability. There are medical and cosmetical needs for modification of mechanic and shape characteristics of tendon: (1) to decrease stiffness in aged or traumatized tendon, (2) to enable tendon expansion (in case of short tendon), (3) to expand tendon tissue in vitro for medical and veterinary purposes. Functional integrity requirement for such tendon modifications would be: (1) to maintain connection between individual fibrils comprising tendon, so that network of interconnected fibrils behaves as whole, (2) to avoid local ruptures

Assay 1:

Uniform samples of large limb tendon (Achilles or other) were isolated from mouse hind limbs by methods of microsurgery and were incubated for 1 day at 37° C. in solution of: (1) MMP-9, 1 mg/ml, preactivated with 0.5 mM APMA, (2) collagenase of Clostridium histolyticum, 1 mg/ml, (3) buffer solution. Samples were gently rinsed by applied solution.

Assay 2:

Uniform samples of large limb tendon (Achilles or other) were isolated from mouse hind limbs by methods of microsurgery and were incubated for 3 days at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed by applied solution, daily.

Assay 3:

Uniform samples of finger tendon were isolated from mouse hind limbs by methods of microsurgery and were incubated for 1 day at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed by applied solution.

Results:

Tendons, treated by MMP-9 (1) split into interconnected, cohesive network of individual fibrils, (2) increased plasticity, (3) improved adhesiveness, (4) improved permeability while maintaining functional integrity. Tissue samples treated by collagenase of Clostridium histolyticum were completely dissociated into non-cohesive floating microscopic particles (assay 1) or partially dissociated into non-cohesive microscopic particles, while remains of tendon were partially ruptured. Tissue samples treated by buffer (1) retained original stiffness and ability to maintain shape and (2) remained cohesive.

Table 5 shows summary of qualitative assays.

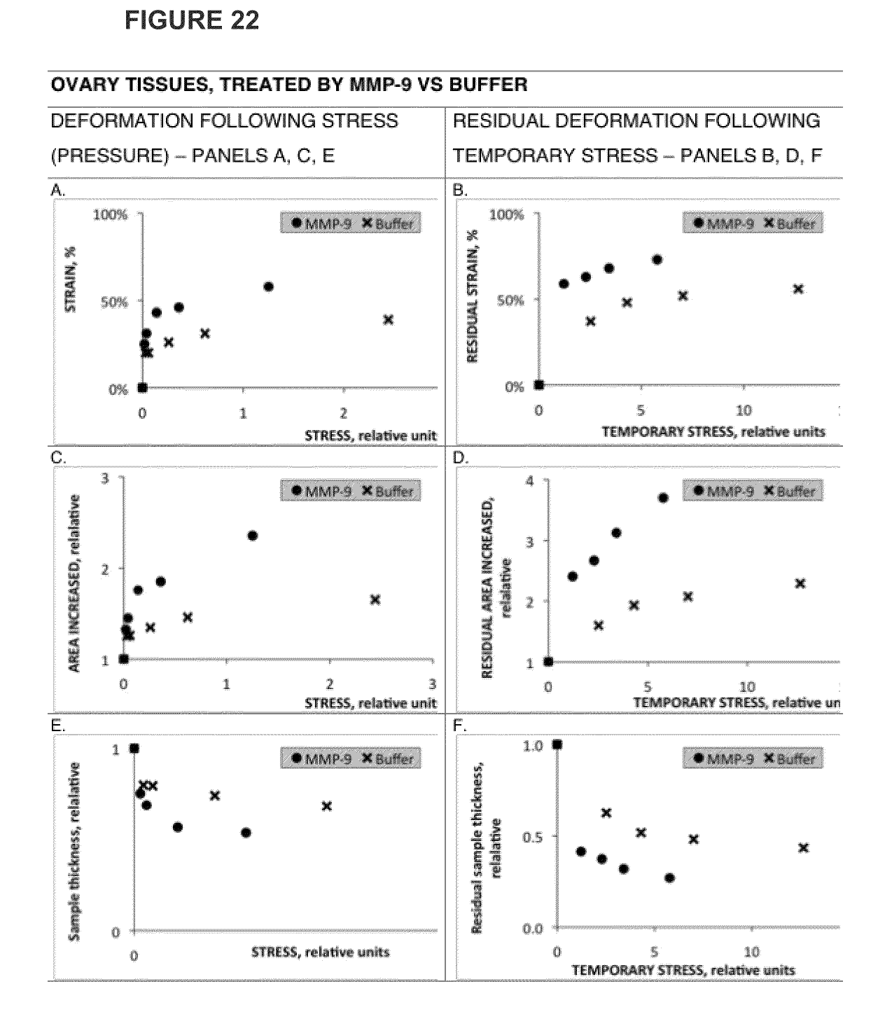

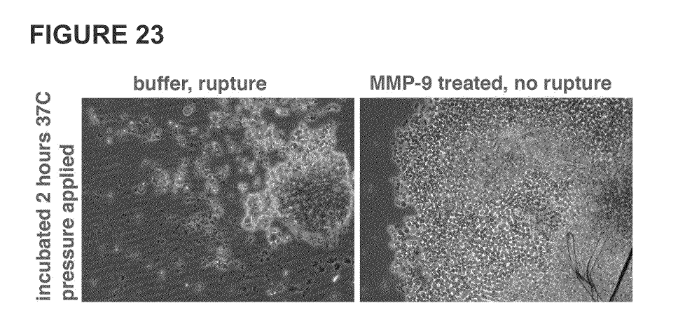

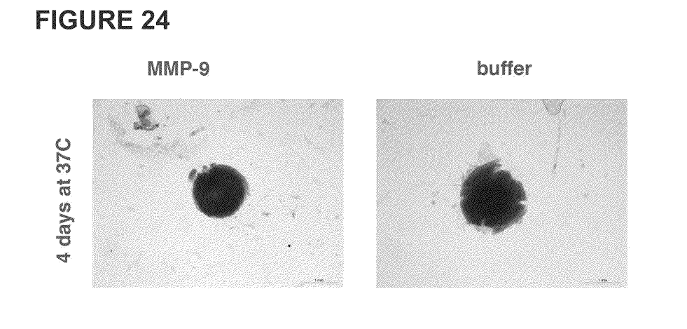

FIGS. 8, 9 shows difference in tendon dissociation pattern, when treated by MMP-9 (cohesive remodeling) vs collagenase of Clostridium histolyticum (non-cohesive digestion). FIG. 7 illustrates the effect as a drawing.

Cartilage (Mouse and Porcine): Increased Plasticity and Reduced Stiffness after MMP-9 Treatment

Cartilage is a flexible connective tissue comprising joints between bones, rib cage, ear, nose, bronchial tubes and intervertebral discs. Modification of cartilage mechanical and shape propertied would be desirable (1) in plastic and reconstructive surgery (example: rhinoplasty), (2) for treatment of herniated intervertebrate discs, however, on condition that treated cartilage does not degenerate, rupture or blister.

Assay:

Uniform samples of (1) nose cartilage (mouse) and (2) ear cartilage (mouse and porcine) were isolated as described above and incubated for 4 days at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed by applied solution every day.

Results:

Tissue samples treated by MMP-9 (1) decreased stiffness, (2) increased plasticity, (3) improved adhesiveness while maintaining functional integrity. Tissue samples treated by collagenase of Clostridium histolyticum were dissociated into non-cohesive floating microscopic particles. Tissue samples treated by buffer (1) retained original stiffness and ability to maintain shape and (2) remained cohesive.

Table 5 shows summary of qualitative assays.

FIG. 18 shows quantitative evaluation of stress-strain characteristics of samples such as stiffness and plasticity, ability to increase area and to decrease thickness under stress.

Penis: Foreskin and Subcutaneous Connective Tissues: Reduced Stiffness and Increased Plasticity after MMP-9 Treatment

Modification of mechanoelastic, plastic and geometrical properties of penis tissues would be desirable in treatment of such conditions as: phimosis, Peyronie's disease, reconstructive, plastic and cosmetic surgery, however, on condition (1) that cohesion of penis tissues maintains and (2) innervation is not ruptured by the method.

Assay:

Uniform samples of (1) penis foreskin and (2) subcutaneous connective tissues of penis were isolated by methods of microsurgery and incubated for 2 days at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed by applied solution every day.

To assay compatibility with nerve integrity, uniform samples of optic nerve were isolated and were treated by same enzymes formulation at same conditions.

Results:

Tissue samples treated by MMP-9 (1) decreased stiffness, (2) increased plasticity, (3) improved adhesiveness while remaining cohesive.

Tissue samples treated by collagenase of Clostridium histolyticum were dissociated into non-cohesive floating microscopic particles. Tissue samples treated by buffer (1) retained original stiffness and shape and (2) remained cohesive.

Table 5 shows summary of qualitative assays.

Liver: Reduced Stiffness and Increased Plasticity after MMP-9 Treatment

Modification of liver tissue stiffness would be desirable for treatment of fibrosis and cirrhosis; also for expanding liver tissues for transplantation purposes, however, on condition that the treatment would maintain the complex composition of liver cells and compartments cohesive.

Assay:

Uniform liver tissue samples incubated for 3 days at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed by applied solution every day.

To assay compatibility with nerve integrity, uniform samples of optic nerve were isolated and were treated by same enzymes formulation at same conditions.

Results:

Tissue samples treated by MMP-9 (1) decreased stiffness, (2) increased plasticity, (3) improved adhesiveness while maintaining functional integrity. Tissue samples treated by collagenase of Clostridium histolyticum were dissociated into non-cohesive floating microscopic particles. Tissue samples treated by buffer (1) retained original stiffness and ability to maintain shape and (2) remained cohesive. MMP-9 treated tissue allowed to maintain cells more cohesive within liver tissue sample, compared to buffer-treated tissue.

Table 5 shows summary of qualitative assays.

FIG. 19 shows quantitative evaluation of stress-strain characteristics of samples such as stiffness and plasticity, ability to increase area and to decrease thickness under stress.

FIG. 20 illustrates the effect

Uterus: Reduced Stiffness and Increased Plasticity after MMP-9 Treatment

Modification of uterine tissue stiffness, plasticity and adhesiveness would be desirable for infertility, to aid embryo implantation and placenta formation; however, on condition that the treatment would maintain the uterine tissues cohesive.

Assay:

Uniform samples of uterine tissues were isolated as described above and incubated for 4 days at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed by applied solution daily.

Results:

Tissue samples treated by MMP-9 (1) decreased stiffness, (2) increased plasticity, (3) improved adhesiveness while maintaining functional integrity. Tissue samples treated by collagenase of Clostridium histolyticum were mostly dissociated into non-cohesive floating microscopic particles. Tissue samples treated by buffer (1) retained original stiffness and ability to maintain shape and (2) remained cohesive.

Table 5 shows summary of qualitative assays.

FIG. 21 shows quantitative evaluation of stress-strain characteristics of samples such as stiffness and plasticity, ability to increase area and to decrease thickness under stress.

Ovaries: Reduced Stiffness and Increased Plasticity after MMP-9 Treatment

Modification of ovary stiffness and plasticity would be desirable (1) to treat infertility, (2) to improve methods of in vitro fertilization; however, on condition that the treatment would maintain the complex tissues of ovaries cohesive and non-ruptured.

Assay:

Uniform samples of ovaries tissue were isolated as described above and incubated for 4 days at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed by applied solution every day.

Results:

Tissue samples treated by MMP-9 (1) decreased stiffness, (2) increased plasticity, while maintaining complete cohesiveness and functional integrity. Tissue samples treated by collagenase of Clostridium histolyticum were dissociated into non-cohesive floating microscopic particles. Tissue samples treated by buffer (1) retained original stiffness and shape and (2) remained cohesive.

Table 5 shows summary of qualitative assays.

FIG. 22 shows quantitative evaluation of stress-strain characteristics of samples such as stiffness and plasticity, ability to increase area and to decrease thickness under stress.

Pancreas: adhesive architecture remodeling improving permeability after MMP-9 treatment

Improving permeability of pancreatic tissue would be desirable for diabetes treatmens: (1) pancreas transplantation and (2) in vitro expansion of pancreatic tissue, however, on condition that tissue maintains functional integrity, which includes cohesion between exocrine cells and integrity of lumens and intralobular ducts, necessary for exocrine function.

Assay:

Uniform samples of pancreas, approximately 1 mm in diameterm, were isolated from pancreas by methods of microsurgery and incubated for 1 day at 37° C. in solution of: (1) MMP-9, 1 mg/ml, preactivated with 0.5 mM APMA, (2) collagenase of Clostridium histolyticum, 1 mg/ml, (3) buffer solution. Samples were gently rinsed by applied solution.

Results:

Tissue samples treated by MMP-9 have changed the architecture: solid piece of tissue separated into interconnected lobules in manner that maintained cohesion of cells within lobules and integrity of intralobular ducts. The lobules thickness was substantially lower compared to that of original tissue sample, thus, remodeling improved tissue permeability. Tissue samples treated by collagenase of Clostridium histolyticum were digested into non-cohesive floating microscopic particles. Tissue samples treated by buffer (1) retained original stiffness and shape and (2) remained as one solid part.

Table 5 shows summary of qualitative assays.

FIG. 11 shows difference in pancrea dissociation pattern, when treated by MMP-9 (cohesive remodeling) vs collagenase of Clostridium histolyticum (non-cohesive digestion). FIG. 10 illustrates the effect as a drawing.

Pancreatic Insulin-Producing Islets of Langerhans: Reduced Stress-Strain Ratio and Increased Adhesivity after MMP-9 Treatment

Ability to modify stiffness, plasticity, fragility, adhesivity and thickness of pancreatic islets would allow to solve a number of needs in islet transplantation for treatment of diabetes patients. However, it is highly desirable to maintain functional cohesion between cells within islet, as interaction between cells are important for proper islet function.

Assay:

Islets of Langerhans were isolated according to standard protocols and incubated for 1 days at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed.

Results:

Islets treated by MMP-9 (1) increased adhesivity, (2) increased plasticity and ability to reduce thickness, (3) increased cohesiveness between cells undergoing deformation. Tissue samples treated by collagenase of Clostridium histolyticum were dissociated into non-cohesive floating microscopic particles. Tissue samples treated by buffer (1) retained original stiffness and ability to maintain shape and (2) remained cohesive. MMP-9 treated islets could be deformed to high extent while cells remained cohesive and no ruptures occurred, compared to buffer-treated islets that ruptured under same deformation.

Table 5 shows summary of qualitative assays.

FIG. 23 demonstrates effect of increased plasticity and increased cohesiveness within of MMP-9 treated pancreatic islets, compared to buffer-treated control that underwent rupture under same load.

Eye Lens: Reduced Stiffness and Increased Plasticity after MMP-9 Treatment

A method to reduce stiffness and reduce fragility of eye lens would be desirable in ophthalmology, however, that such method would not rupture the lens and optic nerve.

Assay 1:

Eye lenses were isolated from eyes of same healthy animal for MMP-9 and for buffer treatment to avoid potential differences in stiffness due to age and background. Eye lens for collagenase from Clostridium histoliticum was derived from animal of same age and condition. The eye lens samples were incubated for 4 days at 37° C. in solution of: (1) MMP-9, 0.1 mg/ml, preactivated with 0.5 mM APMA, (2) collagenase of Clostridium histolyticum, 0.1 mg/ml, (3) buffer solution. To assay compatibility with nerve integrity, uniform samples of optic nerve were isolated and were treated by same enzymes formulation at same conditions.

Results:

Tissue samples treated by MMP-9 maintained functional integrity and were less less fragile: when pressure applied, the samples responded with elastic deformation, but not rupture. Tissue samples treated by buffer (1) retained original stiffness and shape and (2) responded to pressure by rupture, but not by elastic deformation.

Tissue samples treated by collagenase of Clostridium histolyticum were dissociated into non-cohesive floating microscopic particles.

Table 5 shows summary of qualitative assays.

FIG. 24 illustrates effect of MMP-9 treated eye lens is less prone to rupture, compared to buffer-treated control.

Nerve: Maintained Integrity and Reduced Stiffness after MMP-9 Treatment

Many medical and veterinary application that involve use of proteolytical enzyme on tissues would benefit, if risk of nerve rupture was reduced (such reducing risks of pain or numbness). Integrity of nerve bundles in important for most enzymatic therapies.

Assay:

Uniform samples of (1) sciatic nerve or (2) optic nerve were isolated by methods of microsurgery and were incubated for up to 4 days at 37° C. in solution of: (1) MMP-9, 0.1 mg/ml, preactivated with 0.5 mM APMA, (2) collagenase of Clostridium histolyticum, 0.1 mg/ml, (3) buffer solution. Samples were gently rinsed by applied solution daily.

The assay described hereby served as a control for several other tissues (see above)

Results:

Tissue samples treated by MMP-9 (1) maintained integrity of single nerves and of whole nerve bundle (no ruptures were observed, (2) integrity on nerves maintained also under deformation, (3) stiffness (stress-strain ratio) of MMP-9 treated sample reduced, thus allowing increased range of cohesive nerve deformation. Tissue samples treated by collagenase of Clostridium histolyticum were dissociated into non-cohesive floating microscopic particles. Tissue samples treated by buffer (1) retained original stiffness and ability to maintain shape and (2) remained cohesive.

Table 5 shows summary of qualitative assays.

### MMP-9 Reduces Stiffness of Muscle.

Tissue samples: (1) muscle samples including muscle fibers wrapped in endomysium and perimysium were subject to qualitative stress-strain assay (deformation under load); (2) muscle samples including myotendonous junction were used for qualitative assessment and to control integrity of myotendonous junction; (3) optic nerve and sciatic nerves were used as a control: being subject to enzyme of same formulation (concentration, activity, buffer composition, etc) in order to control integrity of nerves.

Results:

MMP-9 treatment for 3 days at 37 C in concentration of 0.1 mg/ml allowed mild (though noticeable) reduction of muscle sample and increased plasticity (compared to buffer-treated control). Functional integrity of muscle, myotendonous junction and control nerve sample remained conserved: no rupture within muscle fibers, nerve fibers, endomysium, perimysium, myotendonous junctions observed and muscle fibers remain cohesive to each other within sample; also in applied pressure assay. Tissue samples treated by collagenase of Clostridium histolyticum were digested into non-cohesive floating microscopic particles (1) individual muscle fibers were digested into particles, (2) connection between neighboring fibers was lost, (3) myotendonious junction was digested. Tissue samples treated by buffer (1) retained original stiffness and ability to maintain shape and (2) remained cohesive.

Table 5 shows summary of qualitative assays.

TABLE 1
List of individual enzymes belonging to group of tissue-remodeling enzymes (TREZ):

| # | MMP | Alternative name(s) | EC# | UniProt# |
|---|---|---|---|---|
| 01 | MMP-1 | Interstitial collagenase; Fibroblast collagenase; Matrix | EC = 3.4.24.7 | P03956 (MMP1_HUMAN) |
| | | metalloproteinase-1 | | |
| 02 | MMP-2 | 72 kDa type IV collagenase, 72 kDa gelatinase, Gelatinase A, | EC = 3.4.24.24 | P08253 (MMP2_HUMAN) |
| | | Matrix metalloproteinase-2 | | |
| 03 | MMP-3 | Stromelysin-1, Matrix metalloproteinase-3, Transin-1 | EC = 3.4.24.17 | P08254 (MMP3_HUMAN) |
| 04 | MMP-7 | Matrin, Matrix metalloproteinase-7, Pump-1 protease, Uterine | EC = 3.4.24.23 | P09237 (MMP7_HUMAN) |
| | | metalloproteinase | | |
| 05 | MMP-8 | Neutrophil collagenase, Matrix metalloproteinase-8, PMNL | EC = 3.4.24.34 | P22894 (MMP8_HUMAN) |
| | | collagenase, PMNL-CL | | |
| 06 | MMP-9 | Matrix metalloproteinase-9, 92 kDa gelatinase, 92 kDa type IV | EC = 3.4.24.35 | P14780 (MMP9_HUMAN) |
| | | collagenase, Gelatinase B | | |
| 07 | MMP-10 | Stromelysin-2 (SL-2), Matrix metalloproteinase-10, Transin-2 | EC = 3.4.24.22 | P09238 (MMP10_HUMAN) |
| 08 | MMP-11 | Stromelysin-3 (SL-3), Matrix metalloproteinase-11 | | P24347 (MMP11_HUMAN) |
| 09 | MMP-12 | Macrophage metalloelastase, Matrix metalloproteinase-12 | EC = 3.4.24.65 | P39900 (MMP12_HUMAN) |
| 10 | MMP-13 | Collagenase 3, Matrix metalloproteinase-13 | | P45452 (MMP13_HUMAN) |
| 11 | MMP-14, | Matrix metalloproteinase-14, MMP-X1, Membrane-type matrix | EC = 3.4.24.80 | P50281 (MMP14_HUMAN) |
| | MT1-MMP | metalloproteinase 1v(MT-MMP 1, MTMMP1), Membrane-type-1 | | |
| | | matrix metalloproteinase(MT1-MMP, MT1MMP) | | |
| 12 | MMP-15 | Membrane-type matrix metalloproteinase 2 | | P51511 (MMP15_HUMAN) |
| 13 | MMP-16 | Membrane-type matrix metalloproteinase 3 | | P51512 (MMP16_HUMAN) |
| 14 | MMP-17 | Membrane-type matrix metalloproteinase 4 | | Q9ULZ9 (MMP17_HUMAN) |
| 15 | MMP-18 | Matrix metalloproteinase-18, Collagenase-4 (xCol4) | | O13065 (MMP18_XENLA) |
| | | | | (Xenopus seq.) |
| 16 | MMP-19 | Matrix metalloproteinase RASI, Matrix metalloproteinase-18 | | Q99542 (MMP19_HUMAN) |
| 17 | MMP-20 | Enamel metalloproteinase, Enamelysin | | O60882 (MMP20_HUMAN) |
| 18 | MMP-21 | Matrix metalloproteinase-21 | | Q8N119 (MMP21_HUMAN) |
| 19 | MMP-23A | Femalysin, MIFR-1, MMP-22 | | O75900 (MMP23_HUMAN) |
| 20 | MMP-23B | Femalysin, MIFR-1, MMP-22 | | O75900 (MMP23_HUMAN) |
| 21 | MMP-24 | Membrane-type matrix metalloproteinase 5 | | Q9Y5R2 (MMP24_HUMAN) |
| 22 | MMP-25 | Leukolysin, Membrane-type matrix metalloproteinase 6 | | Q9NPA2 (MMP25_HUMAN) |
| 23 | MMP-26 | Endometase, Matrilysin-2 | | Q9NRE1 (MMP26_HUMAN) |
| 24 | MMP-27 | Matrix metalloproteinase-27 | | Q9H306 (MMP27_HUMAN) |
| 25 | MMP_28 | Epilysin | | Q9H239 (MMP28_HUMAN) |
| 26 | matriptase | | (EC 3.4.21.109) | |
| 27 | leukocyte | Neutrophil elastase, Bone marrow serine protease, Elastase-2, | (EC 3.4.21.37) | P08246 (ELNE_HUMAN) |
| | elastase | Human leukocyte elastase (HLE), Medullasin, PMN elastase | | |
| 28 | kallikrein 14 | | (EC 3.4.21.B45) | Q9P0G3 (KLK14_HUMAN) |
| 29 | cathepsin B | APP secretase (APPS), Cathepsin B1 | (EC 3.4.22.1) | P07858 (CATB_HUMAN) |
| 30 | cathepsin L | | (EC 3.4.22.15) | |
| 31 | cathepsin S | | (EC 3.4.22.27) | |
| 32 | cathepsin K | | (EC 3.4.22.38) | |
| 33 | meprin A | | (EC 3.4.24.18) | |
| 34 | matrilysin | | (EC 3.4.24.23). | |
| 35 | Granzyme A | CTL tryptase, Cytotoxic T-lymphocyte proteinase 1, | EC 3.4.21.78 | P12544 (GRAA_HUMAN) |
| | | Fragmentin-1, Granzyme-1, Hanukkah factor | | |
| 36 | Granzyme B | | EC 3.4.21.79 | |
| 37 | Granzyme H | | | P20718 (GRAH_HUMAN) |

Classification Sources: www.pubmed.gov, http://www.uniprot.org, http://enzyme.expasy.org

TABLE 2
Evolutionary functional differences between aggressive (bacterial or
digestive) collagenases and Tissue-Remodeling Enzymes (TREZ)

| # | criteria | Bact. | Digest. | TREZ |
|---|---|---|---|---|
| 1 | Producing organism aims to apply enzyme on it's own tissues | NO | NO | YES |
| 2 | Producing organism aims to keep permanent mammalian tissues | NO | NO | YES |
| | functional | | | |
| 3 | Producing organism aims to preserve the functional integrity and | NO | NO | YES |
| | architecture of aimed tissue | | | |
| 4 | Producing organism aims to preserve cohesive the aimed tissue | NO | NO | YES |
| 5 | Producing organism aims collagenolysis for healthy purposes for | NO | NO | YES |
| | aimed tissue (like: regeneration, expansion, development) | | | |
| 6 | Producing organism aims to preserve safe and functional the | NO | NO | YES |
| | mammalian tissues neighboring to the aimed tissue | | | |
| 7 | Producing organism aims to preserve safe the nerves (axons) | NO | NO | YES |
| | within and around the aimed tissue | | | |

TABLE 3
Summary of functional integrity of muscle and skin

| | | |
|---|---|---|
| | | Example of disease when |
| # | Tissue unit | integrity is failed |

Functional integrity of muscle and tendon

| | | |
|---|---|---|
| 1 | Nerves | Numbness (sensory nerves), pain |
| | | (sensory nerves), paralysis |
| | | (motor nerves) |
| 2 | Muscle fiber wholeness | Muscle rupture, |
| 3 | Muscle fibers: connection | Congenital muscular dystrophy: |
| | between fibers | mutation of collagen VI or merosin |
| 4 | Tendon: retain connection | Tendon rupture |
| | between muscle and sceleton | |

Functional integrity of skin

| | | |
|---|---|---|
| 1a | Wholeness of epidermis layer | Wound, ulcer, blister, |
| 1b | Wholeness of dermis layer | |
| 2 | Connection between dermis | Epidermolysis bullosa |
| | and epidermis (wholeness of | (mutations of genes: |
| | dermal-epidermal junction) | LAMA3, LAMB3, LAMC3) |
| 3 | Hair integration into skin | Alopecia |

TABLE 4
Mechanical characteristics (such as stiffness, stress-strain ratio, fragility, plasticity, etc.) and shape characteristics (such as: shape,
thickness, positioning angle, area, etc) of mammalian permanent tissues can be modified by use of TREZ: translation of
mechanical and shape characteristics into medical and cosmetical applications.
Diseases caused by pathological stiffness of organs and tissues: can be treated by using TREZ to reduce stiffness (stress-strain ration) back to normal

| | | | | How use of TREZ to modify the mechanoelastic |
|---|---|---|---|---|
| | | Evaluation criteria for disease | Mechanoelastic properties of | characteristics can be used for disease |
| # | Disease | and treatment efficacy | tissue resulting the disease | treatment |
| 1. | Adhesive capsulitis | Range of shoulder motility is | Increased stiffness of | TREZ treatment allows to decrease stress-strain |
| | (frozen shoulder), | limited. Degree of pathology is | connective tissues within | ratio, therefore range of strain (range of shoulder |
| | | evaluated as: (1) rotational | shoulder, therefore,ROM is | movements) can be increased within |
| | | range of motions (ROMs) and | decreased in disease. (Lin, | physiological limits of stress (force that does not |
| | | (2) glenohumeral (GH) stiffness | Hsu et al. 2008; Koh, Chung et | cause pain or damage). See FIG. 4. |
| | | | al. 2012) | |
| 2. | Dupuytren's contracture | Angle of finger movement; is | Increased stiffness of | TREZ treatment allows to decrease stress-strain |
| | | limited by stiffened/shortened | Dupuytren's cords/shortened | characteristics of connective tissues; therefore, |
| | | Dupuytren's cords. The range of | shape of Dupuytren's cords. | Dupuytren's cords can be either made more |
| | | finger bending angle is | | elastic, or be extended (shape modification). |
| | | evaluation criteria (Hurst, | | |
| | | Badalamente et al. 2009) | | |
| 3. | Phimosis | Inability of penis foreskin to | Increased stiffness of penis | TREZ treatment allows to reduce stiffness of |
| | | extend enough to allow erection. | foreskin | penis tissues, therefore, healthy range of |
| | | | | foreskin stretching can be restored. See FIG. |
| | | | | 5. |
| 4 | Stiffened eye lens; poor | Accomodation range (range of | Increased stiffness of eye lens | TREZ treatment allows to reduce stiffness |
| | accomodation | distance to which an eye can | | (reduce stress-strain ration), therefore, the range |
| | | focus) | | of strain would increase, therefore, range of eye |
| | | | | accommodation would increase |
| 5 | Stiffened eye lens; | Stiffened eye reacts to external | Increased brittleness, | TREZ treatment allows to make eye lens more |
| | increased fragility | force not by elastic deformation, | increased stiffness (Hansen, | elastic and therefore, less fragile and less prone |
| | | but by rupture (breakage) | Stitzel et al. 2003; Stitzel, | to rupture caused by external force |
| | | | Hansen et al. 2005) | |
| 6 | Liver fibrosis and liver | Stress-strain characteristics | Stiffness of liver is not just a | TREZ can be used to reduce stiffness (reduce |
| | cirrhosis | correlate with extent of liver | consequence, but also part of | stress-strain ration) for liver tissues, therefore, |
| | | fibrosis and cirrhosis. Increased | pathogenesis (Georges, Hui et | allow regenerative processes and restore |
| | | stiffness of liver hinders ability of | al. 2007). | function |
| | | liver to regenerate. | | |
| 7 | Pulmonary fibrosis | Ability of lung for elastic | Increased stiffness of | TREZ can be used to reduce stress-strain ration, |
| | | deformation within healthy | extracellular matrix within | therefore, increase range of lung inflation |
| | | range | lungs | |

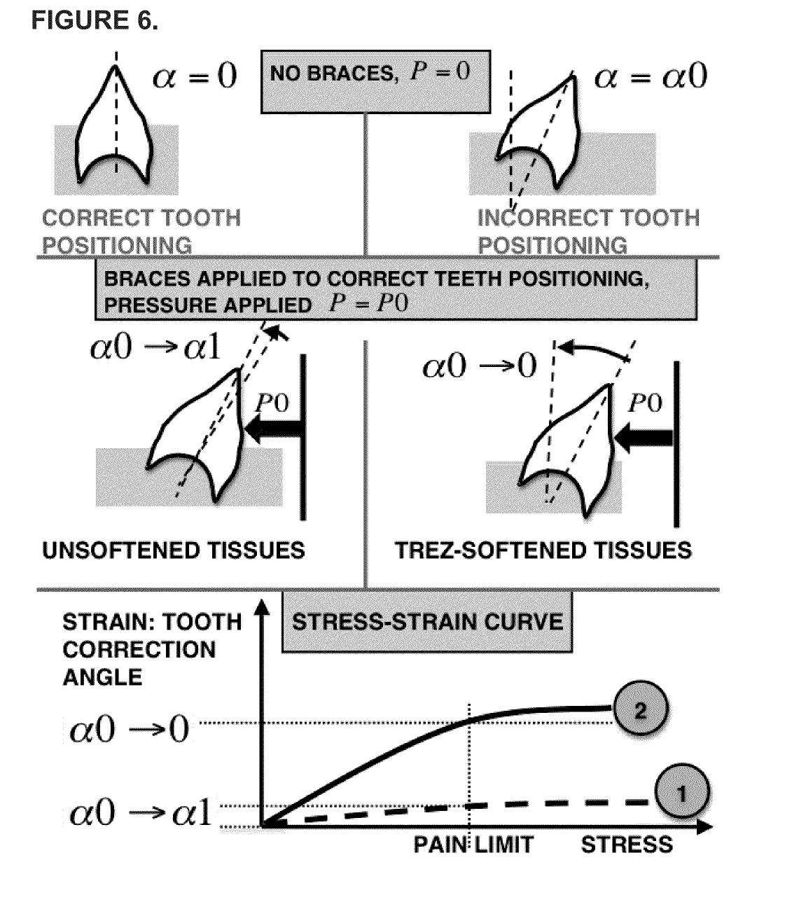

Medical and cosmetical needs for increased plasticity: can be treated by using TREZ to reduce stiffnes, increase plasticity and adhesivity

| | | | | How use of TREZ to modify the mechanoelastic |
|---|---|---|---|---|
| | | Evaluation criteria for disease | Mechanoelastic properties of | characteristics can be used for disease |
| # | Medical procedure | and treatment efficacy | tissue resulting the disease | treatment |
| 8 | Plastic surgery | Shape or organ (nose, ear, | Natural stiffness (stress-strain | TREZ-treatment may be used to make tissues |
| | (rhinoplasty, otoplasty, | breasts, etc) that needs to be | characteristics) of tissues | more plastic, therefore, more easy to attain |
| | mammoplasty, etc) | modified | make tissues “remember” | desired shape. Use of temporary scaffold for |
| | | | original shape. | intended shape would be beneficial (such as: |
| | | | | dental braces for teeth or gold threads for |
| | | | | facelift). |
| 9 | Reconstructive surgery | Extent of cleft (shape, depth of | Stiffness and low adhesivity of | TREZ-treated lip tissues could (1) expand, (2) |
| | for cleft lip. | cleft) | untreated lip tissues | merge to cover the cleft |
| 10 | Incorrect positioning of | Angle of incorrect tooth | Connective tissues around | Applying TREZ to connective tissues around |
| | teeth | positioning | tooth are too stiff to allow easy | tooth may allow to achieve better effect using |
| | | | change of tooth positioning | dental braces. See FIG. 6. |
| 11 | Islet transplantation: | Size of islet | Untreated islets are too stiff to | TREZ treatment may make make islets more |
| | increase survival rate | | allow cohesive deformation | plastic and allow cohesive reshaping into flat |
| | | | into flat shape | structures |
| 12 | Transplantation: | Gaps between transplanted | Natural stiffness and low | TREZ treatment increases plasticity and |
| | adhesion of transplanted | tissue and host tissue | adhesiveness of tissues may | adhesiveness of tissues. If transplant or host |
| | tissue to host tissue | | cause gaps between | tissues are TREZ-treated, plasticity and |
| | | | transplant and host tissues | adhesiveness would allow to reduce gaps. |
| | | | | See FIG. 12. |
| 13 | Scarring in open surgery | Extent of scarring | Natural stiffness and low | TREZ treatment increases plasticity and |
| | | | adhesiveness of tissues may | adhesiveness of tissues: could be used to allow |
| | | | cause gaps between lesion | gapless fusion of lesion borders, thus, reducing |
| | | | borders | risk of scarring. |
| 14 | Wrinkles reduction in | Depth and other geometrical | Natural stiffness of skin dermis | Making skin dermis more plastic, shape of skin |
| | cosmetics | proportions of wrinkles | makes it “remember” original | can be modified to reduce wrinkles. Use of |
| | | | shape, i.e. wrinkles | durable scaffold (for example: gold threads) may |
| | | | | be beneficial. |

TABLE 5
MMP-9 ability to modify (1) stiffness, (2) plasticity, (3) adhesivity, (4) ability to reshape,
(5) fragility in tissues compared to buffer-treated samples

| | | Functional | | | | | |
|---|---|---|---|---|---|---|---|
| | | integrity | Stiffness | Plasticity | Adhesivity | Reshaping | Fragility |
| 1 | Lip | maintains | Decreased, | Increased, | Increased, | Increased, | Not measured |
| | | | highly | highly | highly | highly | |
| 2 | Skin, dermis | maintains | Decreased | Increased | Increased | Increased | Not measured |
| 3 | Tendon (Achilles, | maintains | Decreased | Increased, | Increased, | Increased | Not measured |
| | limb large, finger) | | | highly | highly | permeability | |
| 4 | Cartilage | maintains | Decreased | Increased | Not measured | Increased | Not measured |
| | (ear, nose) | | | | | | |
| 5 | Penis foreskin | maintains | Decreased, | Increased, | Increased | Increased | Not measured |
| | | | Highly | highly | | | |
| 6 | Penis, connective | maintains | Decreased | Increased | Increased | Increased | Not measured |
| | tissues | | | | | | |
| 7 | Liver | maintains | Decreased | Increased | Not measured | Increased | Decreased, cells |
| | | | | | | | more cohesive |
| 8 | Uterus | maintains | Decreased | Increased | Increased | Increased | Not measured |
| 9 | Ovaries | maintains | Decreased | Increased | Not measured | Increased | Not measured |
| 10 | Pancreas | maintains | Not measured | Not measured | Not measured | Increased | Not identified |
| | | | | | | permeability | |
| 11 | Pancreatic islets | maintains | Decreased | Not measured | Increased | Not measured | Decreased, cells |
| | | | | | | | remain cohesive |
| 12 | Eye lens | maintains | Not measured | Not identified | Not identified | Not identified | Decreased |
| 13 | Nerve | maintains | Decreased | Increased | Not measured | Not measured | Not measured |
| 14 | Muscle | maintains | Decreased, | Not measured | Not measured | Not measured | Not measured |
| | | | moderately | | | | |

### REFERENCES

### Articles

Carroll, E. P., J. S. Janicki, et al. (1989). “Myocardial stiffness and reparative fibrosis following coronary embolisation in the rat.” Cardiovasc Res 23(8): 655-661.
Dechene, A., J. P. Sowa, et al. (2010). “Acute liver failure is associated with elevated liver stiffness and hepatic stellate cell activation.” Hepatology 52(3): 1008-1016.
Discher, D. E., P. Janmey, et al. (2005). “Tissue cells feel and respond to the stiffness of their substrate.” Science 310(5751): 1139-1143.
Engler, A. J., S. Sen, et al. (2006). “Matrix elasticity directs stem cell lineage specification.” Cell 126(4): 677-689.
Engler, A. J., H. L. Sweeney, et al. (2007). “Extracellular matrix elasticity directs stem cell differentiation.” J Musculoskelet Neuronal Interact 7(4): 335.
Friedman, S. L. (2010). “Evolving challenges in hepatic fibrosis.” Nat Rev Gastroenterol Hepatol 7(8): 425-436.
Georges, P. C., J. J. Hui, et al. (2007). “Increased stiffness of the rat liver precedes matrix deposition: implications for fibrosis.” Am J Physiol Gastrointest Liver Physiol 293(6): G1147-1154.
Hansen, G. A., J. D. Stitzel, et al. (2003). “Incidence of elderly eye injuries in automobile crashes: the effects of lens stiffness as a function of age.” Annu Proc Assoc Adv Automot Med 47: 147-163.
Hirayasu, H., Y. Yoshikawa, et al. (2008). “A lymphocyte serine protease granzyme A causes detachment of a small-intestinal epithelial cell line (IEC-6).” Biosci Biotechnol Biochem 72(9): 2294-2302.
Hurst, L. C., M. A. Badalamente, et al. (2009). “Injectable collagenase clostridium histolyticum for Dupuytren's contracture.” N Engl J Med 361(10): 968-979.
Klasen, H. J. (2000). “A review on the nonoperative removal of necrotic tissue from burn wounds.” Burns 26(3): 207-222.
Koh, E. S., S. G. Chung, et al. (2012). “Changes in biomechanical properties of glenohumeral joint capsules with adhesive capsulitis by repeated capsule-preserving hydraulic distensions with saline solution and corticosteroid.” PM R 4(12): 976-984.
Lehmann, R., R. A. Zuellig, et al. (2007). “Superiority of small islets in human islet transplantation.” Diabetes 56(3): 594-603.
Li, D. S., Y. H. Yuan, et al. (2009). “A protocol for islet isolation from mouse pancreas.” Nat Protoc 4(11): 1649-1652.
Lin, H. T., A. T. Hsu, et al. (2008). “Reliability of stiffness measured in glenohumeral joint and its application to assess the effect of end-range mobilization in subjects with adhesive capsulitis.” Man Ther 13(4): 307-316.
Starkweather, K. D., S. Lattuga, et al. (1996). “Collagenase in the treatment of Dupuytren's disease: an in vitro study.” J Hand Surg Am 21(3): 490-495.
Stitzel, J. D., G. A. Hansen, et al. (2005). “Blunt trauma of the aging eye: injury mechanisms and increasing lens stiffness.” Arch Ophthalmol 123(6): 789-794.
Wang, J. H., C. S. Changchien, et al. (2009). “FibroScan and ultrasonography in the prediction of hepatic fibrosis in patients with chronic viral hepatitis.” J Gastroenterol 44(5): 439-446.
Wang, M. H., M. L. Palmeri, et al. (2009). “In vivo quantification of liver stiffness in a rat model of hepatic fibrosis with acoustic radiation force.” Ultrasound Med Biol 35(10): 1709-1721.
Wells, R. G. (2005). “The role of matrix stiffness in hepatic stellate cell activation and liver fibrosis.” J Clin Gastroenterol 39(4 Suppl 2): S158-161
“Matrix Metalloproteinase Protocols” by Clark, Ian M, published 2001, Methods in Molecular Biology, ISBN 1592590462, Vol. 151

### Patents

“Wound healing composition”, U.S. Pat. No. 3,003,917 (A), 1961 Oct. 10 by Beiler et. al.
U.S. Pat. No. 6,365,405, by Salzmann et. al. (2002) “Compositions of chondrocytes, preparation and utilization”
U.S. Pat. No. 8,323,642 by Story et al. (2012) “Tissue fusion method using collagenase for repair of soft tissue”
US patent application 20080145357 by Story et. al. (2008) “Tissue fusion method using collagenase for repair of soft tissue”
US patent application 20100247512, by Weiss et. al. (2010) “Pericellular Collagenase Directs the 3-Dimensional Development of White Adipose Tissue”
U.S. Pat. No. 5,922,322, by Bini et. al. (1999) “Fibrin(ogen) degradation and clot lysis by fibrinolytic matrix metalloproteinase”
US patent application 20110091436, by Orbe et. al. (2011) “USE OF MATRIX METALLOPROTEINASE-10 (MMP-10) FOR THROMBOLYTIC TREATMENTS”
“Composition for revitalizing scar tissue”, EP0637450 (A2); EP0637450 (A3), 1995 Feb. 8, by Berg et. al.
“IN VIVO TEMPORAL CONTROL OF ACTIV AT ABLE MATRIX-DEGRADING ENZYMES”, WO2009111083 (A2); WO2009111083 (A3), 2009 Sep. 11, by Keller et. al.
U.S. Pat. No. 7,935,337 by Haro (2011) “Method of therapy for degenerative intervertebral discs”
US patent application 20110044960 by Nakashima et. al. (2011) “MEDICAMENT, DENTAL MATERIAL, AND METHOD OF SCREENING”

1-16. (canceled)

17. A method of treating a condition which requires a modification of mechanical properties of permanent mammalian tissue while the tissue remains cohesive, comprising applying a human MMP to said tissue.

18. The method according to claim 17, wherein the human MMP is a gelatinase.

19. The method according to claim 18, where the gelatinase is MMP-9.

20. The method according to claim 17, wherein the condition is an elevated stiffness-associated condition selected from the group consisting of liver fibrosis, lung fibrosis, heart fibrosis and uterine fibrosis.

21. The method according to claim 17, wherein the tissue is selected from the group consisting of connective, epithelial, neural or muscular tissue in cartilage; bone; muscle; tendon; skin; liver; pancreas; pancreatic islets of Langerhans, uterus; ligament; fascia; eye lens; kidney; ovaries; lung; intestine; esophagus; stomach; duodenum; gall bladder; thymus glands; salivary glands; mammary glands; penis; vagina; labia; facial tissues and lip.

22. The method according to claim 17, wherein the treated tissue retains cohesion when tissue is subject to static or dynamic strain or deformation.

23. The method according to claim 17, wherein said mechanical properties is one or more properties selected from the group consisting of stiffness, plasticity, elasticity, fragility, brittleness, mechanoelasticity, viscoelasticity, adhesiveness, ability to reduce risk of ruptures, ability to reduce risk of erosion and any stress-strain characteristics.

24. The method according to claim 23, wherein the modification of the mechanical properties is a decrease in stiffness (stress/strain ratio) of the tissue, preferably to over 1%, more preferably to over 10% and most preferably to over 30% in treated tissue as compared to non-treated.

25. The method according claim 17, wherein the modification of the mechanical properties of the tissue is an increase of the limits of cohesive deformation (strain) or range of motion (ROM) of the tissue, preferably to over 1%, more preferably to over 10% and most preferably to over 30% in treated tissue as compared to non-treated.

26. The method according to claim 23, wherein the modification of the mechanical properties is an increase in the plasticity of the tissue, preferably to over 1%, more preferably to over 10% and most preferably to over 30% in treated tissue as compared to non-treated.

27. The method according to claim 17, wherein the modification of the mechanical properties of the tissue is the shape parameters of the tissue, such as surface area, thickness, curvature, shape, characteristic angles or positioning, which are modified by the treatment to over 1%, preferably to over 10% and more preferably to over 30% in treated tissue as compared to non-treated.

28. The method according to claim 24, wherein the medical condition involves increased stiffness of said tissues, the human MMP is for use in relation to fibrosis, plastic surgery and/or ophthalmology, and possible applications are any of: liver fibrosis, liver cirrhosis, pulmonary fibrosis, myocardial fibrosis, liver fibrosis, myelofibrosis, fibrosis of uterus, Crohn's Disease, permanent tissue fibrosis, Dupuytren's contracture, phimosis, adhesive capsulitis (stiff shoulder), and stiffened eye lens.

29. The method according to claim 24, wherein the medical condition involves reduced range of deformation (strain) of ligaments or connective tissues and the medicament is for use in relation to adhesive capsulitis, Dupuytren's contracture, phimosis, abnormally stiffened eye lens.

30. The method according to claim 24, wherein the treating of a condition involves correction of skin shape and stress-strain parameters, such as: wrinkles, lesions, elevated stiffness, insufficient ability to stretch.

31. The method according to claim 24, wherein the treating of a condition involves correction of tooth positioning, for example, in combination with dental braces.

32. The method according to claim 24, wherein the treating of a condition involves correction on tissues shape and possible applications are selected from the group consisting of: plastic surgery and cosmetics, such as wrinkle correction, treatment of Peyronie's disease, fillers injections, abdominoplasty, blepharoplasty, phalloplasty, mammoplasty, buttock reshaping, labiaplasty, vagina reconstruction, lip plastic surgery, cleft lip correction, rhinoplasty, otoplasty, rhytidectomy, browplasty, midface lift, reshaping of facial bones and soft tissues, orthognathic surgery, brachioplasty, removal of scars, removal of warts, and removal of papillomas.

33. The method according to claim 24, wherein the treating of a condition is for surgical use such as transplantation, grafting, flap-transplantation, expanding transplant tissues, tissue fusion, reconstructive surgery, debridement, reducing the risk of scarring, reducing the risk of rupture, reducing erosion, treatment of ruptures, treatment of erosions, treatment of lesions, orthopedic applications, tumor removal, and/or for implants insertion.

34. The method according to claim 24, wherein the treating of a condition is for reproductive medicine use, such as to improve fertility, to reduce risk of spontaneous abortion, to reduce risk of fetus ischemia, to reduce risks of invasive prenatal diagnostics methods, for cervix softening, for treatment of post-delivery ruptures.

35. The method according to claim 24, wherein method is intended for regenerative medicine use.

36. A preparation for modification of the mechanical properties, such as shape, of permanent human tissue for transplantation, which preparation comprises isolated MMP-9.

37. The preparation according to claim 36 for the treatment of isolated pancreatic islets of Langerhans ex vivo.

38. The preparation according to claim 37 capable of modifying the shape of islets.

39. The preparation according to claim 36 used for treatment of connective tissues or ligaments.

40. The preparation according to claim 39, wherein connective tissue is a cartilage graft.

41. The preparation according to claim 39, wherein the connective tissue is a skin graft.

42. The preparation according to claim 39, wherein the connective tissue is a lip graft.

43. A preparation comprising isolated MMP-9 for subdermal injection and treatment of a condition selected from the group consisting of Dupuitren's contracture, Peyronie's disease, adhesive capsulitis, and phimosis.

44. A preparation for the treatment of fibrosis, which preparation comprises isolated MMP-9.

45. The preparation according to claim 44, wherein the fibrosis is liver fibrosis.

46. A preparation for skin care and cosmetics, which preparation comprises isolated MMP-9.

47. A method of treating a mammalian body, which comprises application of human MMP to mammalian tissue, preferably permanent tissue, excluding scar, blood clot and fat, to provide a modification of mechanical properties, such as stiffness, plasticity and elasticity or range of motion (ROM) or shape of said tissue, while the tissue remains cohesive.

48. The method of treatment according to claim 47, wherein the range of cohesive strain is increased in treated tissues.

49. The method of treatment according to claim 47, wherein the MMP is gelatinase.

50. The method of treatment according to claim 49, wherein the gelatinase is MMP-9.

51. The method of treatment according to claim 47 which is a method for surgery such as transplantation, grafting, flap-transplantation, expanding transplant tissues, tissue fusion, reconstructive surgery, debridement, reducing the risk of scarring, reducing the risk of rupture, reducing erosion, treatment of ruptures, treatment of erosions, treatment of lesions, orthopedic applications, tumor removal, and/or for implants insertion.

52. The method according to claim 47 which is for plastic surgery and cosmetics, such as wrinkle correction, treatment of Peyronie's disease, fillers injections, abdominoplasty, blepharoplasty, phalloplasty, mammoplasty, buttock reshaping, labiaplasty, vagina reconstruction, lip plastic surgery, cleft lip correction, rhinoplasty, otoplasty, rhytidectomy, browplasty, midface lift, reshaping of facial bones and soft tissues, orthognathic surgery, brachioplasty, removal of scars, removal of warts, and removal of papillomas.

53. The method of treatment according to claim 47, which is a method of reduction of stiffness in tissues with increased stiffness.

54. The method according to claim 53, which is a method of treatment of stiffened tissues, such as liver fibrosis, liver cirrhosis, pulmonary fibrosis, myocardial fibrosis, fibrosis of uterus, Crohn's Disease.

55. The method according to claim 47, wherein the range of cohesive strain (deformation) or range of motion (ROM) of the tissue is increased by the treatment, preferably to over 1%, more preferably to over 10% and most preferably to over 30% in treated tissue as compared to non-treated.

56. The method according to claim 55, wherein the method is for treatment associated with decreased range of motion (ROM), such as Dupuytren's contracture, adhesive capsulitis, and/or stiffened eye lens.

57. The method according to claim 47 which improves the range of vision in said subject by restoring eye lens range of cohesive strain (deformation) or range of motion (ROM).

58. The method according to claim 47, which is for the modification of shape of tissue in the mammalian body.

59. The method according to claim 58, wherein a tissue shape parameter selected from the group consisting of a reduction of tissue unit thickness, an increase in tissue unit surface area, a modification of tissue shape or positioning, and range of cohesive strain (deformation), is increased by 1% or more as compared to untreated tissue, preferably by 30% or more.

60. The method according to claim 58, which comprises the steps of treatment by a mammalian collagenase; and applying molding deformation to shape tissue into a desired shape.

61. The method of treatment according to claim 47, which is reproductive medicine, such as to improve fertility, to reduce risk of spontaneous abortion, to reduce risk of fetus ischemia, to reduce risks of invasive prenatal diagnostics methods, for cervix softening, for treatment of post-delivery ruptures.

62. The method of treatment according to claim 47, which is for use in regenerative medicine.

63. A method of contacting an isolated MMP with mammalian tissue, preferably permanent tissue, excluding scar, blood clot and fat, to provide a modification of mechanical properties, range of motion (ROM) or shape of said tissue, wherein said method is for in vitro use, tissue cultivation, isolation and processing of pancreatic islets, changing shape of graft material for transplantation, processing of decellularized tissues, food industry, embalming, fur industry, and/or hyde industry, said MMP is applied to the tissue, and the tissue remains cohesive.

64. The method according to claim 63, wherein the MMP is gelatinase.

65. The method according to claim 64, wherein the gelatinase is MMP-9.

66. The method according to claim 64, wherein said tissue is pancreatic islets of Langerhans.

67. The method according to claim 66, wherein treatment of said islets with said composition is followed by strain that would reduce islet thickness.

68. The method according to claim 63, wherein said tissue is graft for transplantation such as skin, cartilage, tendon, and/or any ligaments or connective tissues.
